(19) United States
(12) Patent Application Publication
(10) Pub. No.: US 2022/0227762 A1
(43) Pub. Date: Jul. 21, 2022

(54) AMIDE-SUBSTITUTED IMIDAZO COMPOUNDS AS SELECTIVE INHIBITORS OF INDOLEAMINE 2,3-DIOXYGENASES

(71) Applicant: BEIGENE, LTD., Camana Bay, Grand Cayman (KY)

(72) Inventors: Hexiang WANG, Beijing (CN); Ming QIU, Beijing (CN); Changyou ZHOU, Beijing (CN)

(21) Appl. No.: 17/612,056

(22) PCT Filed: May 21, 2020
(86) PCT No.: PCT/CN2020/091602

§ 371 Date: Nov. 17, 2021

(30) Foreign Application Priority Data
May 22, 2019 (CN) PCT/CN2019/087983

(51) Int. Cl. C07D 471/04; C07D 401/08; C07D 405/14

(52) U.S. Cl. CPC C07D 471/04; C07D 405/14; C07D 401/08

(57) ABSTRACT

Disclosed herein are amide-substituted imidazo compounds and pharmaceutical compositions comprising at least one such novel benzoimidazoles, processes for the preparation thereof, and the method for using the same in therapy. In particular, disclosed herein are certain amide-substituted imidazo compounds that are useful for inhibiting indoleamine 2, 3-dioxygenase and for treating diseases or disorders mediated thereby.

### FIELD OF THE INVENTION

Disclosed herein are amide-substituted imidazo compounds and pharmaceutical compositions comprising at least one such compound, processes for the preparation thereof, and the method for using the same in therapy. In particular, disclosed herein are certain amide-substituted imidazo compounds that are useful for inhibiting indoleamine 2,3-dioxygenase and for treating diseases or disorders mediated thereby.

### BACKGROUND OF THE INVENTION

Indoleamine 2,3-dioxygenase 1 (IDO1, EC 1.13.11.42, also known as indoleamine 2,3-dioxygenase) is the first and rate-limiting enzyme in the tryptophan-kynurenine pathway that degrades the essential amino acid L-tryptophan (L-Trp) to N-formal-kynurenine, which can be subsequently metabolized through a series of steps to form NAD. IDO1 enzyme is expressed in the placenta, the mucosal and lymphoid tissues, and in inflammatory lesions (Yamazaki F, et. al., Biochem J. 1985; 230:635-8; Blaschitz A, et, al., PLoS ONE. 2011; 6:e21774). In the latter two, it is expressed primarily by antigen-presenting cells (APC), mainly dendritic cells (DC) and macrophages, and in cells exposed to interferon-gamma (IFNγ) and other pro-inflammatory stimuli. In human cells, the depletion of L-Trp resulting from IDO1 activity as well as the production of a series of immunoregulatory metabolites, collectively known as “kynurenines”, can suppress the proliferation and differentiation of effector T cells [Frumento G, et. al., (2002). Journal of Experimental Medicine 196: 459-468], and markedly enhance the suppressor activity of regulatory T cells [Sharma M D, et al. (2009), Blood 113: 6102-6111], As a result, IDO1 controls and fine-tunes both innate and adaptive immune responses [Grohmann U, et al. (2002), Nature Immunology 3: 1097-1101] under a variety of conditions, including pregnancy [Munn et al. (1998), Science 281: 1191-1193], transplantation [Palafox D, et al. (2010), Transplantation Reviews 24: 160-165], infection [Boasso A, et al. (2009), Amino Acids 37: 89-89], chronic inflammation [Romani L, et al. (2008), Nature 451: 211-U212], autoimmunity [Platten M, et al. (2005), Science 310: 850-855], neoplasia, and depression [Macs M, et, al., Life Sci. 2002 6; 71(16): 1837-48; Myint A M, et. al., (2012), Journal of Neural Transmission 119: 245-251].

Several lines of evidence suggest that IDO is involved in induction of immune tolerance. The immunosuppressive effect of IDO1 was demonstrated first in a mouse model of fetal protection against maternal immune rejection. Treatment of pregnant mice with a tryptophan analog that inhibits IDO1, which is constitutively expressed in the placenta, resulted in T cell-mediated rejection of allogeneic embryos [Munn D H, et al. (1998), Science 281: 1191-1193]. Subsequent studies developed this concept as a mechanism to defeat immune surveillance in cancer (reviewed in [Prendergast G C (2008), Oncogene 27(28):3889-3900; Munn D H, et. al., (2007), J Clin Invest 117(5):1147-1154]. Indoleamine 2,3-dioxygenase is widely overexpressed in tumor cells where it has been associated predominantly with poor prognosis [Uyttenhove C, et. al., (2003), Nat Med 9(10):1269-1274; Liu X, et. al., (2009), Curr Cancer Drug Targets 9(8):938-95], Expression of IDO by immunogenic mouse tumor cells prevents their rejection by preimmunized mice [Uyttenhove C. et. al., Nat Med. 2003 October; 9(10):1269-74. Epub 2003 Sep. 21]. IDO activity is shown to suppress T cells [Fallarino F, et. al., (2002), Cell Death Differ 9:1069-1077; Frumento G, et. A, (2002), J Exp Med 196(4):459-468; Terness P. et. al., (2002), J Exp Med 196(4):447-457] and NK cells [Della Chiesa M, et, al., (2006). Blood 108(13):4118-4125], and also that IDO was critical to support the formation and activity of Tregs [Fallarino F, et al., (2003), Nat Immunol 4(12):1206-1212] and myeloid-derived suppressor cells (NIDSCs) [Smith C, et. al., (2012), Cancer Discovery 2(8):722-735]. It has been suggested that the efficacy of therapeutic vaccination of cancer patients might be improved by concomitant administration of an IDO inhibitor [Uyttenhove C. et. al., Nat Med. 2003 October; 9(10):1269-74. Epub 2003 Sep. 21], It has been shown that the IDO inhibitor, 1-MT, can synergize with chemotherapeutic agents to reduce tumor growth in mice, suggesting that IDO inhibition may also enhance the anti-tumor activity of conventional cytotoxic therapies [Muller A J, et. al., Nat Med. 2005 March; 11(3):312-9]. It has also been shown that IDO inhibitors can synergize with anti-CTLA-4 antibody or anti-PD L-1 antibody in inhibiting tumor growth in mouse models [Holmgaard R B, et. al., J Exp Med. 2013 Jul. 1; 210(7):1389-402; Spranger S, et. al., J Immunother Cancer. 2014, 2:3].

It has been proposed that IDO is induced chronically by HIV infection, and is further increased by opportunistic infections, and that the chronic loss of Trp initiates mechanisms responsible for cachexia, dementia and diarrhea and possibly immunosuppression of AIDS patients [Brown, et al., 1991, Adv. Exp, Med. Biol., 294: 425-35]. To this end, it has recently been shown that IDO inhibition can enhance the levels of virus-specific T cells and, concomitantly, reduce the number of virally infected macrophages in a mouse model of HIV [Portula et al., 2005, Blood, 106:2382-90]. Simian Immunodeficiency Virus (SW) is very similar to Human Immunodeficiency Virus (HIV) and it is used to study the condition in animal models. In both HIV and SIV, the level of virus in the blood, or ‘viral load’, is important because when the viral load is high, the disease progress and it depletes the patient's immune system. This eventually leads to the onset of Acquired Immune Deficiency Syndrome (AIDS), where the patient cannot fight infections which would be innocuous in healthy individuals. It has also been reported that monkeys with the simian form of HIV treated with an IDO inhibitor, called D-1mT alongside Anti-Retroviral Therapy (ART), reduced their virus levels in the blood to undetectable levels, therefore when combined with ARTs, IDO inhibitors may help HIV patients not responding to treatment in the future [Adriano Boasso, et. al., J. Immunol., April 2009; 182: 4313-4320].

In light of the experimental data indicating a role for IDO in immunosuppression, tumor resistance and/or rejection, chronic infections, HIV-infection, AIDS (including its manifestations such as cachexia, dementia and diarrhea), autoimmune diseases or disorders (such as rheumatoid arthritis) and depression, therapeutic agents aimed at suppression of tryptophan degradation by inhibiting IDO activity are of interests. Inhibitors of IDO can be used as effective cancer therapy as they could reverse the immunosuppressive effects of tumor microenvironment and activate anti-tumor activity of T cells, IDO inhibitors could also be useful in activation of immune responses in HIV infection, Inhibition of IDO may also be an important treatment strategy for patients with neurological or neuropsychiatric diseases or disorders such as depression. The compounds, compositions and methods herein help meet the current need for IDO modulators.

Tryptophan 2,3-dioxygenase (TDO, EC 1.13.11.11) catalyzes the same Trp degradation reaction as IDO1, TDO is primarily expressed in the liver in humans, where acts as the main regulator of systemic tryptophan levels. More recently, TDO was also found to be expressed in the brain, where it may regulate the production of neuroactive tryptophan metabolites such as kynurenic acid and quinolinic acid [Kanai M, et. al., Mol. Brain 2009; 2:8]. Two recent studies [Opitz C A, et. al., Nature 2011; 478:197-203; Pilotte L, et. al., Proc Natl Acad Sci USA. 2012, 109(7):2497-502] point to the significance of TDO activity in certain cancers where it is expressed constitutively (particularly malignant glioma, hepatocellular carcinoma, melanoma, and bladder cancer). Functional studies in human tumors indicate that constitutive TDO enzymatic activity is sufficient to sustain biologically relevant tryptophan catabolism that is capable of suppressing antitumor immune responses [Opitz C A, et. al., Nature 197-Pilotte L, et. al., Proc Natl Acad Sci USA. 2012, 109(7):2497-502]. TDO expression by tumors is reported to prevent rejection by immunized mice. A specific TDO inhibitor is shown to restore the ability of mice to reject TDO-expressing tumors without causing significant toxicity [Pilotte L, et. al., Proc Natl Acad Sci USA. 2012, 109(7):2497-502]. Therefore, inhibitors of TDO can potentially be used as a single agent or in combination with other anti-cancer therapies to treat a variety of human cancers.

Small molecule inhibitors of IDO are being developed to treat or prevent IDO-related diseases such as those described above. For example, PCT Publication WO 99/29310 reports methods for altering T cell-mediated immunity comprising altering local extracellular concentrations of tryptophan and tryptophan metabolites, using an inhibitor of IDO such as 1-methyl-DL-tryptophan, p-(3-benzofuranyl)-DL-alanine, p-[3-benzo(b)thienyl]-DL-alanine, and 6-nitro-L-tryptophan) (Munn, 1999). Reported in WO 03/087347, also published as European Patent 1501918, are methods of making antigen-presenting cells for enhancing or reducing T cell tolerance (Munn, 2003). Compounds having indoleamine-2,3-dioxygenase (IDO) inhibitory activity are further reported in WO 2004/094409; WO 2006/122150; WO 2009/073620; WO 2009/132238; WO 2011/056652, WO 2012/142237; WO 2013/107164; WO 2014/066834; WO 2014/081689; WO 2014/141110; WO 2014/150646; WO 2014/150677; WO 2015006520; WO 2015/067782; WO 2015/070007; WO 2015/082499; WO 2015/119944; WO 2015/121812; WO 2015/140717; WO 2015/150697; WO 2015/173764; WO2015/188085; WO 2016/026772; WO 2016/024233; WO2016/026772; WO 2016/037026; WO 2016/040458; WO 2016/051181; WO 2016/059412; WO 2016/071283; WO 2016/071293; WO 2016/073738; WO 2016/073770; WO 2016/073774; US 2015328228; US 2015266857; WO 2016/155545; WO 2016/161279; WO 2016/161279; WO 2016/161269; WO 2016/165613; WO 2016/16942; 1 WO 2016/210414; WO 2017/002078; WO 2017/007700; WO 2017/024996; WO 2017/075341; WO 2017/101884; WO 2017/106062; WO 2017/117393; WO 2017/120591; WO 2017/124822; WO 2017/129139; WO 2017/133258; WO 2017/134555; WO 2017/139414; WO 2017/140272; WO 2017/140274; WO 2017/143874; WO 2017/149469; WO 2017/152857; WO 2017/153459; WO 2017/181849; WO 2017/185959; WO 2017/189386; WO 2017/192811; WO 2017/192815; WO 2017/192813; WO 2017/192840; WO 2017/192844; WO 2017/19514; WO 2018/039512, In particular, WO 2018/039512 discloses benzo[d]imidazole, imidazo[1,2-b]pyridazine, imidazo[1,2-b]pyridine and some other compounds as showing IDO inhibitory activity.

However, there is an unmet need of IDO inhibitors exhibiting potent inhibitory activity for IDO as well as showing lower human plasma protein binding affinity, which may reach to the maximum efficacy using lower drug to higher free drug fraction in human body.

### SUMMARY OF THE INVENTION

Unexpectedly, the inventors of the present invention found that substitution of an amide group at the imidazo compounds imparted potent IDO1 inhibitory activity and significantly lowered human plasma protein binding affinity of the amide-substituted imidazo compounds compared with the non-amide compounds.

Disclosed herein is a compound of Formula (I)

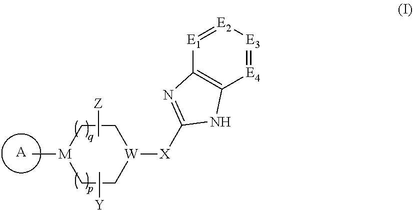

or a stereoisomer thereof, or a pharmaceutically acceptable salt thereof,

wherein:

M is CH or N;

W is CH or N;

p is 1, 2 or 3;

q is 0, 1 or 2;

X is —CR⁵R⁶—, —CHR⁵CHR⁶— or a single bond;
R⁵ and R⁶ are each independently hydrogen, halogen, cyano, C₁₋₄alkyl, C₁₋₄ haloalkyl, C₁₋₄alkoxy, or C₃₋₆cycloalkyl; or (R⁵ and R⁶), and/or (R⁵ and Y), together with the atom(s) to which they are attached, form a fused C₃₋₈cycloalkyl ring, and said ring is optionally substituted with halogen, C₁₋₄ haloalkyl and C₁₋₄ alkyl;

Y and Z are each independently selected from hydrogen, halogen, C₁₋₈ alkyl, C₂₋₈ alkenyl, C₂₋₈ alkynyl, C₁₋₈ haloalkyl, C₃₋₈ cycloalkyl, heterocyclyl, aryl, or heteroaryl; or Z and Y, together with the atom(s) to which they are attached, form a bridged cyclic or heterocyclic ring optionally substituted with a substituent selected from halogen, C₁₋₄ haloalkyl, C₁₋₄ alkyl and C₁₋₄alkoxy;

Ring A is a monocyclic or bicyclic aromatic hydrocarbon ring or a monocyclic or bicyclic aromatic heterocyclic ring, each having 5- to 10-ring members; and Ring A is optionally substituted with at least one substituent R⁷ as long as valence and stability permit;

E₁, E₂, E₃ and E₄ are each independently selected from CR³ or N;

R³ is each independently selected from hydrogen, halogen, cyano, C₁₋₄ alkyl, C₃₋₈ cycloalkyl, C₂₋₈ alkenyl, C₂₋₈ alkynyl, C₁₋₄ haloalkyl, heterocyclyl, aryl, heteroaryl, —C(O)NR¹R², nitro, —C(O)OR¹, —C(O)R¹, —OR¹, —SR¹, —NR¹R², —SO₂R¹, —SO₂NR¹R², —SOR¹, —NR¹SO₂R², —NR¹SOR², —NR¹C(O)OR² or —NR¹C(O)R², wherein said C₁₋₄ alkyl, C₂₋₈ alkenyl, C₂₋₈ alkynyl, C₁₋₈ haloalkyl, C₃₋₈ cycloalkyl, heterocyclyl, aryl, and heteroaryl are each independently optionally substituted with 1 or 2 substituents R¹⁰;

R¹ and R² are each independently H, C₁₋₈ alkyl, C₃₋₈ cycloalkyl, heterocyclyl, aryl, or heteroaryl, wherein said C₁₋₈ alkyl, C₃₋₈ cycloalkyl, heterocyclyl, aryl, and heteroaryl are each independently optionally substituted with 1 or 2 substituents R¹⁰, or R¹ and R², together with the nitrogen atom to which they are attached, form a ring comprising 0, 1, 2, 3 or 4 additional heteroatoms selected from —NH, —O—, —S—, —SO— or —SO₂—, and said ring is optionally substituted with at least one substituent R¹⁰;

provided that at least one of E₁, E₂, E₃ and E₄ is CR³, wherein R³ is —C(O)NR¹R², wherein R¹ and R² are defined above;

alternatively, two adjacent R³, if present, together with the atom(s) to which they are attached, form a lactam ring, said ring comprising, in addition to the nitrogen atom forming the tactual ring, 0, 1 or 2 additional heteroatoms independently selected from nitrogen, oxygen or sulfur, and said ring is optionally substituted with halogen, C₁₋₄ haloalkyl and C₁₋₄ alkyl;

R⁷ is independently selected from hydrogen, halogen, C₁₋₈ alkyl, C₇₋₈ alkenyl, C₂₋₈ alkynyl, C₁₋₈ haloalkyl, C₃₋₈ cycloalkyl, heterocyclyl, aryl, or heteroaryl, wherein said C₁₋₈ alkyl, C₂₋₈ alkenyl, C₂₋₈ alkynyl, C₁₋₈ haloalkyl, C₃₋₈ cycloalkyl, heterocyclyl, aryl, and heteroaryl are each independently optionally substituted with 1 or 2 substituents R¹⁰;

R¹⁰, at each occurrence, is independently hydrogen, halogen, C₁₋₈ haloalkyl, C₁₋₈ alkyl, C₇₋₈ alkenyl, C₂₋₈ alkynyl, C₃₋₈ cycloalkyl, aryl, heteroaryl, heterocyclyl, oxo, —C₁₋₄alkyl-NR^{a}R^{b}, —CN, —OR^{a}, —NR^{a}R^{b}, —C(O)R^{a}, —C(O)OR¹, —CONR^{a}R^{b}, —C(═NR^{a})NR^{b}R^{c}, nitro, —NR^{a}C(O)R^{b}, NR^{a}C(O)NR^{a}R^{b}, —NR^{a}C(O)OR^{b}, —SO₂R^{a}, —NR^{a}SO₂NR^{b}R^{c}, —NR^{a}SOR^{b} or —NR^{a}SO₂R^{b}, wherein said C₁₋₈ alkyl, C₁₋₈ haloalkyl, C₃₋₈ cycloalkyl, aryl, heteroaryl, or heterocyclyl group are each independently optionally substituted by one, two or three substituents selected from halo, hydroxyl, C₁₋₄ alkyl, C₁₋₄ alkyloxy, C₁₋₄ haloalkyl, and C₁₋₄ haloalkyloxy, wherein R^{a}, R^{b}, and R^{c} are each independently selected from H, C₁₋₄ haloalkyl, C₁₋₄ alkyl, C₂₋₄ alkenyl, C₂₋₄ alkynyl, C₃₋₆ cycloalkyl, heterocyclyl, aryl, and heteroaryl, each of which is optionally substituted by one or more halogen, C₁₋₄ haloalkyl and C₁₋₄ alkyl, or (R^{a} and R^{b}), and/or (R^{b} and R^{c}) together with the atom(s) to which they are attached, form a ring selected from a heterocyclyl or heteroaryl ring optionally substituted by halogen, C₁₋₄ haloalkyl or C₁₋₄ alkyl.

Also disclosed herein is a pharmaceutical composition comprising at least one pharmaceutically acceptable excipient and a compound selected from compounds of Formula (I), or a stereoisomer thereof, or a pharmaceutically acceptable salt thereof disclosed herein.

Also disclosed herein is a method of treating cancer responsive to inhibition of IDO and/or TDO comprising administering to a subject in need of treating for such cancer an amount of a compound selected from compounds of Formula (I), or a stereoisomer thereof, or a pharmaceutically acceptable salt thereof disclosed herein effective to treat the cancer.

Also disclosed herein is a use of a compound selected from compounds of Formula (I), or a stereoisomer thereof, or a pharmaceutically acceptable salt thereof disclosed herein in manufacture of a medicament for treatment of the disorders or diseases above.

Also disclosed herein is a use of a compound selected from compounds of Formula (I), or a stereoisomer thereof, or a pharmaceutically acceptable salt thereof disclosed herein in manufacture of a medicament for inhibition of IDO and/or TDO.

Also disclosed herein is a use of a compound selected from compounds of Formula (I), or a stereoisomer thereof, or a pharmaceutically acceptable salt thereof disclosed herein in the manufacture of a medicament for treating cancer.

### DETAILED DESCRIPTION OF THE INVENTION

As used herein, the following words, phrases and symbols are generally intended to have the meanings as set forth below, except to the extent that the context in which they are used indicates otherwise.

The following abbreviations and terms have the indicated meanings throughout:

The phrase “a” or “an” entity as used herein refers to one or more of that entity. For example, a compound refers to one or more compounds or at least one compound. For another example, “ . . . substituted with a substituent . . . ” means that one or more substituents are substituted as long as valence and stability permit. As such, the terms “a” (or “an”), “one or more”, and “at least one” can be used interchangeably herein.

The term “alkyl” herein refers to a hydrocarbon group selected from linear and branched saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “alkyloxy” herein refers to an alkyl group as defined above bonded to oxygen, represented by —O-alkyl. Examples of an alkyloxy, e.g., C₁₋₆ alkyloxy or C₁₋₄ alkyloxy includes, but not limited to, methoxy, ethoxyl, isopropoxy, propoxy, n-butoxy, tert-butoxy, pentoxy and hexoxy and the like.

The term “haloalkyl” herein refers to an alkyl group in which one or more hydrogen is/are replaced by one or more halogen atoms such as fluoro, chloro, bromo, and iodo. Examples of the haloalkyl include C₁₋₆haloalkyl or C₁₋₄haloalkyl, but not limited to F₃C—, ClCH₂—, CF₃CH₂—, CF₃CCl₂—, and the like.

The term “alkenyl” herein refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkynyl” herein refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “cycloalkyl” herein refers to a hydrocarbon group selected from saturated and partially unsaturated cyclic hydrocarbon groups, comprising monocyclic and polycyclic bicyclic and tricyclic) groups. For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms, Examples of the monocyclic cycloalkyl group include cyclopropyl, cyclobutyl, cyclopentyl, 1-1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. In particular, Examples of the saturated monocyclic cycloalkyl group, e.g., C₃₋₈ cycloalkyl, include, but not limited to cyclopropyl, cyclobutyl, cyclopentyl, cyclohexyl, cycloheptyl, and cyclooctyl groups. Examples of the bicyclic cycloalkyl groups include those having from 7 to 12 ring atoms arranged as a bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems, such as

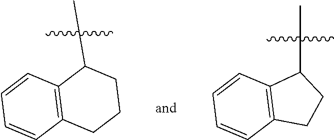 [SMILES: *C1CCCc2ccccc21 ; *C1CCc2ccccc21]

wherein the wavy lines indicate the points of attachment. The ring may be saturated or have at least one double bond (i.e. partially unsaturated), but is not fully conjugated, and is not aromatic, as aromatic is defined herein.

The term “aryl” used alone or in combination with other terms refers to a group selected from:
5- and 6-membered carbocyclic aromatic rings, for example, phenyl;
bicyclic ring systems such as 7 to 12 membered bicyclic ring systems wherein at least one ring is carbocyclic and aromatic, selected, for example, from naphthalene, and indane; and
tricyclic ring systems such as 10 to 15 membered tricyclic ring systems wherein at least one ring is carbocyclic and aromatic, for example, fluorene.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, for example, but not limited to, phenyl, naphth-1-yl, naphth-2-yl, anthracenyl, phenanthrenyl rings, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “halogen” or “halo” herein refers to F, Cl, Br or I.

The term “heteroaryl” herein refers to a group selected from:

5- to 7-membered aromatic, monocyclic rings comprising at least one heteroatom, for example, from 1 to 4, or, in some embodiments, from 1 to 3, heteroatoms, selected from N, O, and S, with the remaining ring atoms being carbon;

8- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and

11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

The terms “aromatic heterocyclic ring” and “heteroaryl” are used interchangeable throughout the disclosure herein. In some embodiments, a monocyclic or bicyclic aromatic heterocyclic ring has 5- to 10-ring forming members with 1, 2, 3, or 4 heteroatom ring members independently selected from nitrogen, sulfur and oxygen and the remaining ring members being carbon. In some embodiments, the monocyclic or bicyclic aromatic heterocyclic ring is a monocyclic or bicyclic ring comprising 1 or 2 heteroatom ring members independently selected from nitrogen, sulfur and oxygen. In some embodiments, the monocyclic or bicyclic aromatic heterocyclic ring is a 5- to 6-membered heteroaryl ring, which is monocyclic and which has 1 or 2 heteroatom ring members independently selected from nitrogen, sulfur and oxygen. In some embodiments, the monocyclic or bicyclic aromatic heterocyclic ring is a 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl (such as 1,2,3-thiadiazolyl, 1,2,4-thiadiazolyl, or 1,3,4-thiadiazolyl), tetrazolyl, thienyl (such as thien-2-yl, thien-3-yl), triazinyl, benzothienyl, furyl or furanyl, benzofuryl, benzoimidazolyl, indolyl, isoindolyl, indolinyl, oxadiazolyl (such as 1,2,3-oxadiazolyl, 1,2,4-oxadiazolyl, or 1,3,4-oxadiazolyl), phthalazinyl, pyrazinyl, pyridazinyl, pyrrolyl, triazolyl (such as 1,2,3-triazolyl, 1,2,4-triazolyl, or 1,3,4-triazolyl), quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl (such as furazan-2-yl, furazan-3-yl), benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

The term “heterocyclic” or “heterocycle” or “heterocyclyl” herein refers to a ring selected from 4- to 12-membered monocyclic, bicyclic and tricyclic, saturated and partially unsaturated rings comprising at least one carbon atoms in addition to at least one heteroatom, such as from 1-4 heteroatoms, further such as from 1-3, or further such as 1 or 2 heteroatoms, selected from oxygen, sulfur, and nitrogen. In some embodiments, a heterocyclyl group is 4- to 7-membered monocyclic ring with one heteroatom selected from N, O and S. “Heterocycle” herein also refers to a 5- to 7-membered heterocyclic ring comprising at least one heteroatom selected from N, O, and S fused with 5-, 6-, and for 7-membered cycloalkyl, carbocyclic aromatic or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl. “Heterocycle” herein also refers to an aliphatic spirocyclic ring comprising at least one heteroatom selected from N, O, and S, provided that the point of attachment is at the heterocyclic ring. The rings may be saturated or have at least one double bond (i.e. partially unsaturated). The heterocycle may be substituted with oxo. The point of the attachment may be carbon or heteroatom in the heterocyclic ring. A heterocycle is not a heteroaryl as defined herein.

Examples of the heterocycle include, but not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. A substituted heterocycle also includes a ring system substituted with one or more oxo moieties, such as piperidinyl N-oxide, morpholinyl-N-oxide, 1-oxo-1-thiomorpholinyl and 1,1-dioxo-1-thiomorpholinyl.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6] and [6,6] ring systems as mentioned above; a fused bicyclic aryl ring such as 7 to 12 membered bicyclic aryl ring systems as mentioned above, a fused tricyclic aryl ring such as 10 to 15 membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

Compounds disclosed herein may contain an asymmetric center and may thus exist as enantiomers. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and for pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or MosheR^{a}s acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” include salts of at least one compound of Formula (I), and salts of the stereoisomers of at least one compound of Formula (I), such as salts of enantiomers, and/or salts of diastereomers.

“Treating”, “treat” or “treatment” or “alleviation” refers to administering at least one compound and/or at least one stereoisomer thereof, and/or at least one pharmaceutically acceptable salt thereof disclosed herein to a subject in recognized need thereof that has, for example, cancer.

The term “effective amount” refers to an amount of at least one compound and/or at least one stereoisomer thereof, and/or at least one pharmaceutically acceptable salt thereof disclosed herein effective to “treat” as defined above, a disease or disorder in a subject.

The term “at least one substituent” disclosed herein includes, for example, from 1 to 4, such as from 1 to 3, further as 1 or 2, substituents, provided that valence and stability permit. For example, “at least one substituent R⁷” disclosed herein includes from 1 to 4, such as from 1 to 3, further as 1 or 2, substituents selected from the list of R⁷ as disclosed herein; and “at least one substituent R¹⁰” disclosed herein includes from 1 to 4, such as from 1 to 3, further as 1 or 2, substituents selected from the list of R¹⁰ as disclosed herein.

In the first aspect, disclosed herein is a compound of Formula (I)

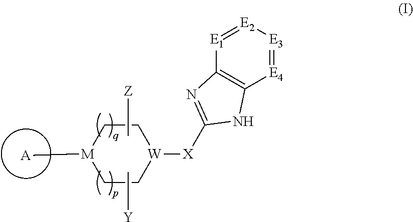

or a stereoisomer thereof, or a pharmaceutically acceptable salt thereof,

wherein:

M is CH or N;

W is CH or N;

p is 1, 2 or 3;

g is 0, 1 or 2;

X is —CR⁵R⁶—, —CHR⁵CHR⁶— or a single bond;
R⁵ and R⁶ are each independently hydrogen, halogen, cyano, C₁₋₄alkyl, C₁₋₄ haloalkyl, C₁₋₄alkoxy, or C₃₋₆cycloalkyl; or (R⁵ and R⁶), and/or (R⁵ and Y), together with the atom(s) to which they are attached, form a fused C₃₋₈cycloalkyl ring, and said ring is optionally substituted with halogen, C₁₋₄ haloalkyl and C₁₋₄ alkyl;

Y and Z are each independently selected from hydrogen, halogen, C₁₋₈ alkyl, C₂₋₈ alkenyl, C₂₋₈ alkynyl, C₁₋₈ haloalkyl, C₃₋₈ cycloalkyl, heterocyclyl, aryl, or heteroaryl; or Z and Y, together with the atom(s) to which they are attached, form a bridged cyclic or heterocyclic ring optionally substituted with a substituent selected from halogen, C₁₋₄ haloalkyl, C₁₋₄ alkyl and C₁₋₄alkoxy;

Ring A is a monocyclic or bicyclic aromatic hydrocarbon ring or a monocyclic or bicyclic aromatic heterocyclic ring, each having 5- to 10-ring members; and Ring A is optionally substituted with at least one substituent R⁷ as long as valence and stability permit;

E₁, E₂, E₃ and E₄ are each independently selected from CR³ or N;

R³ is each independently selected from hydrogen, halogen, cyano, C₁₋₈ alkyl, C₃₋₈ cycloalkyl, C₂₋₈ alkenyl, C₂₋₈ alkynyl, C₁₋₈ haloalkyl, heterocyclyl, aryl, heteroaryl, —C(O)NR¹R², nitro, —C(O)OR¹, —C(O)R¹, —OR¹, —SR¹, —NR¹R², —SO₂R¹, —SO₂NR¹R², —SOR¹, —NR¹SO₂R², —NR¹SOR², —NR¹C(O)OR² or —NR¹C(O)R², wherein said C₁₋₈ alkyl, C₂₋₈ alkenyl, C₂₋₈ alkynyl, C₁₋₈ haloalkyl, C₃₋₈ cycloalkyl, heterocyclyl, aryl, and heteroaryl are each independently optionally substituted with 1 or 2 substituents R¹⁰;

R¹ and R² are each independently H, C₁₋₈ alkyl, C₃₋₈ cycloalkyl, heterocyclyl, aryl, or heteroaryl, wherein said C₁₋₈ alkyl, C₃₋₈ cycloalkyl, heterocyclyl, aryl, and heteroaryl are each independently optionally substituted with 1 or 2 substituents R¹⁰, or R¹ and R², together with the nitrogen atom to which they are attached, form a ring comprising 0, 1, 2, 3 or 4 additional heteroatoms selected from —NH, —O—, —S—, —SO— or —SO₂—, and said ring is optionally substituted with at least one substituent R¹⁰;

provided that at least one of E₁, E₂, E₃ and E₄ is CR³, wherein R³ is —C(O)NR¹R², wherein R¹ and R² are defined above;

alternatively, two adjacent R³, if present, together with the atom(s) to which they are attached, form a lactam ring, said ring comprising, in addition to the nitrogen atom forming the lactam ring, 0, 1 or 2 additional heteroatoms independently selected from nitrogen, oxygen or sulfur, and said ring is optionally substituted with halogen, C₁₋₄ haloalkyl and C₁₋₄ alkyl;

R⁷ is independently selected from hydrogen, halogen, C₁₋₈ alkyl, C₂₋₈ alkenyl, C₂₋₈ alkynyl, C₁₋₈ haloalkyl, C₃₋₈ cycloalkyl, heterocyclyl, aryl, or heteroaryl, wherein said C₁₋₈ alkyl, C₂₋₈ alkenyl, C₂₋₈ alkynyl, C₁₋₈ haloalkyl, C₃₋₈ cycloalkyl, heterocyclyl, aryl, and heteroaryl are each independently optionally substituted with 1 or 2 substituents R¹⁰;

R¹⁰, at each occurrence, is independently hydrogen, halogen, C₁₋₈ haloalkyl, C₁₋₈ alkyl, C₂₋₈ alkenyl, C₂₋₈ alkynyl, C₃₋₈ cycloalkyl, aryl, heteroaryl, heterocyclyl, oxo, —C₁₋₄ alkyl-NR^{a}R^{b}, —CN, —OR^{a}, —NR^{a}R^{b}, —C(O)R^{a}, —C(O)OR^{a}, —CONR^{a}R^{b}, —C(═NR^{a})NR^{b}R^{c}, nitro, —NR^{a}C(O)R^{b}, —NR^{a}C(O)NR^{a}R^{b}, —NR^{a}C(O)OR^{b}, —SO₂R^{a}, —NR^{a}SO₂NR^{b}R^{c}, —NR^{a}SOR^{b} or —NR^{a}SO₂R^{b}, wherein said C₁₋₈ alkyl, C₁₋₈ haloalkyl, C₃₋₈ cycloalkyl, aryl, heteroaryl, or heterocyclyl group are each independently optionally substituted by one, two or three substituents selected from halo, hydroxyl, C₁₋₄ alkyl, C₁₋₄ alkyloxy, C₁₋₄ haloalkyl, and C₁₋₄ haloalkyloxy, wherein R^{a}, R^{b}, and R^{c} are each independently selected from H, C₁₋₄ haloalkyl, C₁₋₄ alkyl, C₂₋₄ alkenyl, C₂₋₈ alkynyl, C₃₋₈ cycloalkyl, heterocyclyl, aryl, and heteroaryl, each of which is optionally substituted by one or more halogen, C₁₋₄ haloalkyl and C₁₋₄ alkyl, or (R^{a} and R^{b}), and/or (R^{b} and R^{c}) together with the atom(s) to which they are attached, form a ring selected from a heterocyclyl or heteroaryl ring optionally substituted by halogen, C₁₋₄ haloalkyl or C₁₋₄ alkyl.

In one embodiment of the first aspect, p is 1, and q is 1. In another embodiment, p is 1 and q is 0. In one embodiment of the first aspect, W is N and M is CH. In another embodiment, W and M are both N. In further another embodiment, W and M are both CH. In yet further embodiment, W is CH and M is N.

In one embodiment, Z and Y are each independently selected from hydrogen, halogen, C₁₋₈ alkyl, C₂₋₈ alkenyl, C₂₋₈ alkynyl, C₁₋₈ haloalkyl, C₃₋₈ cycloalkyl, heterocyclyl, aryl, or heteroaryl.

In one embodiment, Z and Y together with the atoms to which they are attached, form a bridged bicyclic ring optionally substituted with a substituent selected from halogen, C₁₋₄ haloalkyl, C₁₋₈ alkyl and C₁₋₄alkoxy. Preferably, Z and Y, together with the atoms to which they are attached, form a bridged bicyclic ring selected from bicyclo[2.2.1]heptyl (e.g., bicyclo[2.2.1]heptan-2-yl), born-2-yl, bicyclo[2.2.2]octyl, bicyclo[3.2.1]octyl, bicyclo[3.3.1]nonyl, or bicyclo[3.3.2.]decyl. More preferably, the bridged bicyclic ring is bicyclo[2.2.1]heptyl or bicyclo[2.2.2]octyl.

Preferably, the

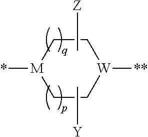

moiety is

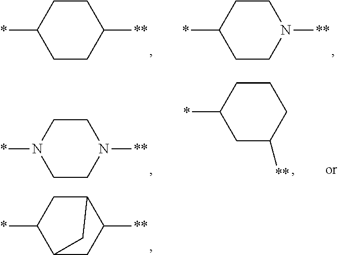

wherein * indicates a link to the ring A, and ** indicates a link to X.

In one embodiment, X is —CR⁵R⁶—, wherein R⁵ is C₁₋₄alkyl, C₁₋₄haloalkyl, C₁₋₄alkoxy, or C₃₋₆cycloalkyl, and R⁶ is hydrogen.

In another embodiment, X is —CR⁵R⁶—, wherein R⁵ is C₁₋₄alkyl, C₁₋₄haloalkyl, C₁₋₄alkoxy, or C₃₋₆cycloalkyl, and R⁶ is hydrogen, and the

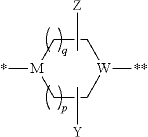

moiety is

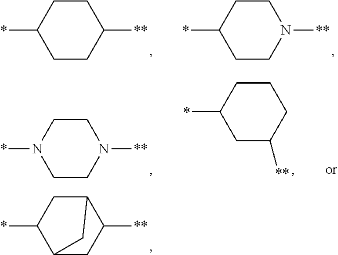

wherein * indicates a link to the ring A, and ** indicates a link to X. In a further preferred embodiment, R⁵ is methyl, trifluoromethyl, methoxy, or cyclopropyl, and R⁶ is hydrogen. or R⁵ and R⁶), and/or (R⁵ and Y), together with the atoms to which they are attached, form a fused C₃₋₈cycloalkyl ring, and said ring is optionally substituted with halogen, C₁₋₄ haloalkyl and C₁₋₈ alkyl, and said ring is selected from cyclopropyl, cyclobutyl, cyclopentyl and cyclohexyl. Cyclopropyl is preferred.

In some embodiment, ring A is phenyl or naphthalenyl ring. In some embodiment, ring A is a monocyclic or bicyclic aromatic heterocyclic ring having 5- to 10-ring members comprising 1, 2, 3, or 4 heteroatoms selected from O, S, and N.

In some embodiment, ring A is a monocyclic aromatic heterocyclic ring having 5- to 6-ring members comprising 1 or 2 heteroatoms selected from O, S, and N. In other embodiments, ring A is pyridinyl, furanyl, pyrazinyl, pyrimidinyl, pyridazinyl, pyrrolyl, thienyl, triazinyl, or pyrazolyl. In some preferred embodiments, ring A is pyridinyl or furanyl.

In some embodiment, ring A is a bicyclic aromatic heterocyclic ring having 8- to 10-ring members comprising 1 or 2 or 3 heteroatoms selected from O, S, and N. In another embodiment, ring A is cinnolinyl, benzothienyl, benzofuryl, benzoimidazolyl, indolyl, isoindolyl, indolinyl, phthalazinyl, quinolinyl, isoquinolinyl, pyrrolopyridinyl, pyrazolopyridinyl, benzodioxolyl, benzoxazolyl, pteridinyl, purinyl, benzofurazanyl, benzothiophenyl, benzothiazolyl, benzoxazolyl, quinazolinyl, quinoxalinyl, naphthyridinyl, furopyridinyl, benzothiazolyl, or indazolyl. In some preferred embodiments, ring A is benzothiophenyl (such as benzo[b]thiophen-2-yl, benzo[b]thiophen-3-yl, benzo[b]thiophen-5-yl, or benzo[b]thiophen-6-yl) or quinolinyl (such as quinolin-2-yl, quinolin-3-yl, quinolin-4-yl, quinolin-5-yl, quinolin-6-yl, quinolin-7-yl, quinolin-8-yl) or benzodioxolyl (such as benzo[d][1,3]dioxol-5-yl).

In one embodiment, ring A is optionally substituted with one substituent R⁷ which is independently hydrogen, halogen, C₁₋₈ alkyl, C₂₋₈ alkenyl, C₂₋₈ alkynyl, C₁₋₈ haloalkyl, C₃₋₈ cycloalkyl, heterocyclyl, aryl, or heteroaryl.

In one preferred embodiment, ring A is quinolinyl (such as quinolin-2-yl, quinolin-3-yl, quinolin-4-yl, quinolin-5-yl, quinolin-6-yl, quinolin-7-yl, quinolin-8-yl) optionally substituted with halogen or C₁₋₈haloalkyl. More preferably, ring A is 6-fluoroquinolin-4-yl or 8-fluoro-quinolin-5-yl.

In one embodiment, the combination of E₁, E₂, E₃ and E₄ are as follows:

(a) E₁ is CR³, E₂ is —CC(O)NR¹R², E₃ is CR³, and E₄ is CR³;

(b) E₁ is CR³, E₂ is CR³, E₃ is —CC(O)NR¹R², and E₄ is CR³;

(c) E₁ is N, E₂ is —CC(O)NR¹R², E₃ is CR³, and E₄ is CR³;

(d) E₁ is N, E₂ is CR³, E₃ is —CC(O)NR¹R², and E₄ is CR³;

(e) E₁ is CR³, E₂ is —CC(O)NR¹R², E₃ is CR³, and E₄ is N;

(f) E₁ is CR³, E₂ is CR³, E₃ is —CC(O)NR¹R², and E₄ is N;

(g) E₁ is N, E₂ is —CC(O)NR¹R², E₃ is CR³, and E₄ is N;

(h) E₁ is N, E₂ is CR³, E₃ is —CC(O)NR¹R², and E₄ is N;

(i) E₁ is CR³, E₂ is —CC(O)NR¹R², E₃ is N, and E₄ is CR³;

(j) E₁ is CR³, E₂ is N, E₃ is —CC(O)NR¹R², and E₄ is CR³;

(k) E₁ is N, E₂ is —CC(O)NR¹R², E₃ is N, and E₄ is CR³;

(l) E₁ is CR³, E₂ is N, E₃ is —CC(O)NR¹R², and E₄ is N;

wherein R¹, R², and R³ are defined as for formula (I).

In some embodiment, R³ is halogen, or C₁₋₄ alkyl (more preferably methyl, ethyl).

In some embodiment, R¹ and R² are each independently H, C₁₋₈ alkyl (more preferably methyl, ethyl), C₃₋₈cycloalkyl (more preferably cyclopropyl, cyclobutyl, cyclohexyl), aryl (e.g., phenyl), heterocyclyl or heteroaryl, wherein said C₁₋₈ alkyl, C₃₋₈ cycloalkyl, or aryl are each independently optionally substituted with 1 or 2 substituents R¹⁰, or R¹ and R², together with the nitrogen atom to which they are attached, form a 3-, 4-, 5-, or 6-membered saturated ring comprising 0 additional heteroatom, and said ring is optionally substituted with at least one substituent R¹⁰; preferably, R¹ and R², together with the nitrogen atom to which they are attached, form azetidin-1-yl, azetidin-1-yl, pyrrolidin-1-yl, or piperidin-1-yl).

In some embodiment, R¹ is hydrogen and R² are C₃₋₈cycloalkyl (more preferably cyclopropyl, cyclobutyl, cyclohexyl), or aryl (e.g., phenyl), wherein said C₃₋₈ cycloalkyl, or aryl are each independently optionally substituted with 1 or 2 substituents R¹⁰, wherein R¹⁰ is —OR^{a}, and R^{a} is H, C₁₋₄ haloalkyl, or C₁₋₄ alkyl.

In some embodiment, R¹ is hydrogen and R² are C₁₋₈ alkyl, wherein said C₁₋₈ alkyl is optionally substituted with 1 or 2 substituents R¹⁰, wherein R¹⁰ is C₃₋₈ cycloalkyl or heterocyclyl, said C₃₋₈ cycloalkyl or heterocyclyl is each independently optionally substituted by one, two or three substituents selected from halo, hydroxyl, C₁₋₄ alkyl, C₁₋₄ alkyloxy, C₁₋₄ haloalkyl, and C₁₋₄ haloalkyloxy.

In one embodiment, R¹ and R² independently selected from hydrogen, C₁₋₈ alkyl, C₃₋₈ cycloalkyl, C₃₋₈ cycloalkyl-alkyl, aryl, C₁₋₈alkyloxy, heterocyclyl, wherein said C₁₋₈ alkyl, C₁₋₈ cycloalkyl, aryl are each independently optionally substituted with 1 or 2 substituents R¹⁰. R¹ and R¹⁰ are independently selected from C₁₋₈ alkyl, C₁₋₈alkyloxy, C₃₋₈ cycloalkyl, wherein said C₁₋₈ alkyl, C₁₋₈alkyloxy, C₃₋₈ cycloalkyl are each independently optionally substituted with halo, hydroxyl, C₁₋₄ alkyl.

In yet further embodiment, R¹ is hydrogen or methyl, R² is independently selected from methyl, ethyl, n-propyl, isopropyl, n-butyl, isobutyl, s-butyl, t-butyl, methoxy, cyclopropyl, cyclobutyl, cyclopentyl, cyclohexyl, phenyl, methoxyethyl, hydroxycyclobutylmethyl, oxetanyl.

In one embodiment, two adjacent R³ if present, to ether with the atom(s) to which they are attached, form a lactam ring, which is

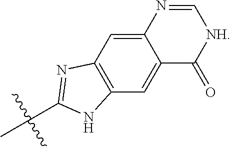 [SMILES: *c1nc2cc3nc[nH]c(=O)c3cc2[nH]1]

Specifically, the compound of Formula (I) is a compound of Formula (Ia):

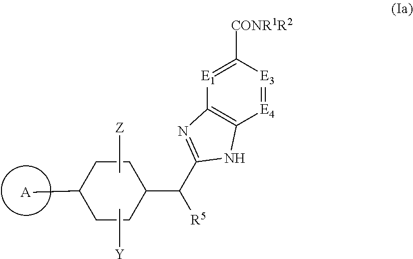

wherein the variables R¹, R², R⁵, Z, Y, E₁, E₃, E₄ and A are defined as for Formula (I).

Specifically, the compound of Formula (I) is a compound of Formula (Ib):

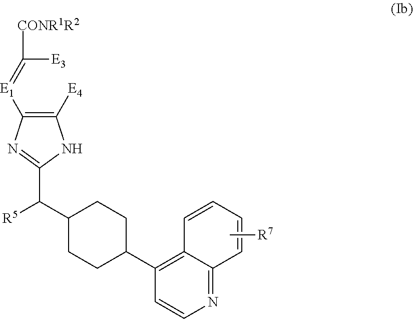

wherein R⁵ is C₁₋₄alkyl, C₁₋₄haloalkyl, C₁₋₄alkoxy, or C₃₋₆cycloalkyl; R⁷ is halogen, R¹, R², E₁, E₃ and E₄ are defined as for Formula (I).

In one embodiment, the compound disclosed herein has one of the following configurations:

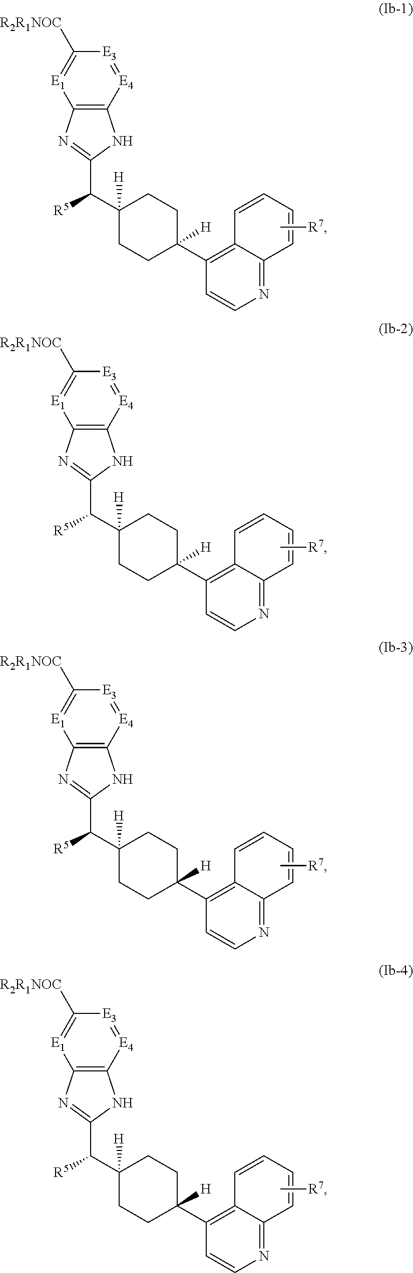

wherein R⁵ is C₁₋₄alkyl, C₁₋₄haloalkyl, C₁₋₄alkoxy, or C₃₋₆cycloalkyl; R⁷ is halogen, R¹, R², E₁, E₃ and E₄ are defined as for Formula (I).

Also disclosed herein is a compound selected from the group consisting of:

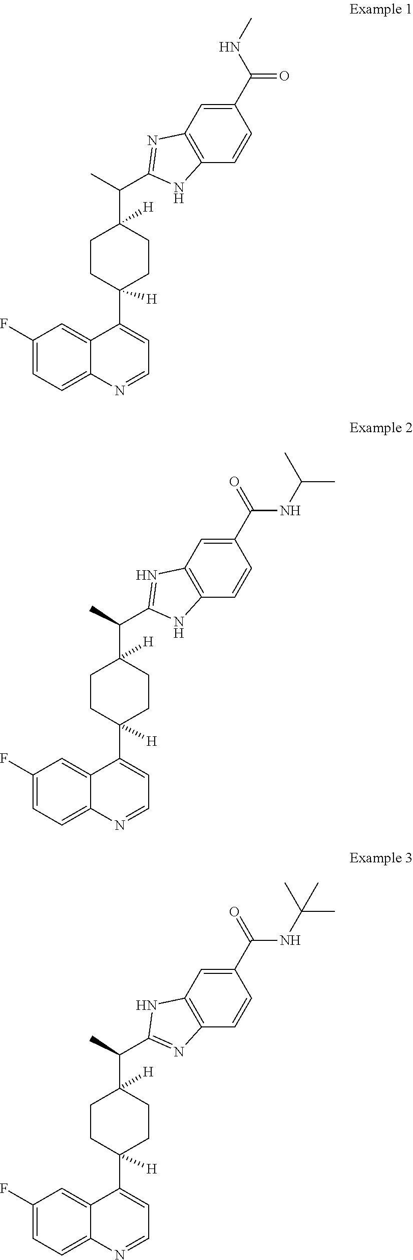 [SMILES: CNC(=O)c1ccc2[nH]c(C(C)[C@H]3CC[C@@H](c4ccnc5ccc(F)cc54)CC3)nc2c1 ; CC(C)NC(=O)c1ccc2[nH]c([C@H](C)[C@H]3CC[C@@H](c4ccnc5ccc(F)cc54)CC3)nc2c1 ; C[C@@H](c1nc2ccc(C(=O)NC(C)(C)C)cc2[nH]1)[C@H]1CC[C@@H](c2ccnc3ccc(F)cc32)CC1]

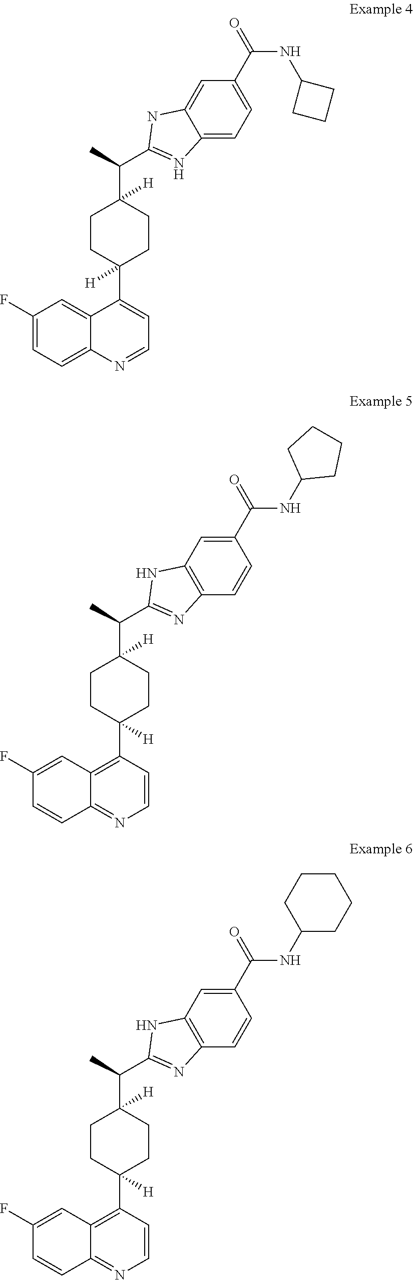 [SMILES: C[C@@H](c1nc2ccc(C(=O)NC3CCC3)cc2[nH]1)[C@H]1CC[C@@H](c2ccnc3ccc(F)cc32)CC1 ; C[C@@H](c1nc2ccc(C(=O)NC3CCCC3)cc2[nH]1)[C@H]1CC[C@@H](c2ccnc3ccc(F)cc32)CC1 ; C[C@@H](c1nc2ccc(C(=O)NC3CCCCC3)cc2[nH]1)[C@H]1CC[C@@H](c2ccnc3ccc(F)cc32)CC1]

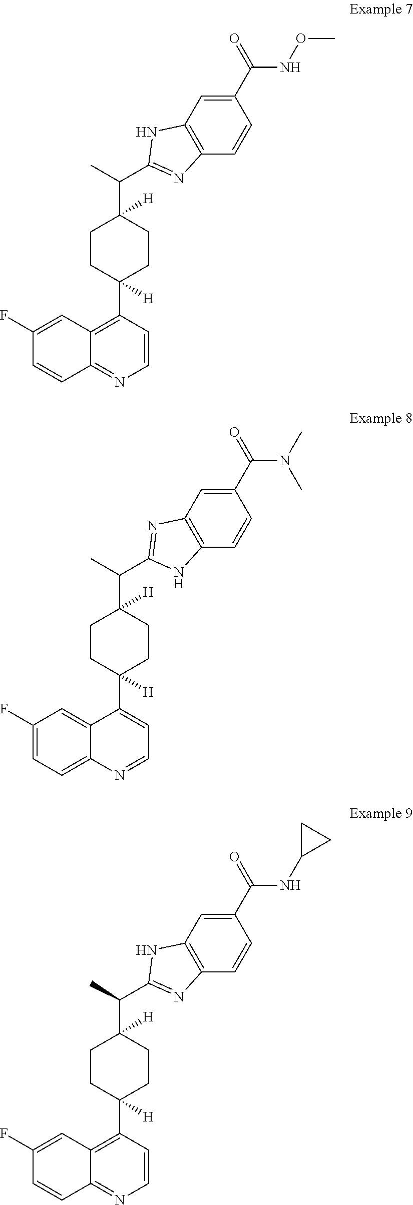 [SMILES: CONC(=O)c1ccc2nc(C(C)[C@H]3CC[C@@H](c4ccnc5ccc(F)cc54)CC3)[nH]c2c1 ; CC(c1nc2cc(C(=O)N(C)C)ccc2[nH]1)[C@H]1CC[C@@H](c2ccnc3ccc(F)cc32)CC1 ; C[C@@H](c1nc2ccc(C(=O)NC3CC3)cc2[nH]1)[C@H]1CC[C@@H](c2ccnc3ccc(F)cc32)CC1]

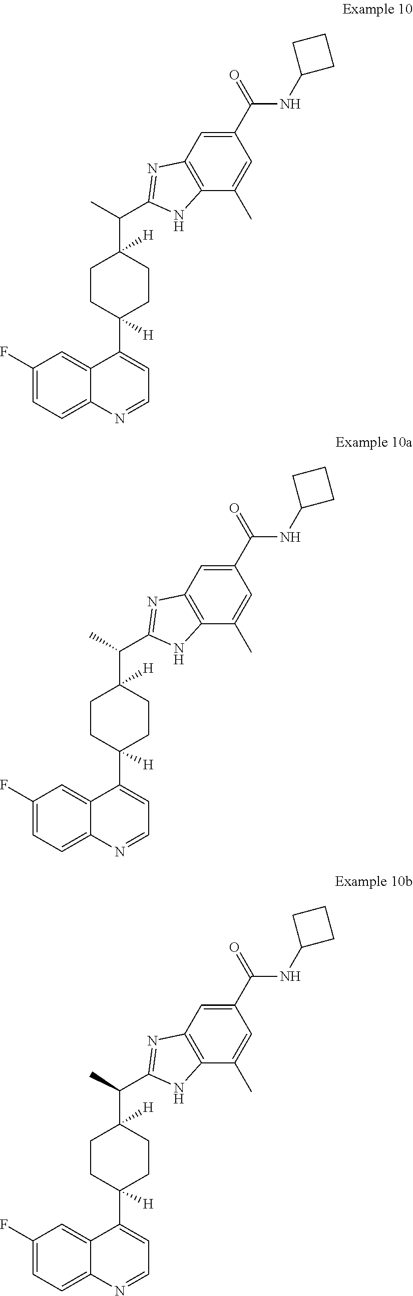 [SMILES: Cc1cc(C(=O)NC2CCC2)cc2nc(C(C)[C@H]3CC[C@@H](c4ccnc5ccc(F)cc54)CC3)[nH]c12 ; Cc1cc(C(=O)NC2CCC2)cc2nc([C@@H](C)[C@H]3CC[C@@H](c4ccnc5ccc(F)cc54)CC3)[nH]c12 ; Cc1cc(C(=O)NC2CCC2)cc2nc([C@H](C)[C@H]3CC[C@@H](c4ccnc5ccc(F)cc54)CC3)[nH]c12]

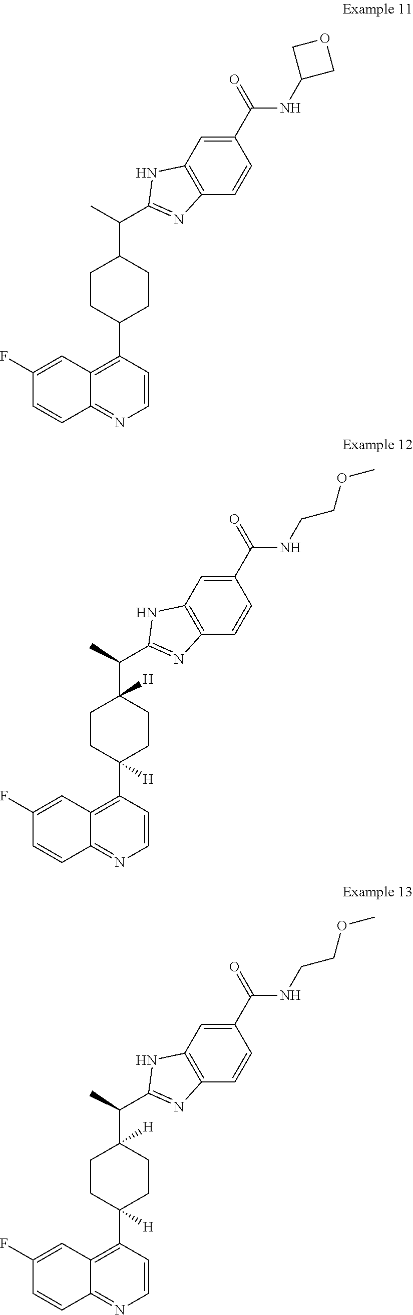 [SMILES: CC(c1nc2ccc(C(=O)NC3COC3)cc2[nH]1)C1CCC(c2ccnc3ccc(F)cc23)CC1 ; COCCNC(=O)c1ccc2nc([C@H](C)[C@H]3CC[C@H](c4ccnc5ccc(F)cc54)CC3)[nH]c2c1 ; COCCNC(=O)c1ccc2nc([C@H](C)[C@H]3CC[C@@H](c4ccnc5ccc(F)cc54)CC3)[nH]c2c1]

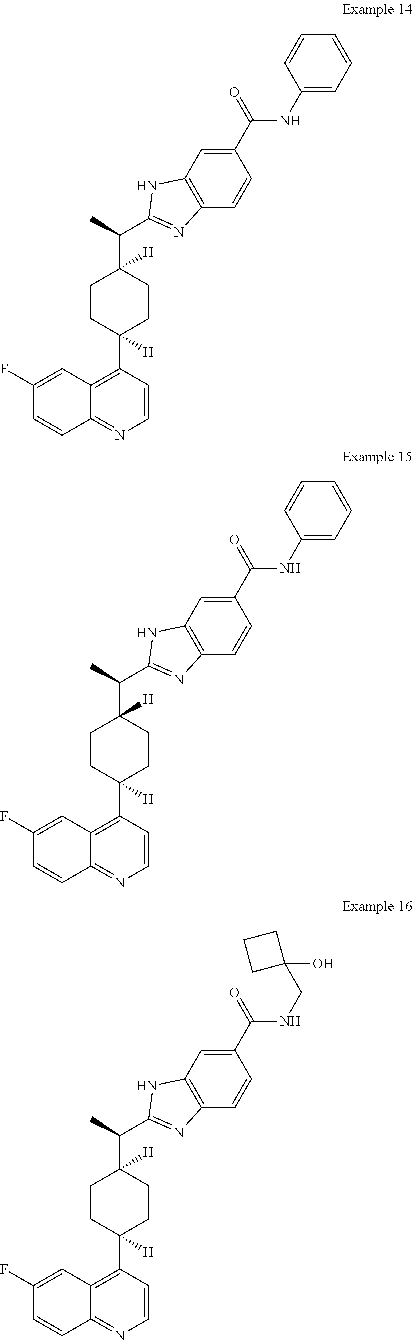 [SMILES: C[C@@H](c1nc2ccc(C(=O)Nc3ccccc3)cc2[nH]1)[C@H]1CC[C@@H](c2ccnc3ccc(F)cc32)CC1 ; C[C@@H](c1nc2ccc(C(=O)Nc3ccccc3)cc2[nH]1)[C@H]1CC[C@H](c2ccnc3ccc(F)cc32)CC1 ; C[C@@H](c1nc2ccc(C(=O)NCC3(O)CCC3)cc2[nH]1)[C@H]1CC[C@@H](c2ccnc3ccc(F)cc32)CC1]

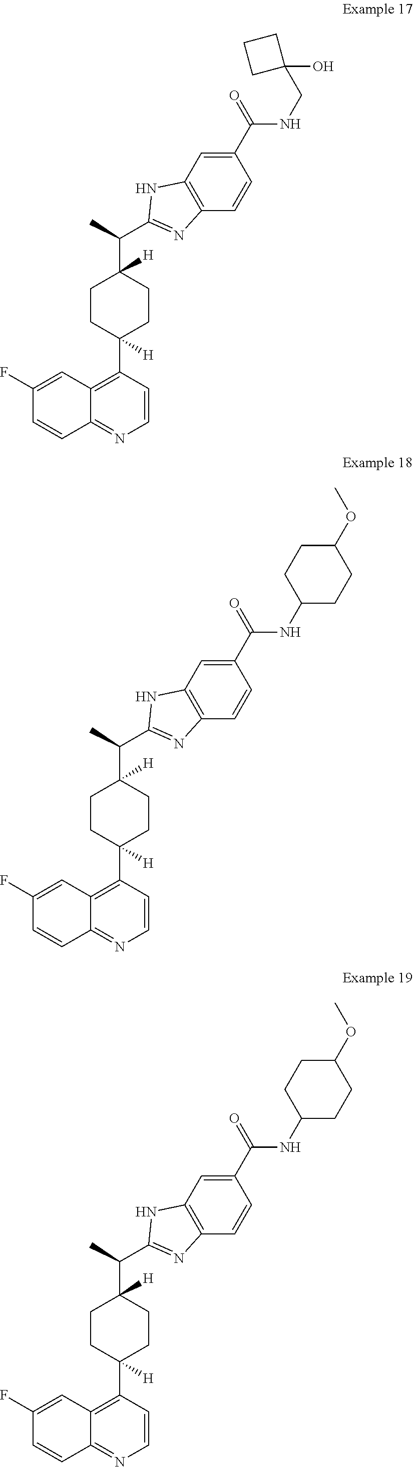 [SMILES: C[C@@H](c1nc2ccc(C(=O)NCC3(O)CCC3)cc2[nH]1)[C@H]1CC[C@H](c2ccnc3ccc(F)cc32)CC1 ; COC1CCC(NC(=O)c2ccc3nc([C@H](C)[C@H]4CC[C@@H](c5ccnc6ccc(F)cc65)CC4)[nH]c3c2)CC1 ; COC1CCC(NC(=O)c2ccc3nc([C@H](C)[C@H]4CC[C@H](c5ccnc6ccc(F)cc65)CC4)[nH]c3c2)CC1]

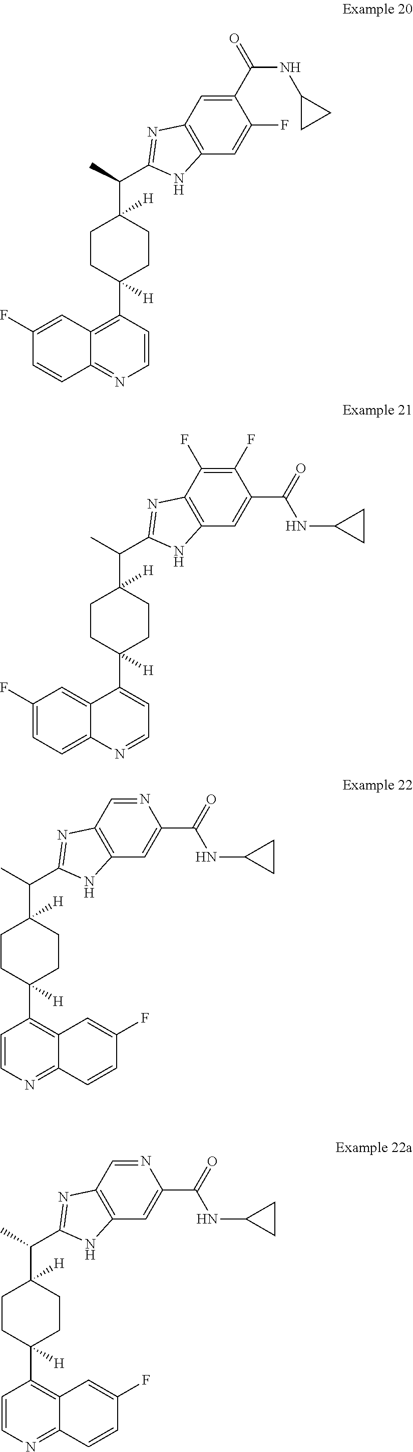

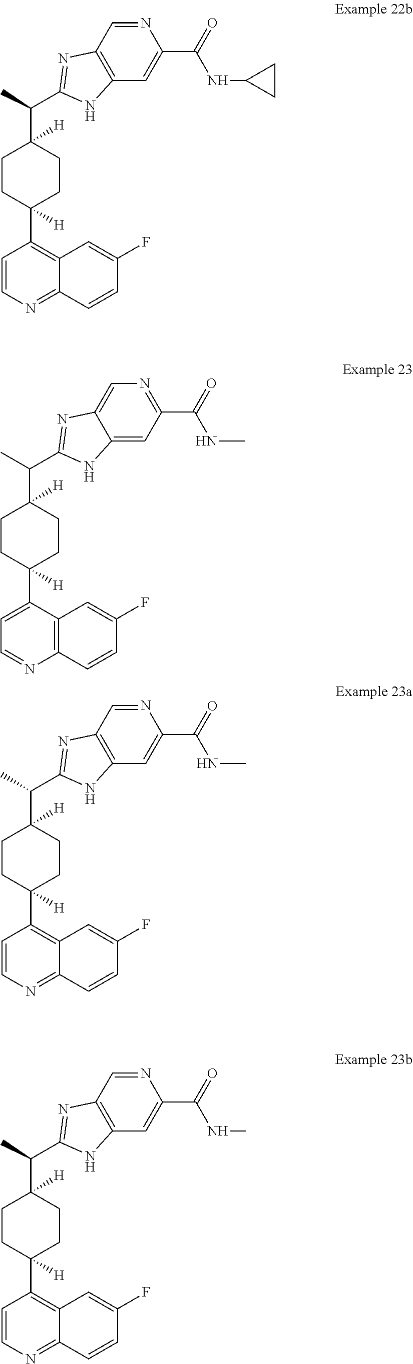

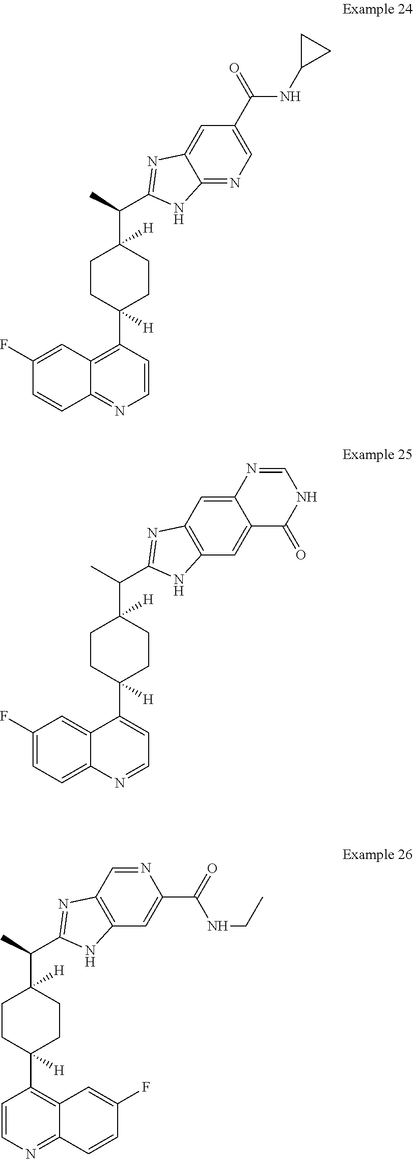 [SMILES: C[C@@H](c1nc2cc(C(=O)NC3CC3)cnc2[nH]1)[C@H]1CC[C@@H](c2ccnc3ccc(F)cc32)CC1 ; CC(c1nc2cc3nc[nH]c(=O)c3cc2[nH]1)[C@H]1CC[C@@H](c2ccnc3ccc(F)cc32)CC1 ; CCNC(=O)c1cc2[nH]c([C@H](C)[C@H]3CC[C@@H](c4ccnc5ccc(F)cc54)CC3)nc2cn1]

or a pharmaceutically acceptable salt thereof.

In the fourth aspect, disclosed herein is the process for preparing the compounds of formulas (I), (Ia), (Ib), (Ib-1), (Ib-2), (Ib-3), (Ib-4) disclosed herein.

The compounds disclosed herein, and for the pharmaceutically acceptable salts thereof, can be synthesized from commercially available starting materials taken together with the disclosure herein.

Compounds of Formula (I) including formulas (I), (Ia), (Ib), (Ib-1), (Ib-2), (Ib-3), (Ib-4) may be prepared by the exemplary processes described in the working Examples, as well as relevant published literature procedures that are used by one skilled in the art. Exemplary reagents and procedures for these reactions appear hereinafter and in the working Examples. Protection and deprotection in the processes below may be carried out by procedures generally known in the art (see, for example, Greene, T. W. et al., eds., Protecting Groups in Organic Synthesis, 3^{rd} Edition, Wiley (1999)). General methods of organic synthesis and functional group transformations are found in: Trost, B. M. et al., eds., Comprehensive Organic Synthesis: Selectivity. Strategy & Efficiency in Modern Organic Chemistry, Pergamon Press, New York, N.Y. (1991); March, J., Advanced Organic Reactions, Mechanisms, and Structure. 4^{th} Edition, Wiley & Sons, New York, N.Y. (1992); Katritzky, A. R. et al., eds., Comprehensive Organic Functional Groups Transformations, 1^{st} Edition, Elsevier Science Inc., Tarrytown, N.Y. (1995); Larock, R. C., Comprehensive Organic Transformations, VCH Publishers, Inc., New York, N.Y. (1989), and references therein.

Compounds of the invention (Ic) may be prepared according to the following schemes utilizing chemical transformations familiar to anyone of ordinary proficiency in the art of organic/medicinal chemistry. References to many of these transformations can be found in March's Advanced Organic Chemistry Reactions, Mechanisms, and Structure, Fifth Edition by Michael B. Smith and Jerry March, Wiley-Interscience, New York, 2001, or other standard texts on the topic of synthetic organic chemistry.

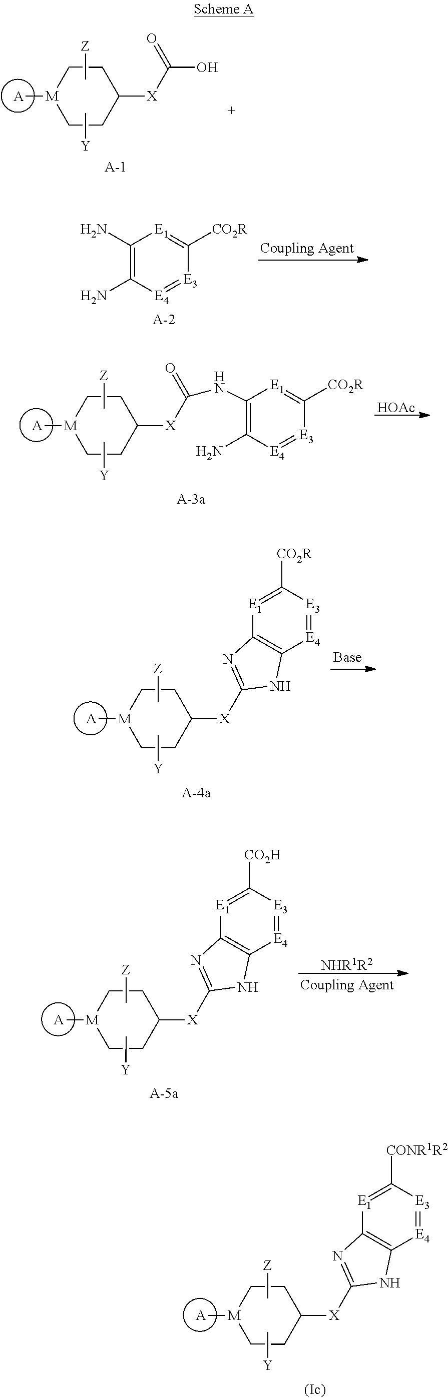

Compounds Ic can be prepared by a procedure depicted in Scheme A. The starting acid A-1 is converted into the amide A-3a through coupling with A-2. The amide A-3a can be cyclized into the A-4a by treatment with hot acetic acid. The ester A-4a can be hydrolyzed into acid A-5a through basic condition. The acid A-5a is converted into the final amide-substituted imidazo compounds (Ic).

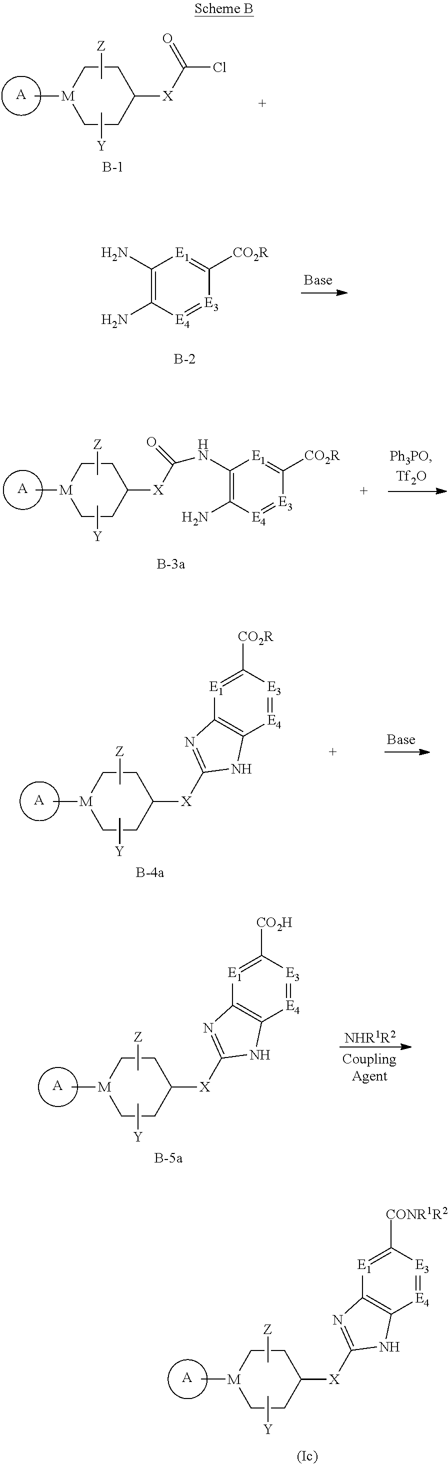

Compounds Ia can be prepared by a procedure depicted in Scheme B. The starting acid B-1 is converted into the amide B-3a through coupling with B-2. The amide B-3a can be cyclized into the B-4a by treatment with POPh₃ and Tf₂O. The ester B-4a can be hydrolyzed into acid B-5a through basic condition. The acid B-5a is converted into the final amide-substituted imidazo compounds Ic.

The syntheses of the starting acid and chloride are described in the corresponding examples in the experimental part.

In the fifth aspect, disclosed herein is a method for treating or preventing hyperproliferative disorders, such as cancer, comprising administrating to a subject, such as a mammal or human in need thereof a pharmaceutically effective amount of a compound selected from compounds of Formulas (I), (Ia), (Ib), (Ib-1), (Ib-2), (Ib-3), (Ib-4), or a stereoisomer thereof, or a pharmaceutically acceptable salt thereof disclosed herein.

Also disclosed herein is a method for treating or preventing hyperproliferative disorders, such as cancer by inhibiting IDO, comprising administrating to a subject, such as a mammal or human in need thereof a pharmaceutically effective amount of a compound selected from compounds of Formulas (I), (Ia), (Ib), (Ib-1), (Ib-2), (Ib-3), (Ib-4), or a stereoisomer thereof, or a pharmaceutically acceptable salt thereof disclosed herein.

Also disclosed herein is a method for treating or preventing cancer including but not limiting to, for example, melanomas, thyroid cancer, Barret's adenocarcinoma, breast cancer, cervical cancer, colorectal cancer, gastric cancer, lung cancer, renal carcinoma, head and neck cancer, liver cancer, stomach cancer, esophageal cancer, ovarian cancer, pancreatic cancer, prostate cancer, hematologic cancers, cancer of Biliary Tract, Non-small-cell-lung cancer, endometrium cancer, blood cancer, large intestinal colon carcinoma, histiocytic lymphoma, lung adenocarcinoma, comprising administrating to a subject, such as a mammal or human in need thereof a pharmaceutically effective amount of a compound selected from compounds of Formulas (I), (Ia), (Ib), (Ib-1), (Ib-2), (Ib-3), (Ib-4), or a stereoisomer thereof, or a pharmaceutically acceptable salt thereof disclosed herein.

Also disclosed herein is a method for treating or preventing HIV/AIDS, comprising administrating to a subject, such as a mammal or human in need thereof a pharmaceutically effective amount of a compound selected from compounds of Formulas (I), (Ia), (Ib), (Ib-1), (Ib-2), (Ib-3), (Ib-4), or a stereoisomer thereof, or a pharmaceutically acceptable salt thereof disclosed herein.

Also disclosed herein is a method for enhancing the effectiveness of an anti-retroviral therapy, comprising administrating to a subject, such as a mammal or human in need thereof an anti-retroviral agent and a pharmaceutically-effective amount of a compound selected from compounds of Formulas (I), (Ia), (Ib), (Ib-1), (Ib-2), (Ib-3), (Ib-4), or a stereoisomer thereof, or a pharmaceutically acceptable salt thereof disclosed herein.

Also disclosed herein is a method of treating cancer responsive to inhibition of IDO comprising administering to a subject, such as a mammal or human, in need of treating for the cancer a pharmaceutically-effective amount of a compound selected from compounds of Formulas (I), (Ia), (Ib), (Ib-1), (Ib-2), (Ib-3), (Ib-4), or a stereoisomer thereof, or a pharmaceutically acceptable salt thereof disclosed herein, wherein the cancer includes but not limiting to, for example, melanomas, thyroid cancer, Barret's adenocarcinoma, breast cancer, cervical cancer, colorectal cancer, gastric cancer, lung cancer, renal carcinoma, head and neck cancer, liver cancer, stomach cancer, esophageal cancer, ovarian cancer, pancreatic cancer, prostate cancer, hematologic cancers, cancer of Biliary Tract, Non-small cell lung cancer, endometrium cancer, blood cancer, large intestinal colon carcinoma, histiocytic lymphoma, lung adenocarcinoma.

Also disclosed herein is a use of a compound selected from compounds of (I), or a stereoisomer thereof, or a pharmaceutically acceptable salt thereof disclosed herein in the manufacture of a medicament for the treatment of cancer responsive to inhibition of IDO, wherein the cancer includes but not limiting to, for example, melanomas, thyroid cancer, Barret's adenocarcinoma, breast cancer, cervical cancer, colorectal cancer, gastric cancer, lung cancer, renal carcinoma, head and neck cancer, liver cancer, stomach cancer, esophageal cancer, ovarian cancer, pancreatic cancer, prostate cancer, hematologic cancers, cancer of Biliary Tract, Non-small cell lung cancer, endometrium cancer, blood cancer, large intestinal colon carcinoma, histiocytic lymphoma, lung adenocarcinoma.

Also disclosed herein is a compound selected from compounds of (I), or a stereoisomer thereof; or a pharmaceutically acceptable salt thereof disclosed herein for use in the treatment of cancer responsive to inhibition of IDO, wherein the cancer includes but not limiting to, for example, melanomas, thyroid cancer, Barret's adenocarcinoma, breast cancer, cervical cancer, colorectal cancer, gastric cancer, lung cancer, renal carcinoma, head and neck cancer, liver cancer, stomach cancer, esophageal cancer, ovarian cancer, pancreatic cancer, prostate cancer, hematologic cancers, cancer of Biliary Tract, Non-small cell lung cancer, endometrium cancer, blood cancer, large intestinal colon carcinoma, histiocytic lymphoma, lung adenocarcinoma.

The compound selected from compounds of Formulas (I), (Ia), (Ib), (Ib-1), (Ib-2), (Ib-3), (Ib-4), or a stereoisomer thereof, or a pharmaceutically acceptable salt thereof may be employed alone or in combination with at least one other therapeutic agent for treatment. In some embodiments, the compound selected from compounds of Formulas (I), (Ia), (Ib), (Ib-1), (Ib-2), (Ib-3), (Ib-4), or a stereoisomer thereof, or a pharmaceutically acceptable salt thereof can be used in combination with at least one additional therapeutic agent. The at least one additional therapeutic agent can be, for example, selected from anti-hyperproliferative, anti-cancer, and chemotherapeutic agents. The at least one compound and/or at least one pharmaceutically acceptable salt disclosed herein may be administered with the at least one other therapeutic agent in a single dosage form or as a separate dosage form. When administered as a separate dosage form, the at least one other therapeutic agent may be administered prior to, at the same time as, or following administration of the at least one compound and/or at least one pharmaceutically acceptable salt disclosed herein.

A “chemotherapeutic agent” is a chemical compound useful in the treatment of cancer, regardless of mechanism of action. Chemotherapeutic agents include compounds used in “targeted therapy” and conventional chemotherapy. Suitable chemotherapeutic agents can be, for example, selected from: agents that induce apoptosis; polynucleotides (e.g., ribozymes); polypeptides (e.g., enzymes); drugs; biological mimetics; alkaloids; alkylating agents; antitumor antibiotics; antimetabolites; hormones; platinum compounds; monoclonal antibodies conjugated with anticancer drugs, toxins, and/or radionuclides; biological response modifiers (e.g., interferons, such as IFN-a and interleukins, such as IL-2); adoptive immunotherapy agents; hematopoietic growth factors; agents that induce tumor cell differentiation (e.g., all-trans-retinoic acid); gene therapy reagents; antisense therapy reagents and nucleotides; tumor vaccines; and inhibitors of angiogenesis.

In the sixth aspect, disclosed herein is a pharmaceutical composition comprising a compound selected from compounds of Formulas (I), (Ia), (Ib), (Ib-1), (Ib-2), (Ib-3), (Ib-4) or a stereoisomer thereof, or a pharmaceutically acceptable salt thereof disclosed herein and a pharmaceutically acceptable excipient, e.g., a carrier, a diluent, or a adjuvant.

Also disclosed herein is a composition comprising a compound selected from compounds of Formulas (I), (Ia), (Ib), (Ib-1), (Ib-2), (Ib-3), (Ib-4) or a stereoisomer thereof, or a pharmaceutically acceptable salt thereof, and at least one pharmaceutically acceptable excipient.

The composition comprising a compound selected from compounds of (I), (Ia), (Ib), (Ib-1), (Ib-2), (Ib-3), (Ib-4), or a stereoisomer thereof, or a pharmaceutically acceptable salt thereof can be administered in various known manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art.

The compound selected from Formulas (I), (Ia), (Ib), (Ib-1), (Ib-2), (Ib-3), (Ib-4), or a stereoisomer thereof, or a pharmaceutically acceptable salt thereof can be administered orally in solid dosage forms, such as capsules, tablets, troches, dragées, granules and powders, or in liquid dosage forms, such as elixirs, syrups, emulsions, dispersions, and suspensions. The compound selected from compounds of Formulas (I), (Ia), (Ib), (Ib-1), (Ib-2), (Ib-3), (Ib-4), or a stereoisomer thereof, or a pharmaceutically acceptable salt thereof disclosed herein can also be administered parenterally, in sterile liquid dosage forms, such as dispersions, suspensions or solutions. Other dosages forms that can also be used to administer the compound selected from Formulas (I), (Ia), (Ib), (Ib-1), (Ib-2), (Ib-3), (Ib-4), or a stereoisomer thereof, or a pharmaceutically acceptable salt thereof disclosed herein as an ointment, cream, drops, transdermal patch or powder for topical administration, as an ophthalmic solution or suspension formation, i.e., eye drops, for ocular administration, as an aerosol spray or powder composition for inhalation or intranasal administration, or as a cream, ointment, spray or suppository for rectal or vaginal administration.

Gelatin capsules containing the at least one compound and/or the at least one pharmaceutically acceptable salt thereof disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, suitable oil, saline, aqueous dextrose (glucose), and related sugar solutions and glycols such as propylene glycol or polyethylene glycols can be Examples of suitable carriers for parenteral solutions. Solutions for parenteral administration may comprise a water-soluble salt of the at least one compound describe herein, at least one suitable stabilizing agent, and if necessary, at least one buffer substance. Antioxidizing agents such as sodium bisulfate, sodium sulfite, or ascorbic acid, either alone or combined, can be Examples of suitable stabilizing agents. Citric acid and its salts and sodium EDTA can also be used as Examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol, a standard reference text in the art.

The compound selected from compounds of Formulas (I), (Ia), (I b), (Ib-1), (Ib-2), (Ib-3), (Ib-4), or a stereoisomer thereof, or a pharmaceutically acceptable salt thereof disclosed herein can further be examined for efficacy in treating cancer by in vivo assays. For example, the at least one compound and for the at least one pharmaceutically acceptable salts thereof disclosed herein can be administered to an animal (e.g., a mouse model) having cancer and its therapeutic effects can be accessed. Positive results in one or more of such tests are sufficient to increase the scientific storehouse of knowledge and hence sufficient to demonstrate practical utility of the compounds and for salts tested. Based on the results, an appropriate dosage range and administration route for animals, such as humans, can also be determined.

For administration by inhalation, the compound selected from compounds of Formulas (I), (Ia), (Ib), (Ib-1), (Ib-2), (Ib-3), (Ib-4), or a stereoisomer thereof, or a pharmaceutically acceptable salt thereof disclosed herein may be conveniently delivered in the form of an aerosol spray presentation from pressurized packs or nebulisers. The compound selected from compounds of Formulas (I), (Ia), (Ib), (Ib-1), (Ib-2), (Ib-3), (Ib-4), or a stereoisomer thereof, or a pharmaceutically acceptable salt thereof disclosed herein may also be delivered as powders, which may be formulated, and the powder composition may be inhaled with the aid of an insufflation powder inhaler device. One exemplary delivery system for inhalation can be a metered dose inhalation (MDI) aerosol, which may be formulated as a suspension or solution of a compound selected from compounds of Formulas (I), (Ia), (Ib), (Ib-1), (Ib-2), (Ib-3), (Ib-4), or a stereoisomer thereof, or a pharmaceutically acceptable salt thereof disclosed herein in at least one suitable propellant, selected, for example, from fluorocarbons and hydrocarbons.

For ocular administration, an ophthalmic preparation may be formulated with an appropriate weight percentage of a solution or suspension of the compound selected from compounds of Formulas (I), (Ia), (Ib), (Ib-1), (Ib-2), (Ib-3), (Ib-4), or a stereoisomer thereof, or a pharmaceutically acceptable salt thereof disclosed herein in an appropriate ophthalmic vehicle, such that the compound selected from compounds of Formulas (I), (Ia), (Ib), (Ib-1), (Ib-2), (Ib-3), (Ib-4), stereoisomers thereof, and at least one pharmaceutically acceptable salts thereof disclosed herein is maintained in contact with the ocular surface for a sufficient time period to allow the compound to penetrate the corneal and internal regions of the eye.

Useful pharmaceutical dosage-forms for administration of the compound selected from compounds of Formulas (I), (Ia), (Ib), (Ib-1), (Ib-2), (Ib-3), (Ib-4), or a stereoisomer thereof, or a pharmaceutically acceptable salt thereof disclosed herein include, but are not limited to, hard and soft gelatin capsules, tablets, parenteral injectables, and oral suspensions.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, a large number of unit capsules can be prepared by filling standard two-piece hard gelatin capsules each with, for example, 100 milligrams of the compound selected from compounds of Formulas (I), (Ia), (Ib), (Ib-1), (Ib-2), (Ib-3), (Ib-4), stereoisomers thereof, and pharmaceutically acceptable salt thereof disclosed herein in powder, 150 milligrams of lactose, 50 milligrams of cellulose, and 6 milligrams magnesium stearate.

In some embodiments, a mixture of the compound selected from compounds of Formulas (I), (Ia), (Ib), (Ib-1), (Ib-2), (Ib-3), (Ib-4), or a stereoisomer thereof, or a pharmaceutically acceptable salt thereof a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, a large number of tablets can be prepared by conventional procedures so that the dosage unit comprises, for example, 100 milligrams of the compound selected from compounds of Formulas (I), (Ia), (Ib), (Ib-1), (Ib-2), (Ib-3), (Ib-4), or a stereoisomer thereof, or a pharmaceutically acceptable salt thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the at least one compound and/or at least an enantiomer, a diastereomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided a compound selected from compounds of Formulas (I), (Ia), (Ib), (Ib-1), (Ib-2), (Ib-3), (Ib-4), or a stereoisomer thereof, or a pharmaceutically acceptable salt thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound selected from compounds of Formulas (I), (Ia), (Ib), (Ib-1), (Ib-2), (Ib-3), (Ib-4), or a stereoisomer thereof, or a pharmaceutically acceptable salt thereof are administered stepwise or in conjunction with at least one other therapeutic agent. When drugs are administered in physical combination, the dosage form and administration route should be selected depending on the compatibility of the combined drugs. Thus the term “coadministration” is understood to include the administration of at least two agents concomitantly or sequentially, or alternatively as a fixed dose combination of the at least two active components.

The compound selected from compounds of Formulas (I), (Ia), (Ib), (Ib-1), (Ib-2), (Ib-3), (Ib-4), stereoisomers thereof, and pharmaceutically acceptable salt thereof disclosed herein can be administered as the sole active ingredient or in combination with at least one second active ingredient, selected, for example, from other active ingredients known to be useful for treating cancers in a patient.

### EXAMPLES

The Examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless otherwise indicated.

Unless otherwise indicated, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and for heat dried.

Unless otherwise indicated, column chromatography purification was conducted on a Biotage system (Manufacturer: Dyax Corporation) having a silica gel column or on a silica SepPak cartridge (Waters), or was conducted on a Teledyne Isco Combiflash purification system using prepacked silica gel cartridges.

¹H NMR spectra were recorded on a Varian instrument operating at 400 MHz. ¹H-NMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₂CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz). All compound names except the reagents were generated by ChemDraw version 12.0.

In the following Examples, the abbreviations below are used:
AcOH Acetic acid
Aq Aqueous
Brine Saturated aqueous sodium chloride solution
Bn Benzyl
BnBr Benzyl Bromide
Boc Tert-butyloxycarbonyl
Cbz benzyloxycarbonyl
CH₂Cl₂ Dichloromethane
DMF N,N-Dimethylformamide
Dppf 1,1″-bis(diphenylphosphino)ferrocene
DBU 1,8-diazabicyclo[5.4.0]undec-7-ene
DCM Dichloromethane
DIEA or DIPEA N,N-diisopropylethylamine
DIBAL-H Diisobutylaluminium hydride
DMAP 4-N,N-dimethylaminopyridine
DMF N,N-dimethylformamide
DMSO Dimethyl sulfoxide
EA or EtOAc Ethyl acetate
EtOH Ethanol
Et₂O or ether Diethyl ether
g Grams
h or hr Hour
HATU O-(7-Azabenzotriazol-1-yl)-N,N,N′,N′-tetramethyluronium hexafluorophosphate
HBTU O-(7-Azabenzotriazol-1-yl)-N,N,N′,N′-tetramethyluronium hexafluorophosphate
HCl Hydrochloric acid
Hex Hexane
HPLC High-performance liquid chromatography
IPA 2-propanol
i-PrOH Isopropyl alcohol
mg Milligrams
mL Milliliters
Mmol Millimole
MeCN Acetonitrile
MeOH Methanol
Min Minutes
ms or MS Mass spectrum
Na₂SO₄ Sodium sulfate
PE Petroleum ether
Ph₃PO Triphenyl phosphorus oxide
PPA Polyphosphoric acid
Rt Retention time
Rt or rt Room temperature
TBAF Tetra-butyl ammonium fluoride
TBSCI tert-Butyldimethylsilyl chloride
TEA Triethanolamine
TFA Trifluoroacetic acid
Tf₂O Triflic anhydride
THF Tetrahydrofuran
TLC thin layer chromatography
Ts para-toluenesulfonyl
TBS tert-butyldimethylsilyl
μL Microliters

### Synthesis of Substituted Benzo[d]Imidazols

### Example 1: 2-(1-((1s,4s)-4-(6-fluoroquinolin-4-yl)cyclohexyl)ethyl)-N-methyl-1H-benzo[d]imidazole-5-carboxamide

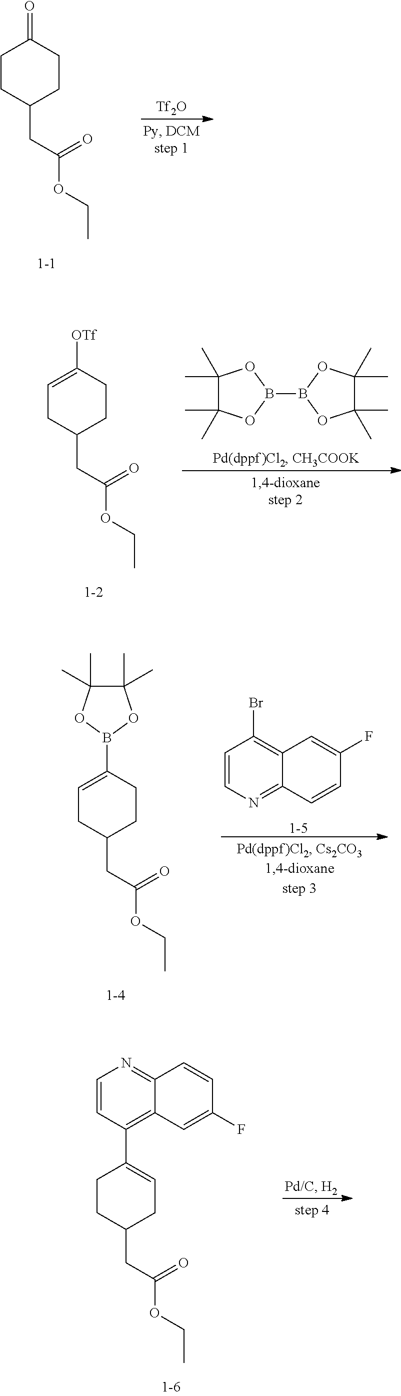

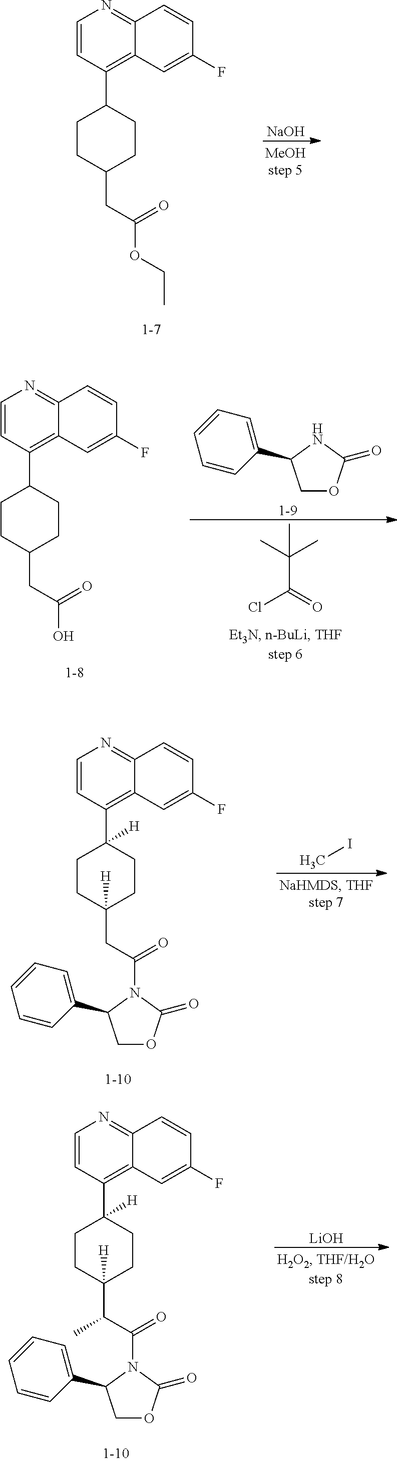

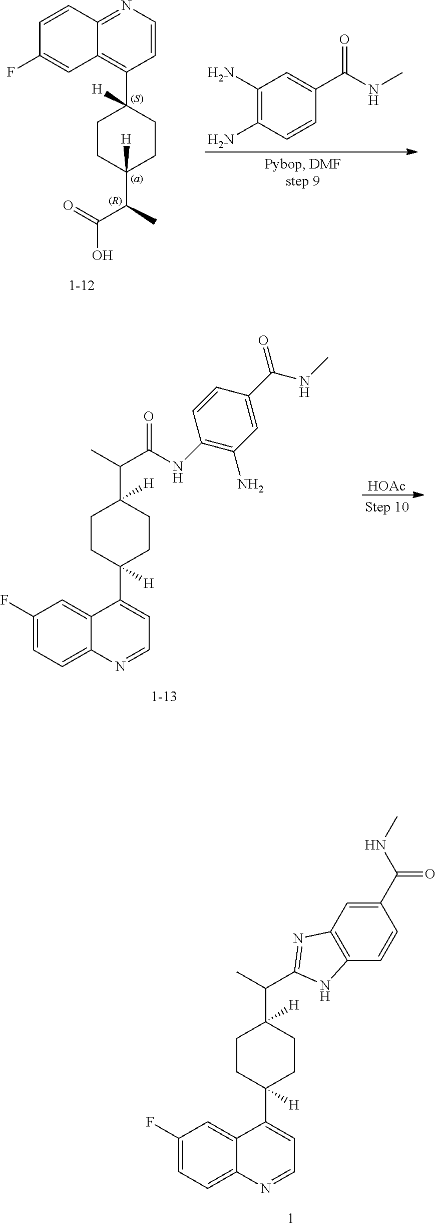

### Step 1: ethyl 2-(4-(((trifluoromethyl)sulfonyl)oxy)cyclohex-3-en-1-yl)acetate

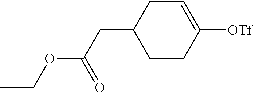 [SMILES: CCOC(=O)CC1CC=C(OS(=O)(=O)C(F)(F)F)CC1]

To a solution of ethyl 2-(4-oxocyclohexyl)acetate (18.4 g, 100 mmol, 1.00 eq) dissolved in DCM (250 ml) were added pyridine (9.48 g, 120 mmol, 1.20 eq) and Tf₂O (42.15 g, 150 mmol, 1.50 eq). The mixture was stirred at room temperature overnight. The solution was washed with water (400 ml), saturated ammonium chloride (400 ml) and brine (400 ml). The organic layers were combined, dried over Na₂SO₄, filtered and concentrated to dryness. The crude (30.32 g, 95% yield) was used for next step without further purification. ¹H NMR (CDCl₃) δ_{H} 5.72 (s, 1H), 4.15 (q, J=7.2 Hz, 2H), 2.39-2.51 (m, 1H), 2.28-2.38 (m, 4H), 2.08-2.21 (m, 1H), 1.87-1.98 (m, 2H), 1.45-1.57 (m, 1H) and 1.27 (t, J=7.2 Hz, 3H).

### Step 2: ethyl 2-(4-(4,4,5,5-tetramethyl-1,3,2-dioxaborolan-2-yl)cyclohex-3-en-1-yl)acetate

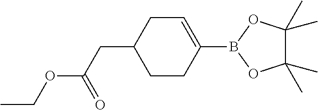 [SMILES: CCOC(=O)CC1CC=C(B2OC(C)(C)C(C)(C)O2)CC1]

To a mixture of ethyl 2-(4-(((trifluoromethyl)sulfonyl)oxy)cyclohex-3-en-1-yl)acetate (30.32 g, crude, 96 mmol, 1.00 eq) dissolved in 1,4-dioxane (400 ml), 4,4,4′,4′,5,5,5′,5′-octamethyl-2,2′-bi(1,3,2-dioxaborolane) (26.8 g, 106 mmol, 1.10 eq), CH3COOK (38.02 g, 192 mmol, 2.00 eq) and Pd(dppf)Cl₂ (14.04 g, 19.2 mmol, 0.20 eq) were added. The mixture was stirred at 95° C. under nitrogen protection for 18 hours. The solution was filtered and concentrated to dryness. The crude (12.50 g, 100% yield) was filtered through the silica gel pad and washed with PE/EA=6:1. The filtrate was concentrated to dryness to give a black oil (33.2 g, 112.3% yield) which was used in next step without further purification. ¹H NMR (CDCl₃) δ_{H} 6.51 (s, 1H), 4.13 (q, J=7.2 Hz, 3H), 1.99-2.40 (m, 9H), 1.68-1.94 (m, 3H) and 1.18-1.26 (m, 12H).

### Step 3: ethyl 2-(4-(6-fluoroquinolin-4-yl)cyclohex-3-en-1-yl)acetate

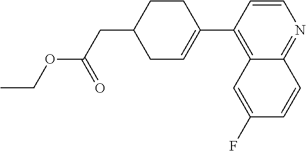 [SMILES: CCOC(=O)CC1CC=C(c2ccnc3ccc(F)cc23)CC1]

Ethyl 2-(4-(4,4,5,5-tetramethyl-1,3,2-dioxaborolan-2-yl)cyclohex-3-en-1-yl)acetate (33.2 g, 110 mmol, 1.10 eq) was dissolved in 1,4-dioxane (450 ml) and was added with 4-bromo-6-fluoroquinoline (22.5 g 100 mmol, 1.00 eq), Cs₂CO₃ (65 g, 200 mmol, 2.00 eq) and Pd(dppf)Cl₂ (14.62 g, 20 mmol, 0.20 eq). The mixture was stirred at 95° C. under nitrogen protection for 18 hours. The solution was filtered and concentrated to dryness. The crude was purified by column chromatography on silica gel 200 g (PE/EA=10/1 to 4/1) to give a clear oil (12.02 g, 34.8 yield). ¹H NMR (CDCl₃) δ_{H} 8.80 (d, J=4.4 Hz, 1H), 8.15 (dd, J=9.2, 5.6 Hz, 1H), 7.62 (dd, J=10.0, 2.8 Hz, 1H), 7.49 (m, 1H), 7.21 (d, J=4.4 Hz, 1H), 5.81-5.87 (m, 1H), 4.19 (q, J=7.2 Hz, 2H), 2.23-2.57 (m, 6H), 1.95-2.04 (m, 2H), 1.53-1.65 (m, 1H) and 1.30 (t, J=7.2 Hz, 3H).

### Step 4: ethyl 2-(4-(6-fluoroquinolin-4-yl)cyclohexyl)acetate

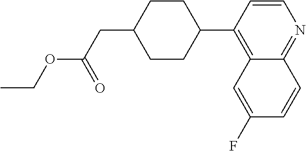 [SMILES: CCOC(=O)CC1CCC(c2ccnc3ccc(F)cc23)CC1]

To a mixture of ethyl 2-(4-(6-fluoroquinolin-4-yl)cyclohex-3-en-1-yl)acetate (12.02 g, 38 mmol, 1.00 eq) dissolved in MeOH (50 ml) was added Pd/C (2.4 g, w.t. 20%), and the mixture was stirred at room temperature under a hydrogen balloon overnight. Then the mixture was filtered and concentrated to dryness. The crude was purified by column chromatography on silica gel 150 g (PE/EA=10/1 to 2/1) to give a pale yellow oil (8.51 g, 70.3% yield). ¹H NMR (CDCl₃) δ_{H} 8.77-8.86 (m, 1H), 8.15 (dd, J=9.2, 5.6 Hz, 1H), 7.66 (dd, J=10.4, 2.4 Hz, 1H), 7.44-7.52 (m, 1H), 7.28-7.38 (m, 1H), 4.16 (q, J=7.2 Hz, 2H), 3.07-3.32 (m, 1H), 2.45-2.53 (m, 2H), 1.92-2.10 (m, 3H), 1.53-1.89 (m, 6H) and 1.28 (t, J=7.2, 4.0 Hz, 3H).

### Step 5: 2-(4-(6-fluoroquinolin-4-yl)cyclohexyl)acetic Acid

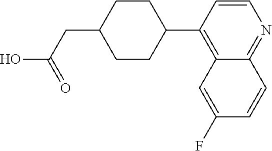 [SMILES: O=C(O)CC1CCC(c2ccnc3ccc(F)cc23)CC1]

To a mixture of ethyl 2-(4-(6-fluoroquinolin-4-yl)cyclohexyl)acetate (8.51 g, 27 mmol, 1.00 eq) dissolved in MeOH (20 ml) and water (20 ml) was added NaOH (1.61 g, 40.5 mmol, 1.50 eq). The mixture was stirred at room temperature for 2 hours. The solution was concentrated to 20 ml and extracted with EA (20 mL×3) to remove the impurities. The water layer was concentrated to 5 ml. The water layer was neutralized with 1N HCl to make the PH to 7. Then the mixture was added to water (200 ml) and extracted with DCM/MeOH (20/1, 400 ml×3). The organic layers were dried over Na₂SO₄, filtered and concentrated to give the crude product, which was recrystallized in water to give the product (7.74 g). ¹H NMR (DMSO-d₆) δ_{H} 8.84 (t, J=4.4 Hz, 1H), 8.11-8.25 (m, 1H), 7.66 (dd, J=10.4, 2.4 Hz, 1H), 7.44-7.54 (m, 1H), 7.28-7.40 (m, 1H), 3.09-3.32 (m, 1H), 2.31-2.64 (m, 3H), 1.96-2.10 (m, 2H), 1.72-1.91 (m, 4H), 1.56-1.69 (m, 1H) and 1.29-1.45 (m, 1H).

Step 6: (R)-3-(2-((1s,4S)-4-(6-fluoroquinolin-4-yl)cyclohexyl)acetyl)-4-phenyloxazolidin-2-one

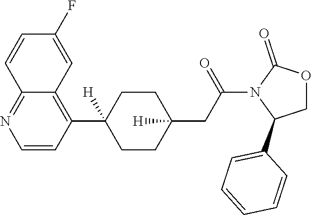

To a flask #a were added 2-(4-(6-fluoroquinolin-4-yl)cyclohexyl)acetic acid (7.74 g, 27 mmol, 1.00 eq), THF (250 ml) and TEA (8.5 ml, 2.00 eq). The mixture was stirred at −78° C. for 0.5 hours. Pivaloyl chloride (3.5 ml, 1.95 eq) was added to the flask dropwised under nitrogen protection. Then the mixture was warmed to 0° C. and stirred for 1 hour.

To a flask #b were added (R)-4-phenyloxazolidin-2-one (3.55 g, 29 mmol, 1.10 eq) and THF (60 ml). The solution was cooled to −78° C. before the careful addition of n-BuLi (1.6 N, 34 ml, 2.00 eq). And the mixture was stirred at −78° C. for 0.5 hour.

Flask #a was then cooled to −78° C. and the contents of Flask #b were added to Flask #a via a cannula over the course of 15 minutes. After addition was completed, the cold bath was removed, and the mixture was stirred for 3 hours at room temperature. The reaction mixture was quenched with saturated ammonium chloride solution (500 ml) and extracted with EA (500 ml×3). The organic layers were combined, dried over Na₂SO₄, filtered and concentrated to dryness. The crude was purified by column chromatography on silica gel 100 g (PE/EA=4/1 to 1/1) to give a product as a white solid, which was slurried in 2-methoxy-2-methylpropane to give the product as cis-product, and the mother liquid was cis and trans mixture. Cis-product ¹H NMR (CDCl₃) δ_{H} 8.77-8.86 (m, 1H), 8.24 (s, 1H), 7.66 (dd, J=10.2, 2.4 Hz, 1H), 7.51 (t, J=8.4 Hz, 1H), 7.28-7.45 (m, 6H), 5.47 (dd, J=8.8, 3.6 Hz, 1H), 4.68-4.79 (m, 2H), 4.26-4.35 (m, 1H), 2.93-3.27 (m, 2H), 2.41-2.56 (m, 1H), 1.89-2.01 (m, 2H), 1.67-1.84 (m, 4H), 1.47-1.63 (m, 1H), 1.28-1.39 (m, 1H).

### Step 7: (R)-3-((R)-2-((1s,4S)-4-(6-fluoroquinolin-4-yl)cyclohexyl)propanoyl)-4-phenyloxazolidin-2-one

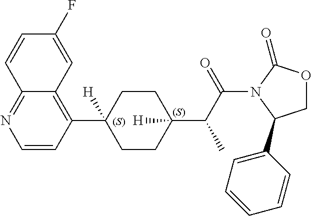

To a solution of NaHMDS (1.0 N, 14 ml, 2.00 eq) was added (R)-3-(2-((1s,4S)-4-(6-fluoroquinolin-4-yl)cyclohexyl)acetyl)-4-phenyloxazolidin-2-one (3.55 g, 8 mmol, 1.00 eq) in THF (80 ml) at −78° C. The mixture was warmed to −20° C. and stirred for 1 hour. Then the mixture was cooled to −78° C. and added iodomethane (7.50 g, 3.3 ml, 5.00 eq). The mixture was stirred at this temperature for 2 hours and quenched with saturated ammonium chloride solution (100 ml) and extracted with EA (100 ml×3). The organic layers were combined, dried over Na₂SO₄, filtered and concentrated to dryness. The crude was purified by column chromatography on silica gel 100 g (PE/EA=4/1 to 1/1) to give a product (2.11 g, 41% yield) as a pale yellow solid. ¹H NMR (CDCl₃) δ_{H} 8.77-8.85 (m, 1H), 8.09-8.18 (m, 1H), 7.62-7.70 (m, 1H), 7.44-7.50 (m, 1H), 7.29-7.44 (m, 6H), 5.38-5.52 (m, 2H), 4.91-5.00 (m, 1H), 4.66-4.79 (m, 2H), 4.16-4.38 (m, 2H), 2.10-2.20 (m, 1H), 1.86-2.03 (m, 2H), 1.65-1.83 (m, 4H), 1.45-1.64 (m, 1H), 1.12 (t, J=7.2 Hz, 2H).

### Step 8: (R)-2-((1s,4S)-4-(6-fluoroquinolin-4-yl)cyclohexyl)propanoic Acid

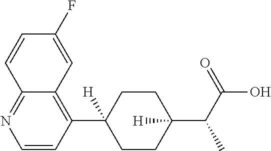

To a solution of (R)-3-((R)-2-((1s,4S)-4-(6-fluoroquinolin-4-yl)cyclohexyl)propanoyl)-4-phenyloxazolidin-2-one (2.71 g, 6 mmol, 1.00 eq) dissolved in THF (40 ml) and water (10 ml) was added H₂O₂ (5 ml) dropwised at 0° C. The mixture was stirred at 0° C. for 1 hour. Then the mixture was added with LiOH (2 N, 6 ml, 2.00 eq) and stirred at room temperature for 4 hours. Progress was followed by LC/MS and the mixture was carefully quenched at 0° C. by the addition of saturated Na₂SO₃, once starting material had been consumed. The PH was adjusted to 5˜6 with 1N HCl and then the mixture was extracted with DCM/MeOH (40/1, 50 ml×4). The organic layers were dried over Na₂SO₄, filtered and concentrated to dryness to get a pale yellow solid (1.02 g, 55% yield). ¹H NMR (CDCl₃) δ_{H} 8.82 (d, J=4.6 Hz, 1H), 8.11-8.20 (m, 1H), 7.63-7.72 (m, 1H), 7.45-7.53 (m, 1H), 7.27-7.33 (m, 1H), 3.12-3.33 (m, 1H), 2.34-2.49 (m, 1H), 1.57-2.14 (m, 9H), 1.20-1.29 (m, 3H).

### Step 9: 3-amino-4-(2-((1s,4s)-4-(6-fluoroquinolin-4-yl)cyclohexyl)propanamido)-N-methylbenzamide

A solution of (R)-2-((1s,4s)-4-(6-fluoroquinolin-4-yl)cyclohexyl)propanoic acid (100 mg, 0.33 mmol) in DMF (10 mL) were added Pybop (250 mg, 0.48 mmol), DIPEA (200 mg, 1.55 mmol) and N⁴-methylbenzene-1,2,4-triamine (100 mg, 0.73 mmol) was heated to 60° C. for 4 hours. After cooled down, EA was added, washed with water and brine, dried over Na₂SO₄, concentrated and purified by sili-gel (DCM:MeOH=5:100-10:100) to give the compound 1-13 (20 mg).

### Step 10: 2-(1-((1s,4s)-4-(6-fluoroquinolin-4-yl)cyclohexyl)ethyl)-N-methyl-1H-benzo[d]imidazole-5-carboxamide

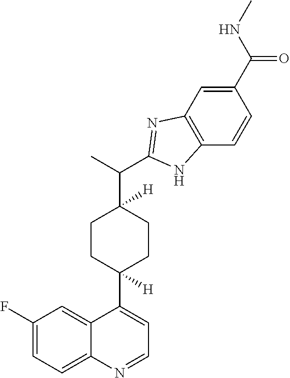 [SMILES: CNC(=O)c1ccc2[nH]c(C(C)[C@H]3CC[C@@H](c4ccnc5ccc(F)cc54)CC3)nc2c1]

A solution of compound 1-13 (20 mg) in HOAc (4 mL) was stirred at 110° C. for 16 hours, then the solvent was evaporated. The crude residue was dissolved with EA (50 mL) and washed with saturated NaHCO₃ solution (50 mL). Separated the organic phase and purified by pre-HPLC to give the title compound. ¹H NMR (400 MHz, DMSO-d) δ_{H} 8.79 (d, J=4.7 Hz, 1H), 8.09-8.06 (m, 2H), 7.89-7.86 (m, 1H), 7.77-7.46 (m, 4H), 3.56-3.39 (m, 2H), 2.94 (s, 3H), 2.28-2.14 (m, 2H), 2.00-1.90 (m, 4H), 1.78-1.69 (m, 2H), 1.47-1.45 (m, 3H), 1.31 (d, J=12.1 Hz, 1H).

Example 2 (Compound 2) was prepared in a procedure similar to Example 1.

### Example 2: 2-((R)-1-((1s,4S)-4-(6-fluoroquinolin-4-yl)cyclohexyl)ethyl)-N-isopropyl-1H-benzo[d]imidazole-6-carboxamide

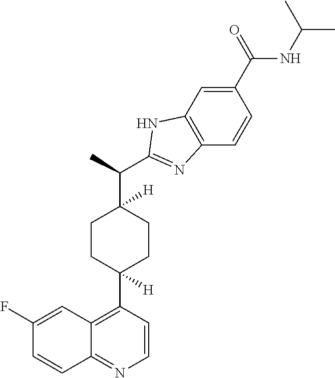 [SMILES: CC(C)NC(=O)c1ccc2nc([C@H](C)[C@H]3CC[C@@H](c4ccnc5ccc(F)cc54)CC3)[nH]c2c1]

¹H NMR (400 MHz, DMSO-d) δ_{H} 12.40 (d, J=10.6 Hz, 1H), 8.87 (d, J=4.4 Hz, 1H), 8.15-8.08 (m, 3H), 7.98 (dd, J=11.0, 2.7 Hz, 1H), 7.92 (s, 0.5H), 7.71-7.62 (m, 2H), 7.58 (d, J=4.5 Hz, 1H), 7.55-7.43 (m, 1H), 4.14-4.09 (m, 1H), 3.42-3.42 (m, 2H), 2.20-2.00 (m, 2H), 1.95-1.70 (m, 4H), 1.69-1.50 (m, 2H), 1.36 (d, J=6.7 Hz, 3H), 1.17 (d, J=6.5 Hz, 6H).

### Example 3: N-(tert-butyl)-2-((R)-1-((1s,4S)-4-(6-fluoroquinolin-4-yl)cyclohexyl)ethyl)-1H-benzo[d]imidazole-6-carboxamide

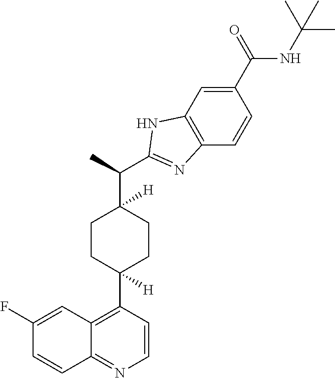 [SMILES: C[C@@H](c1nc2ccc(C(=O)NC(C)(C)C)cc2[nH]1)[C@H]1CC[C@@H](c2ccnc3ccc(F)cc32)CC1]

¹H NMR (400 MHz, DMSO-d) δ_{H} 12.39 (s, 1H), 8.87 (d, J=4.5 Hz, 1H), 8.14-7.82 (m, 3H), 7.72-7.37 (m, 5H), 3.45-3.40 (m, 2H), 2.19-1.96 (m, 2H), 1.95-1.72 (m, 4H), 1.67-1.54 (m, 2H), 1.43-1.34 (m, 12H), 1.16-1.13 (m, 1H).

### Example 4: N-cyclobutyl-2-((R)-1-((1s,4S)-4-(6-fluoroquinolin-4-yl)cyclohexyl)ethyl)-1H-benzo[d]imidazole-6-carboxamide

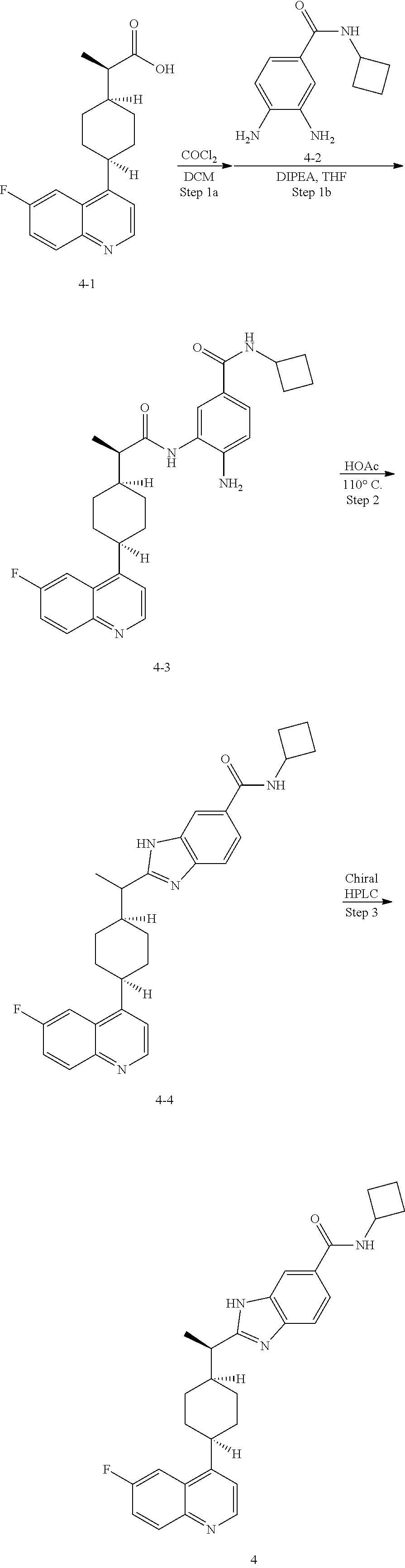

### Step 1a and 1b: 4-amino-N-cyclobutyl-3-(2(1s,4s)-4-(6-fluoroquinolin-4-yl)cyclohexyl)propanamido)benzamide

To a solution of (R)-2-((1s,4s)-4-(6-fluoroquinolin-4-yl)cyclohexyl)propanoic acid (400 mg, 1.33 mmol) in DCM (10 mL) were added DMF (1 drop) and oxalyl chloride (200 mg, 1.55 mmol) dropwise, the mixture was stirred for 2 hours at room temperature, concentrated to give the crude 2-((1s,4s)-4-(6-fluoroquinolin-4-yl)cyclohexyl)propanoyl chloride. At 0° C., to a solution of 3,4-diamino-N-cyclobutylbenzamide and DIPEA in THF was added a solution of 2-((1s,4s)-4-(6-fluoroquinolin-4-yl)cyclohexyl)propanoyl chloride in THF dropwise, the reaction mixture was stirred for 30 min at 0° C., the reaction mixture was concentrated, EA was added, washed with water and brine, dried over Na₂SO₄, concentrated and purified by sili-gel to give 4-amino-N-cyclobutyl-3-(2-((1s,4s)-4-(6-fluoroquinolin-4-yl)cyclohexyl)propanamido)benzamide.

### Step 2: N-cyclobutyl-2-(1-((1s,4s)-4-(6-fluoroquinolin-4-yl)cyclohexyl)ethyl)-1H-benzo[d]imidazole-6-carboxamide

A solution of 4-amino-N-cyclobutyl-3-(2-((1s,4s)-4-(6-fluoroquinolin-4-yl)cyclohexyl)propanamido)benzamide in HOAc was heated to 110° C. for 8 hours, after cooled down, the mixture was concentrated, saturated NaHCO₃.aq was added, extracted with EA, the EA layer was washed with brine, dried over Na₂SO₄, concentrated and purified by sili-gel to give N-cyclobutyl-2-(1-((1s,4s)-4-(6-fluoroquinolin-4-yl)cyclohexyl)ethyl)-1H-benzo[d]imidazole-6-carboxamide. ¹H NMR (400 MHz, DMSO-d) δ_{H} 12.43 (s, 1H), 8.87 (d, J=4.5 Hz, 1H), 8.52 (s, 1H), 8.12-8.08 (m, 1H), 7.98 (dd, J=11.0, 2.6 Hz, 1H), 7.69-7.64 (m, 2H), 7.59-7.47 (m, 2H), 4.44 (dd, J=16.3, 8.2 Hz, 1H), 3.45-3.41 (m, Hz, 2H), 2.28-1.98 (m, 6H), 1.95-1.73 (m, 4H), 1.72-1.52 (m, 4H), 1.36 (d, J=6.8 Hz, 3H), 1.17 (d, J=12.4 Hz, 1H).

### Step 3: N-cyclohexyl-2-((R)-1-((1s,4S)-4-(6-fluoroquinolin-4-yl)cyclohexyl)ethyl)-1H-benzo[d]imidazole-6-carboxamide

53 mg compound example 4 was separated from 65 mg of compound 4-4 by using preparative HPLC on a CHIRALPAK IC with Hex(8 mM NH3-MeOH):EtOH=70:30 as an eluent, the desired enantiomer eluted at the retention time of 2.483 min. ¹H NMR (MeOH-d₆) δ_{H} 12.46 (s, 1H), 8.87 (d, J=4.5 Hz, 1H), 8.53 (s, 1H), 8.10 (dd, J=9.2, 5.8 Hz, 1H), 8.02-7.93 (m, 1H), 7.69-7.64 (1n, 2H), 7.59 (d, J=4.3 Hz, 1H), 7.50 (s, 1H), 4.44 (dd, J=16.0, 7.9 Hz, 1H), 3.45-3.39 (m, 2H), 2.28-2.00 (m, 7H), 1.96-1.74 (m, 4H), 1.71-1.50 (m, 4H), 1.36 (d, J=6.8 Hz, 3H), 1.16 (d, J=11.7 Hz, 1H).

### Example 5: N-cyclopentyl-2-((R)-1-((1s,4S)-4-(6-fluoroquinolin-4-yl)cyclohexyl)ethyl)-1H-benzo[d]imidazole-6-carboxamide

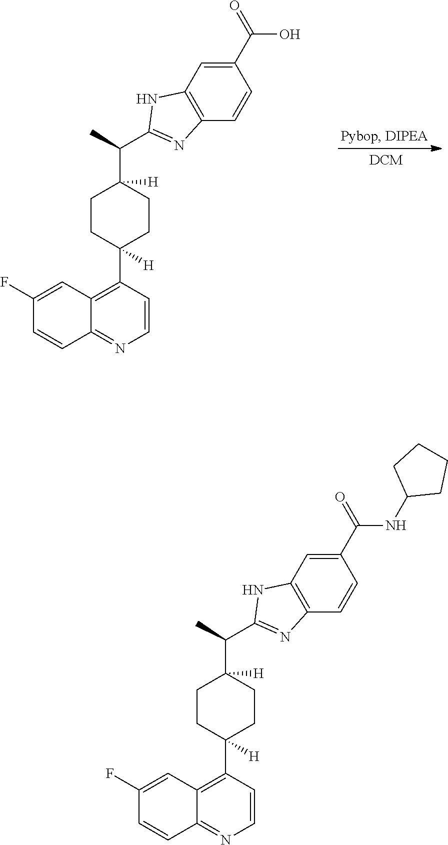 [SMILES: C[C@@H](c1nc2ccc(C(=O)O)cc2[nH]1)[C@H]1CC[C@@H](c2ccnc3ccc(F)cc32)CC1 ; C[C@@H](c1nc2ccc(C(=O)NC3CCCC3)cc2[nH]1)[C@H]1CC[C@@H](c2ccnc3ccc(F)cc32)CC1]

A solution of 2-((R)-1-((1s,4S)-4-(6-fluoroquinolin-4-yl)cyclohexyl)ethyl)-1H-benzo[d]imidazole-6-carboxylic acid (90 mg, 0.21 mmol), cyclopentanamine (35 mg, 0.4 mmol), PyBop (160 mg, 0.31 mmol) and Et₃N (130 mg, 1.3 mmol) in DCM (10 mL) was stirred for 16 hours at room temperature, the reaction mixture was concentrated, EA was added, washed with water and brine, dried over Na₂SO₄, concentrated and purified by sili-gel to give N-cyclopentyl-2-((R)-1-((1s,4S)-4-(6-fluoroquinolin-4-yl)cyclohexyl)ethyl)-1H-benzo[d]imidazole-6-carboxamide. ¹H NMR (400 MHz, DMSO-d) Sir 12.42 (s, 1H), 8.87 (d, J=4.5 Hz, 1H), 8.19 (s, 1H), 8.10 (dd, J=9.2, 5.8 Hz, 1H), 7.98 (dd, J=11.0, 2.7 Hz, 1H), 7.68-7.64 (m, 2H), 7.58 (d, J=4.6 Hz, 1H), 7.50-7.48 (m, 1H), 4.24 (dd, J=13.6, 6.9 Hz, 1H), 3.45-3.39 (m, 2H), 3.01 (td, J=6.6, 4.0 Hz, 1H), 2.16-2.03 (m, 2H), 1.91-1.81 (m, 4H), 1.76-1.67 (m, 5H), 1.61-1.53 (m, 4H), 1.36 (d, J=6.8 Hz, 3H), 1.20-1.12 (m, 1H).

Example 6 (Compound 6) was prepared in a procedure similar to Example 5

### Example 6: N-cyclohexyl-2-((R)-1-((1s,4S)-4-(6-fluoroquinolin-4-yl)cyclohexyl)ethyl)-1H-benzo[d]imidazole-6-carboxamide

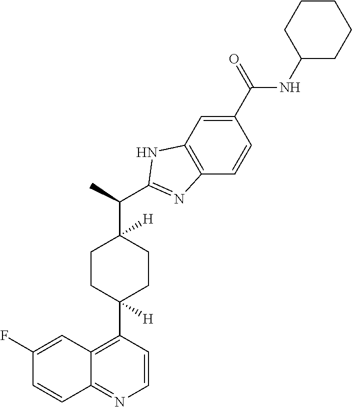 [SMILES: C[C@@H](c1nc2ccc(C(=O)NC3CCCCC3)cc2[nH]1)[C@H]1CC[C@@H](c2ccnc3ccc(F)cc32)CC1]

¹H NMR (400 MHz, DMSO-d) δ_{H} 12.43 (s, 1H), 8.87 (d, J=4.5 Hz, 1H), 8.10 (dd, J=9.2, 5.9 Hz, 2H), 7.98 (dd, J=11.0, 2.7 Hz, 1H), 7.71-7.61 (m, 2H), 7.58 (d, J=4.6 Hz, 1H), 7.49-7.47 (m, 1H), 3.77 (s, 1H), 3.45-3.41 (m, 2H), 2.19-2.00 (m, 2H), 1.97-1.76 (m, 6H), 1.72-1.70 (m, 3H), 1.66-1.51 (m, 3H), 1.36 (d, J=6.9 Hz, 3H), 1.31-1.28 (m, 3H), 1.17-1.14 (m, 2H).

### Example 7: 2-(1-((1s,4s)-4-(6-fluoroquinolin-4-yl)cyclohexyl)ethyl)-N-methoxy-1H-benzo[d]imidazole-6-carboxamide

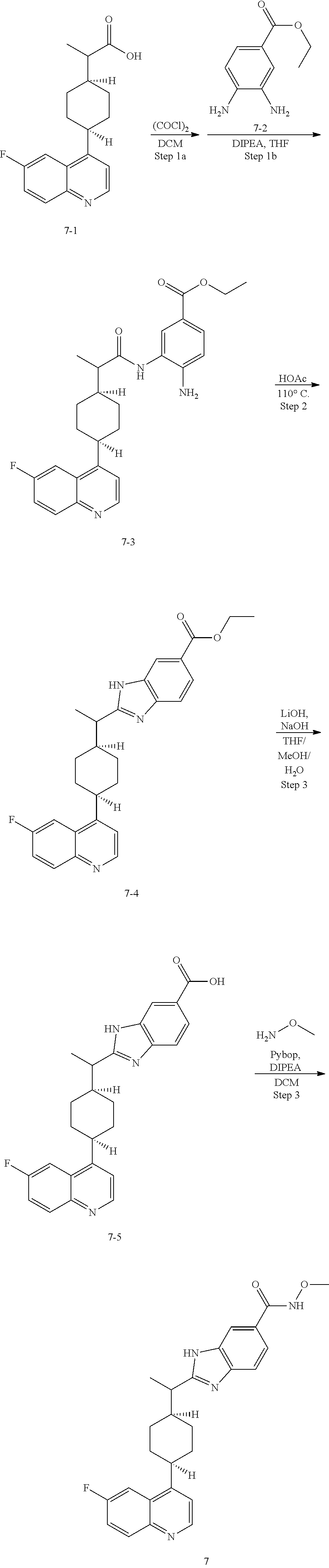

### Step 1a and 1b: Ethyl 4-amino-3-(2-((1s,4s)-4-(6-fluoroquinolin-4-yl)cyclohexyl)-propanamido)benzoate

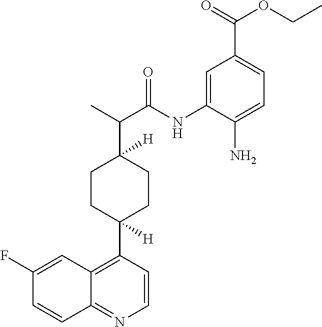 [SMILES: CCOC(=O)c1ccc(N)c(NC(=O)C(C)[C@H]2CC[C@@H](c3ccnc4ccc(F)cc43)CC2)c1]

To a solution of 2-((1s,4s)-4-(6-fluoroquinolin-4-yl)cyclohexyl)propanoic acid (4.0 g, 13.3 mmol) in DCM (100 mL) were added DMF (1 drop) and oxalyl chloride (2.5 mL, 30 mmol) dropwise, the mixture was stirred for 2 hours at room temperature, concentrated to give the crude 2-((1s,4s)-4-(6-fluoroquinolin-4-yl)cyclohexyl)propanoyl chloride (step 1a). At 0° C., to the solution of ethyl 3,4-diaminobenzoate (2.5 g, 14 mmol) and DIPEA (3.8 g, 29 mmol) in THF (150 mL) was added a solution of 2-((1s,4s)-4-(6-fluoroquinolin-4-yl)cyclohexyl)propanoyl chloride in THF (50 mL) dropwise, the reaction mixture was stirred for 30 min at 0° C., the reaction mixture was concentrated, EA was added, washed with water and brine, dried over Na₂SO₄, concentrated and purified by sili-gel to give 4.3 g ethyl 4-amino-3-(2-((1s,4s)-4-(6-fluoroquinolin-4-yl)cyclohexyl)propanamido)benzoate.

### Step 2: Ethyl 2-(1-((1s,4s)-4-(6-fluoroquinolin-4-yl)cyclohexyl)ethyl)-1H-benzo[d]imidazole-6-carboxylate

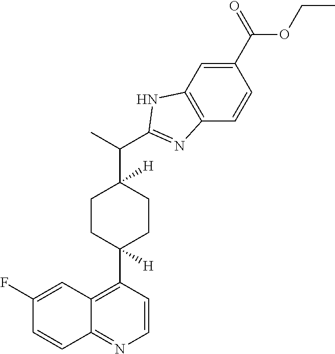 [SMILES: CCOC(=O)c1ccc2nc(C(C)[C@H]3CC[C@@H](c4ccnc5ccc(F)cc54)CC3)[nH]c2c1]

A solution of ethyl 4-amino-3-(2-((1s,4s)-4-(6-fluoroquinolin-4-yl)cyclohexyl)propanamido)benzoate (4.3 g) in HOAc (150 mL) was heated to 110° C. for 8 hours, after cooled down, the mixture was concentrated, saturated NaHCO₃.aq was added, extracted with EA, the EA layer was washed with brine, dried over Na₂SO₄, concentrated and purified by sili-gel to give ethyl 2-(1-((1s,4s)-4-(6-fluoroquinolin-4-yl)cyclohexyl)ethyl)-1H-benzo[d]imidazole-6-carboxylate. ¹H NMR (400 MHz, DMSO-d) δ_{H} 8.87 (d, J=4.5 Hz, 1H), 8.13-8.08 (m, 2H), 7.99 (dd, J=10.9, 2.5 Hz, 1H), 7.82 (dd, J=8.5, 1.4 Hz, 1H), 7.67 (td, J=8.7, 2.6 Hz, 1H), 7.63-7.55 (m, 2H), 4.32 (q, J=7.1 Hz, 2H), 3.55-3.42 (m, 2H), 2.18-2.15 (m, 1H), 2.11-2.01 (m, 1H), 1.95-1.73 (m, 4H), 1.71-1.52 (m, 2H), 1.38 (d, J=6.8 Hz, 3H), 1.34 (t, J=7.1 Hz, 3H), 1.18-1.14 (m, 1H).

### Step 3: 2-(1-((1s,4s)-4-(6-fluoroquinolin-4-yl)cyclohexyl)ethyl)-1H-benzo[d]-imidazole-6-carboxylic Acid

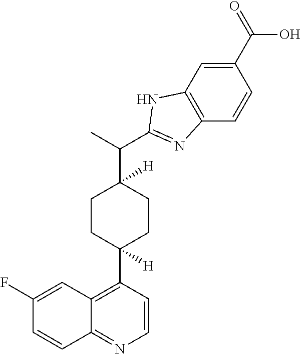 [SMILES: CC(c1nc2ccc(C(=O)O)cc2[nH]1)[C@H]1CC[C@@H](c2ccnc3ccc(F)cc32)CC1]

A solution of ethyl 2-(1-((1s,4s)-4-(6-fluoroquinolin-4-yl)cyclohexyl)ethyl)-1H-benzo[d]imidazole-6-carboxylate (200 mg, 0.45 mmol) and LiOH·H₂O (260 mg, 6.2 mmol), NaOH (200 mg, 5.0 mmol) in THE/MeOH/H₂O (10 mL/10 mL/10 mL) was stirred for 48 hours at room temperature, the reaction mixture was concentrated, the residue's pH value was adjusted to 6 with 1N HCl.aq, the white solid was collected and dried in vacuo to give 160 mg crude 2-(1-((1s,4s)-4-(6-fluoroquinolin-4-yl)cyclohexyl)ethyl)-1H-benzo[d]imidazole-6-carboxylic acid.

### Step 4: 2-(1-((1s,4s)-4-(6-fluoroquinolin-4-yl)cyclohexyl)ethyl)-N-methoxy-1H-benzo[d]imidazole-6-carboxamide

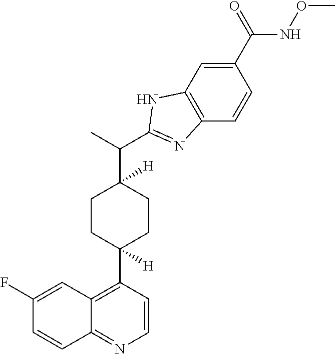 [SMILES: CONC(=O)c1ccc2nc(C(C)[C@H]3CC[C@@H](c4ccnc5ccc(F)cc54)CC3)[nH]c2c1]

A solution of 2-(1-((1s,4s)-4-(6-fluoroquinolin-4-yl)cyclohexyl)ethyl)-1H-benzo[d]imidazole-6-carboxylic acid (130 mg, 0.31 mmol), methoxylamine hydrochloride (50 mg, 0.6 mmol), PyBop (280 mg, 0.54 mmol) and DIPEA (0.5 mL, 2.9 mmol) in DCM (15 mL) was stirred for 16 hours at room temperature, the reaction mixture was concentrated, EA was added, washed with water and brine, dried over Na₂SO₄, concentrated and purified by sili-gel to give 35 mg 2-(1-((1s,4s)-4-(6-fluoroquinolin-4-yl)cyclohexyl)ethyl)-N-methoxy-1H-benzo[d]imidazole-6-carboxamide. ¹H NMR (400 MHz, DMSO-d) δ_{H} 12.52 (s, 1H), 11.64 (s, 1H), 8.87 (d, J=4.4 Hz, 1H), 8.10 (dd, J=9.2, 5.8 Hz, 1H), 7.98 (dd, J=11.0, 2.4 Hz, 1H), 7.91 (s, 1H), 7.67 (dd, J=11.6, 5.6 Hz, 1H), 7.62-7.46 (m, 3H), 3.72 (s, 3H), 3.46-3.41 (m, 6.6 Hz, 2H), 2.16-2.03 (m, 2H), 1.96-1.71 (m, 4H), 1.67-1.55 (m, 2H), 1.36 (d, J=6.7 Hz, 3H), 1.17-1.14 (m, 1H).

Example 8 to 10b (Compounds 8 to 10b) were prepared in a procedure similar to Example 7

### Example 8: 2-(1-((1s,4s)-4-(6-fluoroquinolin-4-yl)cyclohexyl)ethyl)-N,N-dimethyl-1H-benzo[d]imidazole-5-carboxamide

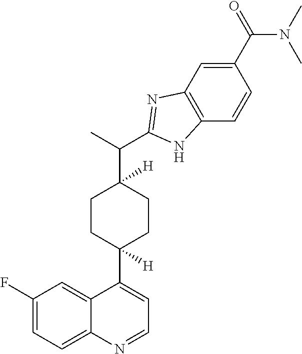 [SMILES: CC(c1nc2cc(C(=O)N(C)C)ccc2[nH]1)[C@H]1CC[C@@H](c2ccnc3ccc(F)cc32)CC1]

¹H NMR (400 MHz, cd3od) δ_{H} 8.69 (d, J=4.7 Hz, 1H), 7.98 (dd, J=9.2, 5.6 Hz, 1H), 7.79 (dd, J=10.6, 2.6 Hz, 1H), 7.62-7.44 (m, 3H), 7.21 (d, J=8.3 Hz, 1H), 3.48-3.28 (m, 2H), 3.03 (s, 3H), 2.96 (s, 3H), 2.16-2.09 (m, 2H), 2.00-1.73 (m, 4H), 1.66-1.60 (m, 2H), 1.37 (d, J=6.8 Hz, 3H), 1.22 (d, J=11.8 Hz, 1H).

### Example 9: N-cyclopropyl-2-((R)-1-((1s,4S)-4-(6-fluoroquinolin-4-yl)cyclohexyl)ethyl)-1H-benzo[d]imidazole-6-carboxamide

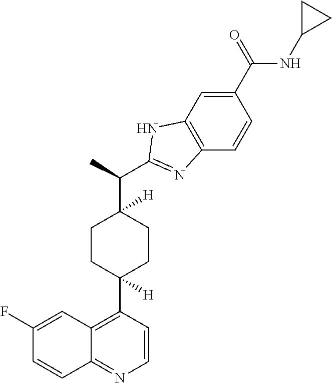 [SMILES: C[C@@H](c1nc2ccc(C(=O)NC3CC3)cc2[nH]1)[C@H]1CC[C@@H](c2ccnc3ccc(F)cc32)CC1]

1H NMR (400 MHz, DMSO-d) δ_{H} 12.42 (d, J=11.4 Hz, 1H), 8.86 (d, J=4.5 Hz, 1H), 8.37-8.33 (m, 1H), 8.10 (dd, J=9.2, 5.8 Hz, 1H), 8.04 (s, 0.5H), 7.98 (dd, J=11.0, 2.7 Hz, 1H), 7.90 (s, 0.5H), 7.70-7.40 (m, 4H), 3.48-3.36 (m, 2H), 2.88-2.83 (m, 1H), 2.16-2.03 (m, 2H), 1.95-1.73 (m, 4H), 1.69-1.51 (m, 2H), 1.36 (d, J=6.8 Hz, 3H), 1.17-1.14 (m, 1H), 0.69-0.68 (m, 2H), 0.59-0.57 (m, 2H).

### Example 10: N-cyclobutyl-2-(1-((1s,4s)-4-(6-fluoroquinolin-4-yl)cyclohexyl)ethyl)-7-methyl-1H-benzo[d]imidazole-5-carboxamide

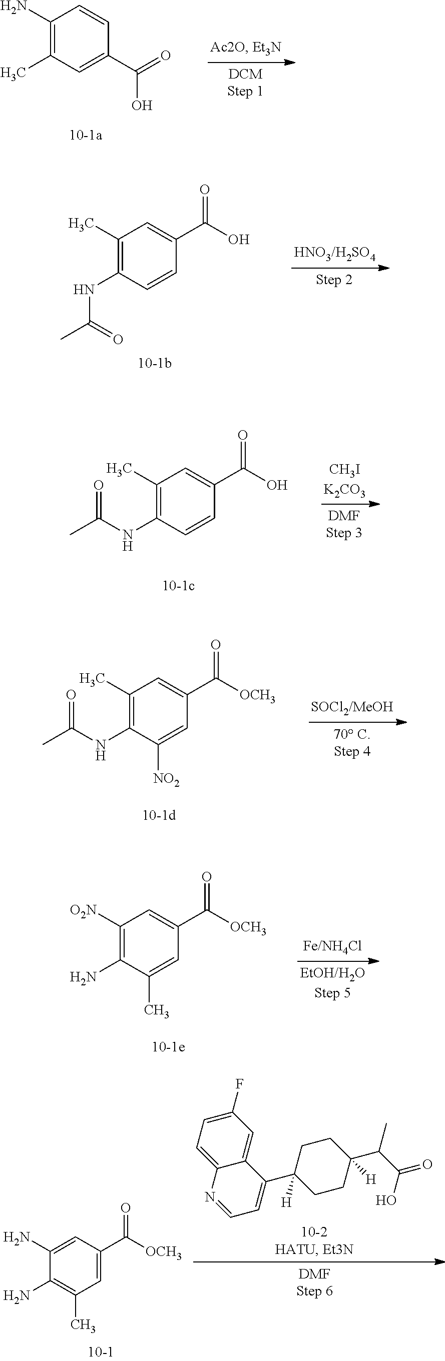

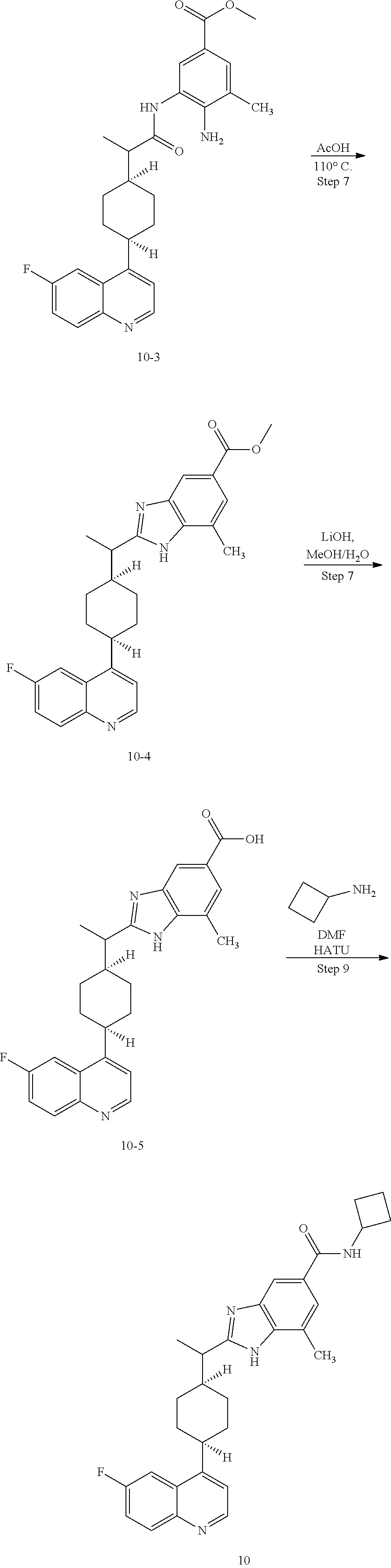

### Step 1: 4-acetamido-3-methylbenzoic Acid

To a solution of 4-amino-3-methylbenzoic acid (10 g, 66 mmol) in DCM (50 mL) were added Et₃N (13 g) and Ac₂O (8 g, 8 mL) at 0° C. The mixture was stirred at room temperature for overnight. The solvent was removed under vacuum. To the mixture was added H₂O (50 ml) and the mixture was extracted with EA (50 mL*2). The organic phase was separated, washed with brine (100 mL). and concentrated to give 4-acetamido-3-methylbenzoic acid (10 g) as a white solid for next step directly without further purification.

### Step 2: 4-acetamido-3-methyl-5-nitrobenzoic Acid

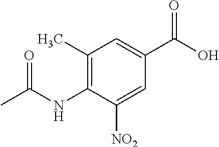 [SMILES: CC(=O)Nc1c(C)cc(C(=O)O)cc1[N+](=O)[O-]]

HNO₃ (20 ml) was slowly dropwised into H₂SO₄ (20 ml) at 0° C. The mixture was stirred for 10 mins at this temperature, then 4-acetamido-3-methylbenzoic acid (10 g) was added into the mixture and stirred for another 1 h at 0° C. After the reaction was completed, the mixture was dropped into ice under stirring. The solid was filtered and dried to give 4-acetamido-3-methyl-5-nitrobenzoic acid (9 g).

### Step 3: methyl 4-acetamido-3-methyl-5-nitrobenzoate

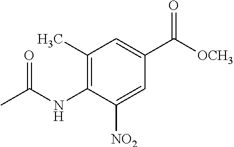 [SMILES: COC(=O)c1cc(C)c(NC(C)=O)c([N+](=O)[O-])c1]

To a solution of 4-acetamido-3-methyl-5-nitrobenzoic acid (5 g, 21 mmol) in DMF (20 ml) was added CH₃I (2.98 g, 1.2 ml) and K₂CO₃ (5.8 g, 42 mmol). The mixture was stirred overnight at room temperature. To the mixture was added H₂O (60 ml) and the mixture was extracted with EA (60 mL*2). The organic layer was dried over with Na₂SO₄, filtered and concentrated to give crude product which was further purified by combiflash, eluting with EA:PE=1:0 to 1:1 to give methyl 4-acetamido-3-methyl-5-nitrobenzoate (3.7 g).

### Step 4: methyl 4-amino-3-methyl-5-nitrobenzoate

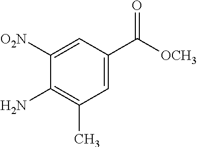

To a solution of methyl 4-acetamido-3-methyl-5-nitrobenzoate (3.7 g) in MeOH (50 ml) was added SOCl₂ (6 ml). The mixture was stirred overnight at 65° C. The solvent was removed under vacuum. The residue was basified to pH>7 with Na₂CO₃ (aq.) and extracted with EA (50 mL*3). The organic layer was dried over with Na₂SO₄, filtered and concentrated to give methyl 4-amino-3-methyl-5-nitrobenzoate (3.4 g) which was used next step without further purification.

### Step 5: methyl 3,4-diamino-5-methylbenzoate

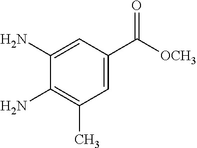

To a suspension of methyl 4-amino-3-methyl-5-nitrobenzoate (3.4 g, 16.2 mmol) in EtOH (60 ml) and H₂O (10 ml) was added iron powder (4.5 g, 87 mmol) and NH₄Cl (6.9 g, 129.6 mmol). The mixture was stirred for 8 h at 70° C. under N₂. The mixture was filtered and the filtrate was concentrated. To the mixture was added NaHCO₃ (aq. 50 ml) and the mixture was extracted with EA (50 mL*2). The organic layer was dried over with Na₂SO₄, filtered and concentrated to give methyl 3,4-diamino-5-methylbenzoate (2.0 g) which was used next step without further purification.

### Step 6: methyl 4-amino-3-(2-((1s,4s)-4-(6-fluoroquinolin-4-yl)cyclohexyl)propanamido)-5-methylbenzoate

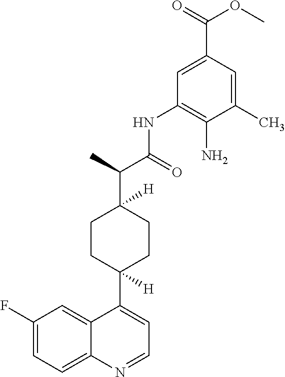 [SMILES: COC(=O)c1cc(C)c(N)c(NC(=O)[C@H](C)[C@H]2CC[C@@H](c3ccnc4ccc(F)cc43)CC2)c1]

TO a solution of (R)-2-((1s,4S)-4-(6-fluoroquinolin-4-yl)cyclohexyl)propanoic acid (301 mg, 1.0 mmol) in DMF (20 mL) were added methyl 3,4-diamino-5-methylbenzoate (217 mg, 1.2 mmol), HATU (456 mg, 1.2 mmol), and Et₃N (204 mg, 2.0 mmol), the mixture was stirred overnight at room temperature. After the reaction was completed, EA was added, the mixture was washed with water and brine, dried over Na₂SO₄, and concentrated to methyl 4-amino-3-(2-((1s,4s)-4-(6-fluoroquinolin-4-yl)cyclohexyl)propanamido)-5-methylbenzoate (400 mg) which was used next step without further purification.

### Step 7: methyl 2-(1-((1s,4s)-4-(6-fluoroquinolin-4-yl)cyclohexyl)ethyl)-7-methyl-1H-benzo[d]imidazole-5-carboxylate

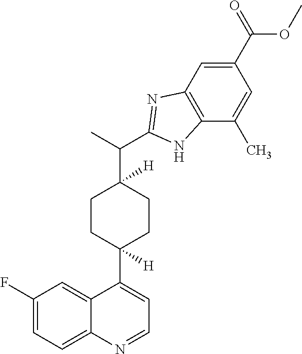 [SMILES: COC(=O)c1cc(C)c2[nH]c(C(C)[C@H]3CC[C@@H](c4ccnc5ccc(F)cc54)CC3)nc2c1]

A solution of methyl 4-amino-3-(2-((1s,4s)-4-(6-fluoroquinolin-4-yl)cyclohexyl)propanamido)-5-methylbenzoate (400 mg) in HOAc (20 mL) was heated to 110° C. for 8 hours, after cooled down, the mixture was concentrated, with saturated NaHCO₃.aq added, and extracted with EA, the EA layer was washed with brine, dried over Na₂SO₄, concentrated and purified by sili-gel to give methyl 2-(1-((1s,4s)-4-(6-fluoroquinolin-4-yl)cyclohexyl)ethyl)-7-methyl-1H-benzo[d]imidazole-5-carboxylate (120 mg).

### Step 3: 2-(1-((1s,4s)-4-(6-fluoroquinolin-4-yl)cyclohexyl)ethyl)-7-methyl-1H-benzo[d]imidazole-5-carboxylic acid

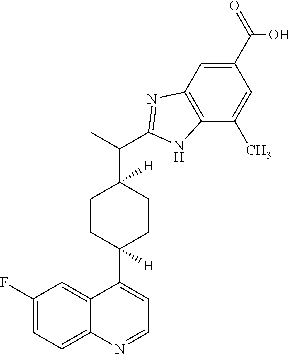 [SMILES: Cc1cc(C(=O)O)cc2nc(C(C)[C@H]3CC[C@@H](c4ccnc5ccc(F)cc54)CC3)[nH]c12]

A solution of methyl 2-(1-((1s,4s)-4-(6-fluoroquinolin-4-yl)cyclohexyl)ethyl)-7-methyl-1H-benzo[d]imidazole-5-carboxylate (110 mg, 0.25 mmol) and LiOH.H₂O (53 mg, 1.25 mmol in MeOH/H₂O (10 mL/2 mL) was stirred for 48 hours at room temperature, the reaction mixture was concentrated, the residue's pH value was adjusted to 6 with 1N HCl.aq, the white solid was collected and dried in vacuo to give 60 mg crude 2-(1-((1s,4s)-4-(6-fluoroquinolin-4-yl)cyclohexyl)ethyl)-7-methyl-1H-benzo[d]imidazole-5-carboxylic acid.

### Step 8: N-cyclobutyl-2-(1-((1s,4s)-4-(6-fluoroquinolin-4 yl)cyclohexyl)ethyl)-7-methyl-1H-benzo[d]imidazole-5-carboxamide

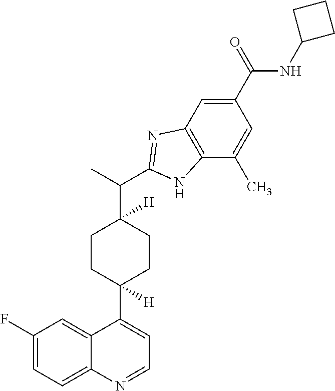 [SMILES: Cc1cc(C(=O)NC2CCC2)cc2nc(C(C)[C@H]3CC[C@@H](c4ccnc5ccc(F)cc54)CC3)[nH]c12]

A solution of 2-(1-((1s,4s)-4-(6-fluoroquinolin-4-yl)cyclohexyl)ethyl)-7-methyl-1H-benzo[d]imidazole-5-carboxylic acid (60 mg, 0.14 mmol), cyclobutanamine (12 mg, 0.168 mmol), HATU (64 mg, 0.168 mmol) and 4-methylmorpholine (0.5 mL) in DMF (10 mL) was stirred for 16 hours at room temperature, the reaction mixture was concentrated, with EA added, washed with water and brine, dried over Na₂SO₄, concentrated and purified by sili-gel to give 50 mg N-cyclobutyl-2-(1-((1s,4s)-4-(6-fluoroquinolin-4-yl)cyclohexyl)ethyl)-7-methyl-1H-benzo[d]imidazole-5-carboxamide. ¹H NMR (400 MHz, DMSO-d₆) δ_{H} 12.33-12.35 (m, 1H), 8.86 (d, J=8.0 Hz, 1H), 8.47 (d, J=8.0 Hz, 1H), 8.08-8.11 (m, 1H), 7.96-7.99 (m, 1H), 7.65-7.69 (m, 2H), 7.58-7.59 (m, 1H), 7.48 (s, 1H), 4.39-4.46 (m, 1H), 3.41-3.48 (m, 2H), 2.52 (s, 3H), 2.15-2.20 (m, 3H), 2.05-2.09 (m, 3H), 1.77-1.93 (m, 4H), 1.55-1.66 (m, 4H), 1.36 (d, J=8.0 Hz, 3H), and 1.16-1.23 (m, 1H). [M+H]+=485.

### Examples 10a and 10b: N-cyclobutyl-2-((R)-1-((1s,4S)-4-(6-fluoroquinolin-4-yl)cyclohexyl)ethyl)-7-methyl-1H-benzo[d]imidazole-5-carboxamide and N-cyclobutyl-2-((S)-1-((1s,4R)-4-(6-fluoroquinolin-4 yl)cyclohexyl)ethyl)-7-methyl-1H-benzo[d]imidazole-5-carboxamide

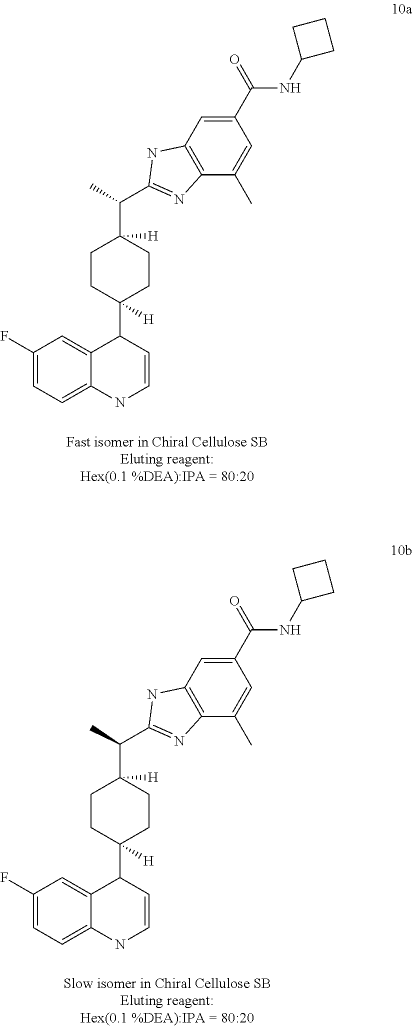 [SMILES: Cc1cc(C(=O)NC2CCC2)cc2[nH]c([C@@H](C)[C@H]3CC[C@@H](C4C=CNc5ccc(F)cc54)CC3)nc12 ; Cc1cc(C(=O)NC2CCC2)cc2[nH]c([C@H](C)[C@H]3CC[C@@H](C4C=CNc5ccc(F)cc54)CC3)nc12]

Compounds 10a and 10b were separated using preparative HPLC on a CHIRAL ART Cellulose-SB with HEX (8 mmol/L NH₃.MeOH):IPA=80:20 as an eluent. The first one enantiomer eluted at the retention time of 3.683 min, 10a (6.82 mg), ¹H NMR (DMSO-d₆) δ_{H} 12.26-12.34 (m, 1H), 8.86 (d, J=8.0 Hz, 1H), 8.46-8.48 (m, 1H), 8.08-8.11 (m, 1H), 7.96-7.99 (m, 1H), 7.74-7.90 (m, 1H), 7.59-7.69 (m, 2H), 7.47 (s, 1H), 4.40-4.46 (m, 1H), 3.41-3.44 (m, 2H), 2.52 (s, 3H), 2.15-2.20 (m, 3H), 2.04-2.07 (m, 3H), 1.58-1.89 (m, 8H), 1.36 (d, J=8.0 Hz, 3H), and 1.19-1.29 (m, 1H). [M+H]+=485. And the other enantiomer eluted at the retention time of 4.778 min, 10b (10.33 mg), ¹H NMR (DMSO-d₆) δ_{H} 12.26-12.34 (m, 1H), 8.87 (d, J=8.0 Hz, 1H), 8.46-8.48 (m, 1H), 8.08-8.11 (m, 1H), 7.96-7.99 (m, 1H), 7.74-7.90 (m, 1H), 7.59-7.69 (m, 2H), 7.47 (s, 1H), 4.42-4.46 (m, 1H), 3.41-3.44 (m, 2H), 2.52 (s, 3H), 2.15-2.20 (m, 3H), 2.04-2.07 (m, 3H), 1.58-1.89 (m, 8H), 1.36 (d, J=8.0 Hz, 3H), and 1.17-1.24 (m, 1H). [M+H]+=485.

### Example 11: 2-(1-(4-(6-fluoroquinolin-4-yl)cyclohexyl)ethyl)-N-(oxetan-3-yl)-1H-benzo[d]imidazole-6-carboxamide

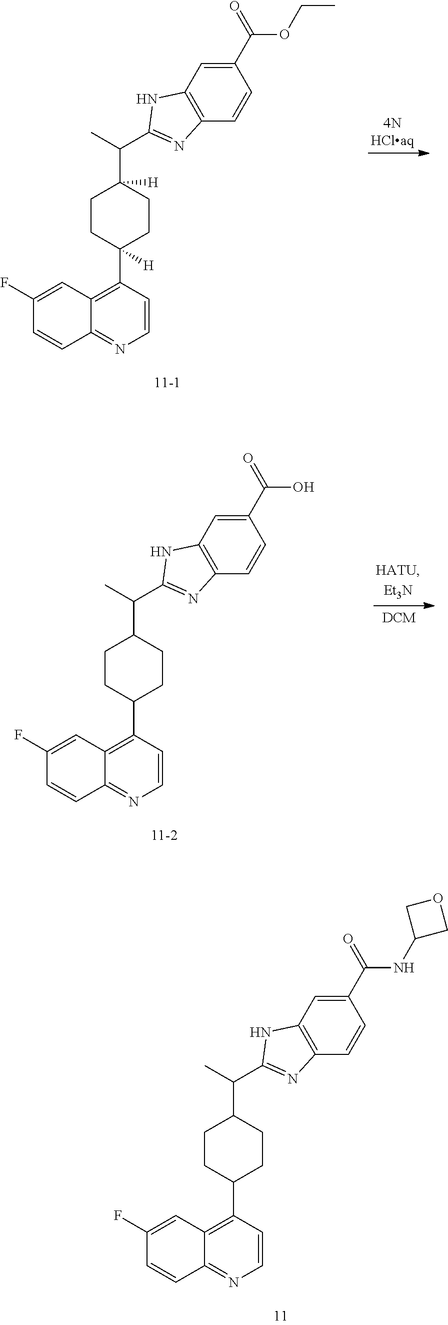 [SMILES: CCOC(=O)c1ccc2nc(C(C)[C@H]3CC[C@@H](c4ccnc5ccc(F)cc54)CC3)[nH]c2c1 ; CC(c1nc2ccc(C(=O)O)cc2[nH]1)C1CCC(c2ccnc3ccc(F)cc23)CC1 ; CC(c1nc2ccc(C(=O)NC3COC3)cc2[nH]1)C1CCC(c2ccnc3ccc(F)cc23)CC1]

### Step 1: 2-(1-(4-(6-fluoroquinolin-4-yl)cyclohexyl)ethyl)-1H-benzo[d]imidazole-6-carboxylic acid

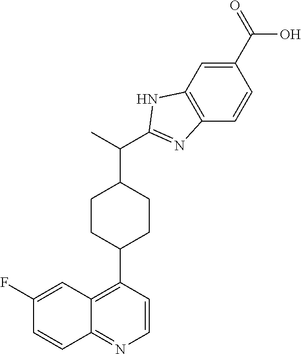 [SMILES: CC(c1nc2ccc(C(=O)O)cc2[nH]1)C1CCC(c2ccnc3ccc(F)cc23)CC1]

A solution of ethyl ethyl 2-(1-((1s,4s)-4-(6-fluoroquinolin-4-yl)cyclohexyl)ethyl)-1H-benzo[d]imidazole-6-carboxylate (5.0 g, 11.2 mmol) in 4N HCl.aq (150 mL) was heated to 80° C. for 5 hours, the reaction mixture was concentrated, the residue's pH value was adjusted to 6 with saturated.NaHCO₃.aq, extracted with EA, the EA layer was concentrated and purified by sili-gel to give 2.0 g 2-(1-(4-(6-fluoroquinolin-4-yl)cyclohexyl)ethyl)-1H-benzo[d]imidazole-6-carboxylic acid.

### Step 2: 2-(1-(4-(6-fluoroquinolin-4-yl)cyclohexyl)ethyl)-N-(oxetan-3-yl)-1H-benzo[d]imidazole-6-carboxamide

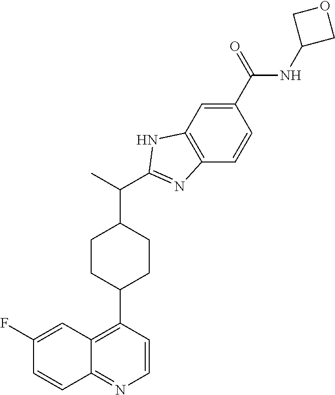 [SMILES: CC(c1nc2ccc(C(=O)NC3COC3)cc2[nH]1)C1CCC(c2ccnc3ccc(F)cc23)CC1]

A solution of 2-(1-(4-(6-fluoroquinolin-4-yl)cyclohexyl)ethyl)-1H-benzo[d]imidazole-6-carboxylic acid (100 mg, 0.24 mmol), oxetan-3-amine (36 mg, 0.5 mmol), HATU (150 mg, 0.39 mmol) and Et₃N (100 mg, 1.0 mmol) in DCM (10 mL) was stirred for 16 hours at room temperature, the reaction mixture was concentrated, EA was added, washed with water and brine, dried over Na₂SO₄, concentrated and purified by sill-gel and prep-HPLC to give desired product. ¹H NMR (400 MHz, DMSO-d) δ_{H} 12.47 (d, J=13.0 Hz, 1H), 9.01 (dd, J=19.5, 6.3 Hz, 1H), 8.87 (d, J=4.5 Hz, 1H), 8.16-7.95 (m, 3H), 7.68 (m, 2H), 7.53 (m, 2H), 5.03 (m, 1H), 4.77 (t, J=6.7 Hz, 2H), 4.62 (td, J=6.4, 2.5 Hz, 2H), 3.43 (m, 2H), 2.19-1.53 (m, 8H), 1.37 (d, J=6.8 Hz, 3H), 1.17 (in, 1H).

### Example 12: 2-((R)-1-((1r,4R)-4-(6-fluoroquinolin-4-yl)cyclohexyl)ethyl)-N-(2-methoxyethyl)-1H-benzo[d]imidazole-6-carboxamide

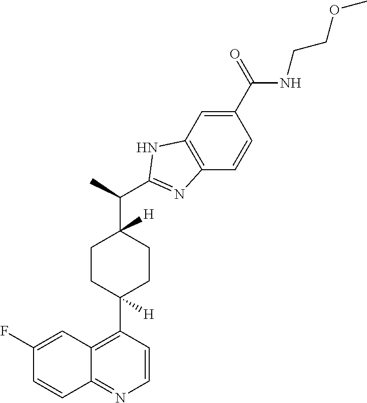 [SMILES: COCCNC(=O)c1ccc2nc([C@H](C)[C@H]3CC[C@H](c4ccnc5ccc(F)cc54)CC3)[nH]c2c1]

¹H NMR (400 MHz, DMSO-d) δ_{H} 12.39 (d, J=13.0 Hz, 1H), 8.79 (d, J=4.6 Hz, 1H), 8.43 (m, 1H), 8.02 (m, 3H), 7.72-7.38 (m, 4H), 3.52-3.40 (m, 4H), 3.28 (s, 3H), 2.94 (m, 1H), 1.98-1.41 (m, 8H), 1.40 (d, J=7.2 Hz, 3H).

### Example 13: 2-((R)-1-((1s,4S)-4-(6-fluoroquinolin-4-yl)cyclohexyl)ethyl)-N-(2-methoxyethyl)-1H-benzo[d]imidazole-6-carboxamide

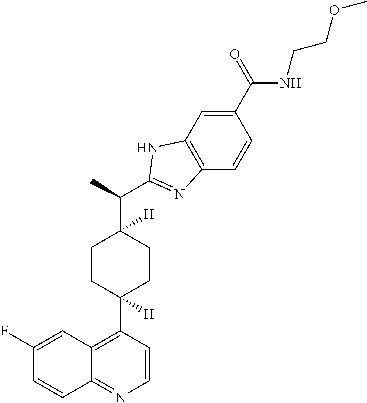 [SMILES: COCCNC(=O)c1ccc2nc([C@H](C)[C@H]3CC[C@@H](c4ccnc5ccc(F)cc54)CC3)[nH]c2c1]

¹H NMR (400 MHz, DMSO-d) δ_{H} 12.43 (d, J=13.3 Hz, 1H), 8.86 (d, J=4.5 Hz, 1H), 8.42 (m, 1H), 8.14-7.91 (m, 3H), 7.71-7.42 (m, 4H), 3.49-3.37 (m, 6H), 3.27 (s, 3H), 2.10 (m, 2H), 1.95-1.53 (m, 6H), 1.36 (d, J=6.8 Hz, 3H), 1.16 (m, 1H).

### Example 14: 2-((R)-1-((1s,4S)-4-(6-fluoroquinolin-4-yl)cyclohexyl)ethyl)-N phenyl-1H-benzo[d]imidazole-6-carboxamide

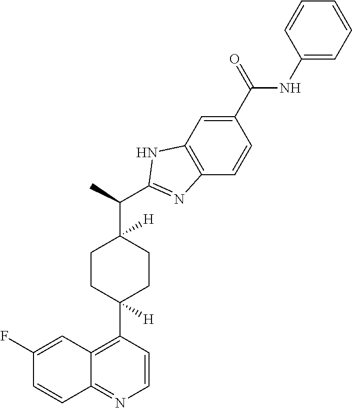 [SMILES: C[C@@H](c1nc2ccc(C(=O)Nc3ccccc3)cc2[nH]1)[C@H]1CC[C@@H](c2ccnc3ccc(F)cc32)CC1]

¹H NMR (400 MHz, DMSO-d) δ_{H} 12.50 (d, J=17.1 Hz, 1H), 10.19 (d, J=19.7 Hz, 1H), 8.80 (d, J=4.5 Hz, 1H), 8.30-7.94 (m, 3H), 7.81 (m, 3H), 7.60 (m, 2H), 7.44 (d, J=4.7 Hz, 1H), 7.35 (t, J=7.7 Hz, 2H), 7.09 (t, J=7.3 Hz, 1H), 3.01-2.91 (m, 1H), 2.00-1.23 (m, 12H).

### Example 15: 2-((R)-1-((1r,4R)-4-(6-fluoroquinolin-4-yl)cyclohexyl)ethyl)-N-phenyl-1H-benzo[d]imidazole-6-carboxamide

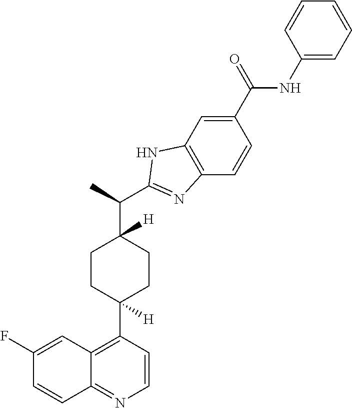 [SMILES: C[C@@H](c1nc2ccc(C(=O)Nc3ccccc3)cc2[nH]1)[C@H]1CC[C@H](c2ccnc3ccc(F)cc32)CC1]

¹H NMR (400 MHz, DMSO-d) δ_{H} 12.53 (d, J=20.5 Hz, 1H), 10.18 (d, J=16.3 Hz, 1H), 8.87 (d, J=4.4 Hz, 1H), 8.28-7.93 (m, 3H), 7.84-7.73 (m, 3H), 7.71-7.50 (m, 3H), 7.34 (t, J=7.5 Hz, 2H), 7.08 (t, J=7.2 Hz, 1H), 3.44 (m, 2H), 2.22-1.52 (m, 8H), 1.38 (d, J=6.7 Hz, 3H), 1.23 (m, 1H).

### Example 16: 2-((R)-1-((1s,4S)-4-(6-fluoroquinolin-4-yl)cyclohexyl)ethyl)-N-((1-hydroxycyclobutyl)methyl)-1H-benzo[d]imidazole-6-carboxamide

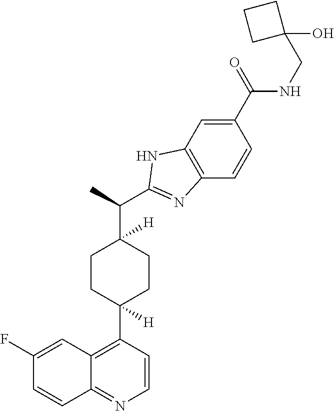 [SMILES: C[C@@H](c1nc2ccc(C(=O)NCC3(O)CCC3)cc2[nH]1)[C@H]1CC[C@@H](c2ccnc3ccc(F)cc32)CC1]

¹H NMR (400 MHz, DMSO-d) δ_{H} 12.32 (s, 1H), 8.79 (d, J=4.6 Hz, 1H), 8.07 (dd, J=9.0, 5.9 Hz, 1H), 7.98 (m, 2H), 7.66 (1n, 2H), 7.43 (m, 2H), 4.90 (s, 1H), 4.17 (d, J=10.8 Hz, 1H), 3.98 (d, J=8.8 Hz, 1H), 3.55 (d, J=16.7 Hz, 1H), 2.98-2.87 (m, 2H), 1.95-1.50 (m, 6H), 1.38 (d, J=6.8 Hz, 3H)

### Example 17: 2-((R)-1-((1 r,4R)-4-(6-fluoroquinolin-4-yl)cyclohexyl)ethyl)-N-((1-hydroxycyclobutyl)methyl)-1H-benzo[d]imidazole-6-carboxamide

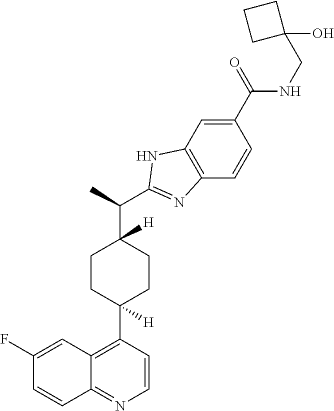 [SMILES: C[C@@H](c1nc2ccc(C(=O)NCC3(O)CCC3)cc2[nH]1)[C@H]1CC[C@H](c2ccnc3ccc(F)cc32)CC1]

¹H NMR (400 MHz, DMSO-d) δ_{H} 12.46 (s, 1H), 8.85 (d, J=4.3 Hz, 1H), 8.09 (dd, J=9.0, 5.9 Hz, 1H), 8.03-7.89 (m, 2H), 7.76-7.61 (m, 2H), 7.57 (d, J=4.0 Hz, 1H), 7.45 (dd, J=35.0, 8.4 Hz, 1H), 4.96-4.84 (m, 2H), 4.17 (d, J=10.6 Hz, 1H), 3.98 (d, J=10.7 Hz, 1H), 3.55 (d, J=16.8 Hz, 1H), 3.47-3.39 (m, 2H), 3.34 (d, J=5.4 Hz, 2H), 3.25 (d, J=16.9 Hz, 1H), 2.09 (m, 2H), 1.95-1.50 (m, 6H), 1.35 (d, J=6.6 Hz, 3H), 1.17 (m, 1H).

### Example 18: 2-((R)-1-((1s,4S)-4-(6-fluoroquinolin-4-yl)cyclohexyl)ethyl)-N-(4-methoxycyclohexyl)-1H-benzo[d]imidazole-6-carboxamide

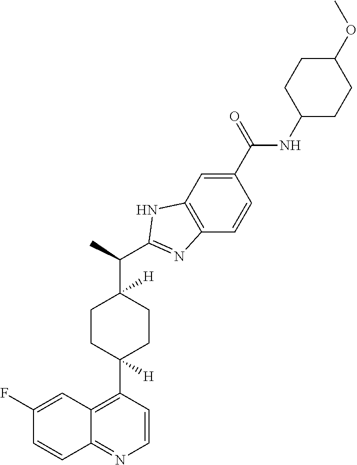 [SMILES: COC1CCC(NC(=O)c2ccc3nc([C@H](C)[C@H]4CC[C@@H](c5ccnc6ccc(F)cc65)CC4)[nH]c3c2)CC1]

¹H NMR (400 MHz, DMSO-d) δ_{H} 12.41 (ds, 1H), 8.87 (d, J=4.5 Hz, 1H), 8.17-7.90 (m, 4H), 7.70-7.42 (m, 4H), 3.77 (m, 1H), 3.42 (m, 2H), 3.24 (s, 3H), 3.11 (m, 1H), 2.18-1.52 (m, 14H), 1.36 (d, J=6.6 Hz, 3H), 1.23 (m, 1H).

### Example 19: 2-((R)-1-((1r,4R)-4-(6-fluoroquinolin-4-yl)cyclohexyl)ethyl)-N-(4-methoxycyclohexyl)-1H-benzo[d]imidazole-6-carboxamide

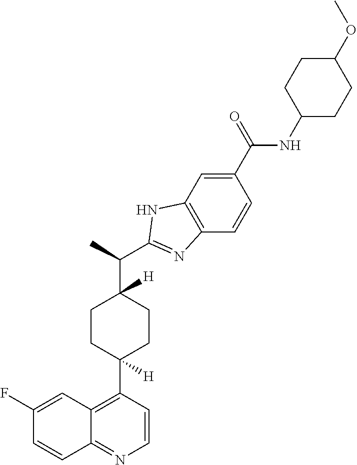 [SMILES: COC1CCC(NC(=O)c2ccc3nc([C@H](C)[C@H]4CC[C@H](c5ccnc6ccc(F)cc65)CC4)[nH]c3c2)CC1]

¹H NMR (400 MHz, DMSO-d) δ_{H} 12.41 (ds, 1H), 8.87 (d, J=4.5 Hz, 1H), 8.17-7.90 (m, 4H), 7.70-7.42 (m, 4H), 3.77 (m, 1H), 3.42 (m, 2H), 3.24 (s, 3H), 3.11 (m, 1H), 2.18-1.52 (m, 14H), 1.36 (d, J=6.6 Hz, 3H), 1.23 (m, 1H).

### Example 20: N-cyclopropyl-6-fluoro-2-(1-((1s,4S)-4-(6-fluoroquinolin-4-yl)cyclohexyl)ethyl)-1H-benzo[d]imidazole-5-carboxamide

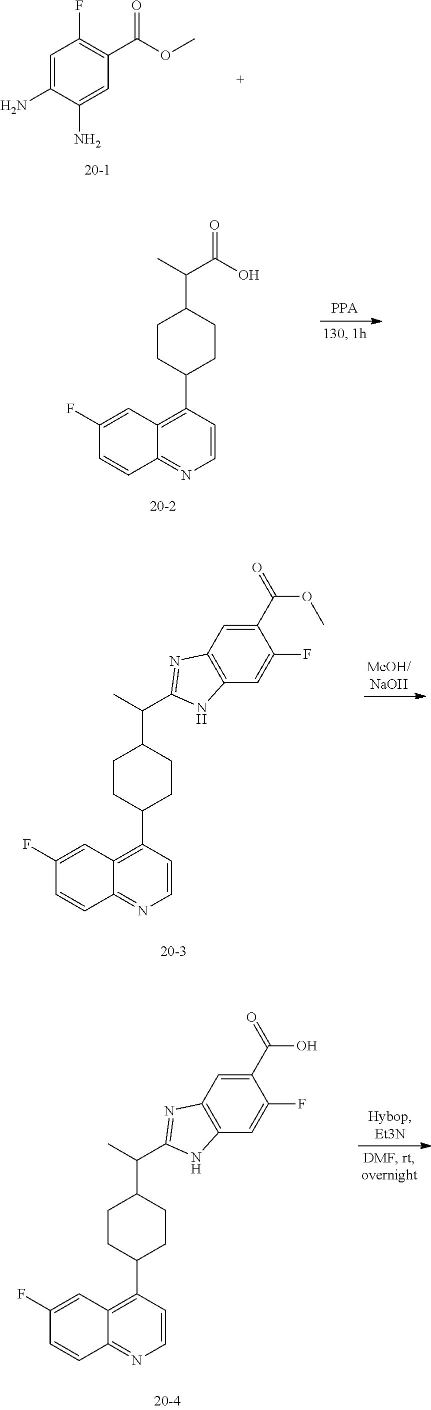 [SMILES: COC(=O)c1cc(N)c(N)cc1F ; CC(C(=O)O)C1CCC(c2ccnc3ccc(F)cc23)CC1 ; COC(=O)c1cc2nc(C(C)C3CCC(c4ccnc5ccc(F)cc45)CC3)[nH]c2cc1F ; CC(c1nc2cc(C(=O)O)c(F)cc2[nH]1)C1CCC(c2ccnc3ccc(F)cc23)CC1]

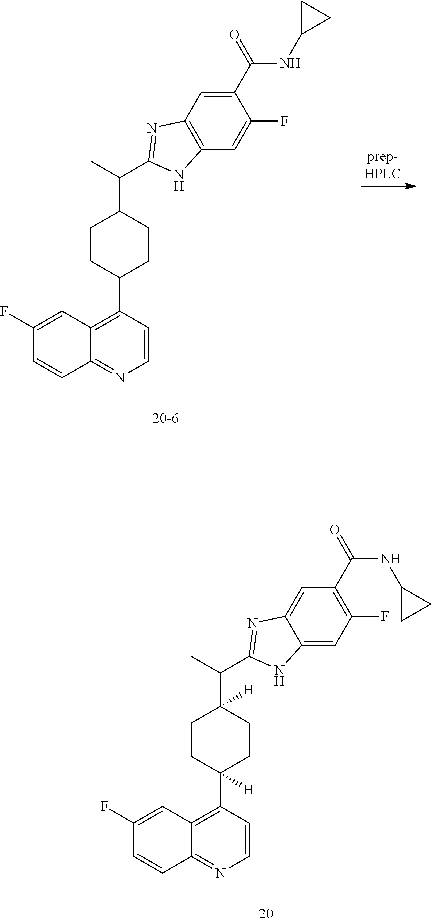 [SMILES: CC(c1nc2cc(C(=O)NC3CC3)c(F)cc2[nH]1)C1CCC(c2ccnc3ccc(F)cc23)CC1 ; CC(c1nc2cc(C(=O)NC3CC3)c(F)cc2[nH]1)[C@H]1CC[C@@H](c2ccnc3ccc(F)cc32)CC1]

### Step 1: Methyl6-fluoro-2-(1-(4-(6-fluoroquinolin-4-yl)cyclohexyl)ethyl)-1H-benzo[d]imidazole-5-carboxylate

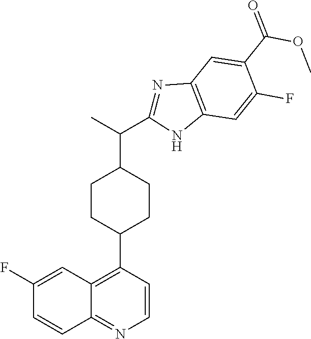 [SMILES: COC(=O)c1cc2nc(C(C)C3CCC(c4ccnc5ccc(F)cc45)CC3)[nH]c2cc1F]

A mixture of 2-((1s,4S)-4-(6-fluoroquinolin-4-yl)cyclohexyl)propanoic acid (600 mg, 2.0 mmol) and methyl 4,5-diamino-2-fluorobenzoate (360 mg, 2.0 mmol) in PPA (15 mL) was heated to 130° C. for 2 hours. The mixture reaction was poured into NaOH.aq (5%, 100 mL), extracted with EA, the EA layer was dried over Na₂SO₄, concentrated and purified by sili-gel to give methyl 6-fluoro-2-(1-(4-(6-fluoroquinolin-4-yl)cyclohexyl)ethyl)-1H-benzo[d]imidazole-5-carboxylate.

### Step 2: 6-fluoro-2-(1-(4-(6-fluoroquinolin-4 yl)cyclohexyl)ethyl)-1H-benzo[d]imidazole-5-carboxylic Acid

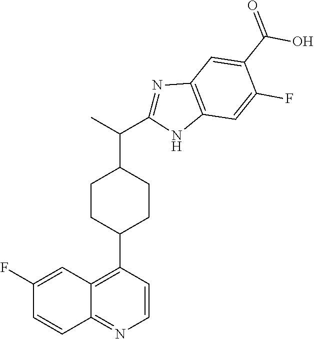 [SMILES: CC(c1nc2cc(C(=O)O)c(F)cc2[nH]1)C1CCC(c2ccnc3ccc(F)cc23)CC1]

A solution of methyl 6-fluoro-2-(1-(4-(6-fluoroquinolin-4-yl)cyclohexyl)ethyl)-1H-benzo[d]imidazole-5-carboxylate (300 mg, 0.67 mmol) and NaOH (200 mg, 5.0 mmol) in MeOH/H₂O (20 mL/20 mL) was stirred for 48 hours at room temperature, the reaction mixture was concentrated, the residue's pH value was adjusted to 5 with 1N HCl.aq, the white solid was collected and dried in vacuo to give 6-fluoro-2-(1-(4-(6-fluoroquinolin-4-yl)cyclohexyl)ethyl)-1H-benzo[d]imidazole-5-carboxylic acid.

### Step 3: N-cyclopropyl-6-fluoro-2-(1-(4-(6-fluoroquinolin-4-yl)cyclohexyl)ethyl)-1H-benzo[d]imidazole-5-carboxamide

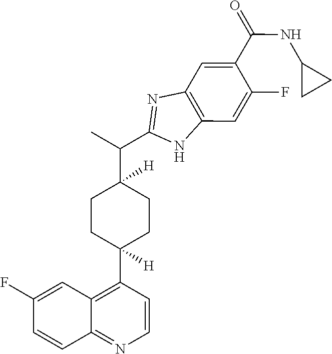 [SMILES: CC(c1nc2cc(C(=O)NC3CC3)c(F)cc2[nH]1)[C@H]1CC[C@@H](c2ccnc3ccc(F)cc32)CC1]

A solution of 6-fluoro-2-(1-(4-(6-fluoroquinolin-4-yl)cyclohexyl)ethyl)-1H-benzo[d]imidazole-5-carboxylic acid (80 mg, 0.17 mmol), cyclopropanamine (15 mg, 0.2 mmol), Hybop (140 mg, 0.27 mmol) and Et₃N (150 mg, 1.5 mmol) in DMF (8 mL) was stirred for 16 hours at room temperature, EA was added, washed with water and brine, dried over Na₂SO4, concentrated and purified by sill-gel and prep-HPLC to give N-cyclopropyl-6-fluoro-2-(1-(4-(6-fluoroquinolin-4-yl)cyclohexyl)ethyl)-1H-benzo[d]imidazole-5-carboxamide. ¹H NMR (400 MHz, DMSO-d) 8H 12.48 (d, 1H), 8.86 (d, J=4.4 Hz, 1H), 8.20 (s, 1H), 8.09 (m, 1H), 7.97 (d, J=10.8 Hz, 1H), 7.55-7.70 (m, 3H), 7.27-7.39 (dd, 1H), 3.42 (m, 2H), 2.85 (m, 1H), 1.54-2.14 (m, 9H), 1.34 (d, J=5.6 Hz, 3H), 0.68 (m, 2H), 0.55 (m, 2H).

### Example 21: N-cyclopropyl-4,5-difluoro-2-(1-((1s,4s)-4-(6-fluoroquinolin-4-yl)cyclohexyl)ethyl)-1H-benzo[d]imidazole-6-carboxamide

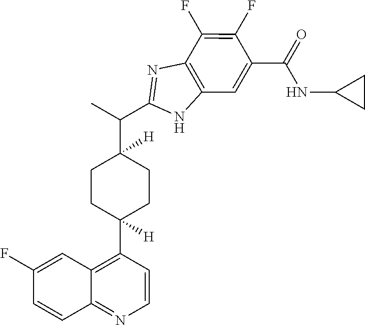 [SMILES: CC(c1nc2c(F)c(F)c(C(=O)NC3CC3)cc2[nH]1)[C@H]1CC[C@@H](c2ccnc3ccc(F)cc32)CC1]

¹H NMR (400 MHz, DMSO-d) δ_{H} 12.8 (s, 1H), 8.86 (m, 1H), 8.37 (m, 1H), 8.10 (m, 1H), 7.98 (m, 1H), 6.66 (m, 1H), 7.59 (m, 1H), 7.42 (m, 1H), 3.42 (m, 2H), 2.85 (m, 1H), 1.55-2.16 (m, 10H), 1.36 (d, J=6.8 Hz, 3H), 0.70 (m, 2H), 0.55 (m, 2H).

### Example 22: N-cyclopropyl-2-(1-((1s,4s)-4-(6-fluoroquinolin-4-yl)cyclohexyl)ethyl)-1H-imidazo[4,5-c]pyridine-6-carboxamide

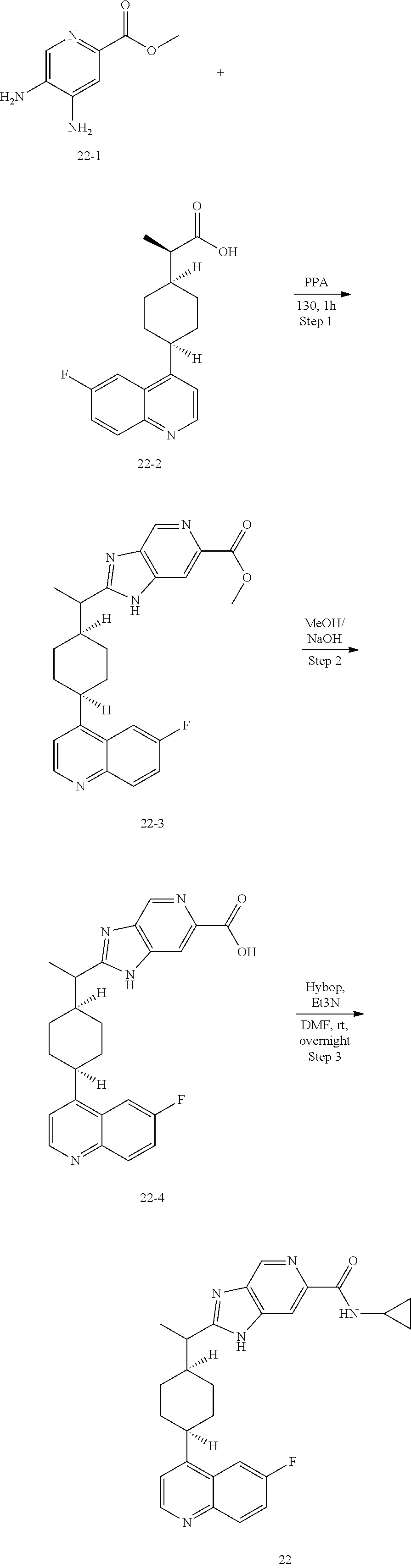 [SMILES: COC(=O)c1cc(N)c(N)cn1 ; C[C@@H](C(=O)O)[C@H]1CC[C@@H](c2ccnc3ccc(F)cc32)CC1 ; COC(=O)c1cc2[nH]c(C(C)[C@H]3CC[C@@H](c4ccnc5ccc(F)cc54)CC3)nc2cn1 ; CC(c1nc2cnc(C(=O)O)cc2[nH]1)[C@H]1CC[C@@H](c2ccnc3ccc(F)cc32)CC1 ; CC(c1nc2cnc(C(=O)NC3CC3)cc2[nH]1)[C@H]1CC[C@@H](c2ccnc3ccc(F)cc32)CC1]

### Step 1: methyl 2-(1-((1s,4S)-4-(6-fluoroquinolin-4-yl)cyclohexyl)ethyl)-1H-imidazo[4,5-c]pyridine-6-carboxylate

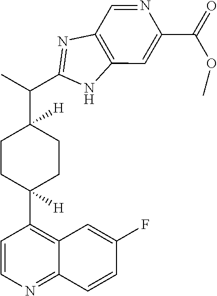 [SMILES: COC(=O)c1cc2[nH]c(C(C)[C@H]3CC[C@@H](c4ccnc5ccc(F)cc54)CC3)nc2cn1]

A mixture of (R)-2-((1s,4S)-4-(6-fluoroquinolin-4-yl)cyclohexyl)propanoic acid (200 mg, 0.664 mmol) and methyl 4,5-diaminopicolinate (130 mg, 0.863 mmol) in PPA (5 ml), was stirred overnight at 125° C. After determining the reaction to be completed by LCMS, the mixture was added H₂O (100 ml). Adjust the pH value of residue to 7-8 with saturated aqueous of NaHCO₃, filtered to give the crude product (276 mg) as yellow solid, within which 20 mg was purified by prep-HPLC, to afford 7.41 mg desired product as a white solid. ¹H NMR (400 MHz, DMSO) δ 13.07 (d, J=20.0 Hz, 1H), 9.00-8.78 (m, 2H), 8.22 (d, J=32.0 Hz, 1H), 8.09 (dd, J=9.2, 6.0 Hz, 1H), 7.98 (d, J=8.8 Hz, 1H), 7.71-7.63 m, 1H), 7.59 (d, J=4.4 Hz, 1H), 3.88 (s, 3H), 3.53 (dd, J=10.4, 6.8 Hz, 1H), 3.43 (s, 1H), 2.18 (d, J=10.0 Hz, 1H), 2.06 (d, J=10.8 Hz, 1H), 1.95-1.71 (m, 4H), 1.71-1.52 (m, 2H), 1.38 (d, J=6.4 Hz, 2H), 1.14 (d, J=10.0 Hz, 1H). [M+1]⁺357.

### Step 2: 2-((R)-1-((1s,4S)-4-(6-fluoroquinolin-4-yl)cyclohexyl)ethyl)-1H-imidazo[4,5-c]pyridine-6-carboxylic acid

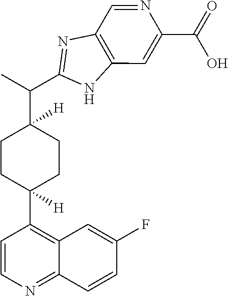 [SMILES: CC(c1nc2cnc(C(=O)O)cc2[nH]1)[C@H]1CC[C@@H](c2ccnc3ccc(F)cc32)CC1]

To a solution of methyl 2-((R)-1-((1s,4S)-4-(6-fluoroquinolin-4-yl)cyclohexyl)-ethyl)-1H-imidazo[4,5-c]pyridine-6-carboxylate (250 mg, 0.578 mmol) in EtOH (5 ml) was added KOH (64.8 mg, 1.157 mmol) and water (3 ml). The mixture was stirred for 2 h at 80° C. After determining the reaction to be completed by LCMS, EtOH was removed under vacuo, and 10 ml water was added. Adjust the pH value of residue aqueous layer to 6 with HCl (1 M), filtered to afford 208 mg desired product as a yellow solid. ¹H NMR (400 MHz, dmso) δ 8.84 (d, J=3.6 Hz, 2H), 8.19 (s, 1H), 8.09 (dd, J=9.2, 6.0 Hz, 1H), 7.98 (dd, J=11.2, 2.4 Hz, 1H), 7.70-7.62 (m, 1H), 7.59 (d, J=4.0 Hz, 1H), 3.54 (dd, J=10.4, 6.8 Hz, 2H), 3.42 (s, 2H), 2.19 (d, J=9.2 Hz, 1H), 2.06 (d, J=12.0 Hz, 1H), 1.96-1.72 (m, 4H), 1.71-1.50 (m, 2H), 1.38 (d, J=6.8 Hz, 3H), 1.15 (d, J=12.8 Hz, 1H). [M+1]⁺419.

### Step 3: N-cyclopropyl-2-(1-((1s,4s)-4-(6-fluoroquinolin-4 yl)cyclohexyl)ethyl)-1H-imidazo[4,5-c]pyridine-6-carboxamide

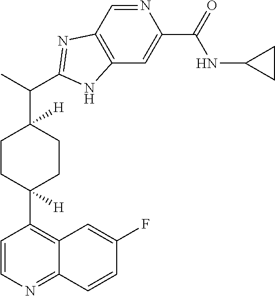 [SMILES: CC(c1nc2cnc(C(=O)NC3CC3)cc2[nH]1)[C@H]1CC[C@@H](c2ccnc3ccc(F)cc32)CC1]

To a solution of 2-((R)-1-((1s,4S)-4-(6-fluoroquinolin-4-yl)cyclohexyl)ethyl)-1H-imidazo[4,5-c]pyridine-6-carboxylic acid (100 mg, 0.239 mmol) in DCM (5 mL) were added HATU (109 mg, 0.289 mmol), TEA (48.3 mg, 0.487 mmol) at room temperature. Cyclopropanamine (20.4 mg, 0.358 mmol) was added. The mixture was stirred overnight at room temperature. After determining the reaction to be completed by LCMS, the reaction mixture was quenched with H₂O (10 mL) and extracted with EA (10 mL*2), The organic layer was separated and washed with brine (20 mL), And concentrated. The residue was purified by pre-HPLC to give the title compound. ¹H NMR (400 MHz, dmso) δ 12.97 (s, 1H), 8.91-8.74 (m, 2H), 8.65 (s, 1H), 8.16-8.04 (m, 2H), 8.00-7.91 (m, 1H), 7.72-7.54 (m, 2H), 3.58-3.49 (in, 1H), 3.43 (s, 1H), 3.14-3.07 (m, 1H), 2.96-2.88 (m, 1H), 2.17 (d, J=10.0 Hz, 1H), 2.05 (d, J=8.4 Hz, 1H), 1.97-1.72 (m, 4H), 1.72-1.53 (m, 2H), 1.38 (d, J=6.8 Hz, 3H), 0.75-0.63 (m, 4H). [M+1]⁺458.

### Example 22a and 22b: N-cyclopropyl-2-((S)-1-((1s,4R)-4-(6-fluoroquinolin-4-yl)cyclohexyl)ethyl)-1H-imidazo[4,5-c]pyridine-6-carboxamide and N-cyclopropyl-2-((R)-1-((1s,4S)-4-(6-fluoroquinolin-4-yl)cyclohexyl)ethyl)-1H-imidazo[4,5-c]pyridine-6-carboxamide

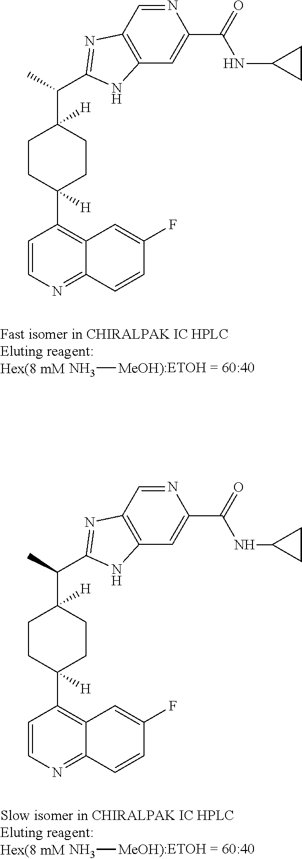

Each enantiomer of Example 22 (racemic 22a and 22b) was separated using preparative HPLC on a CHIRALPAK IC with Hex(8mMNH₃-MeOH):ETOH=60:40 as an eluent. The first one enantiomer eluted at the retention time of 2.515 min, 22a (12.77 mg), ¹H NMR (400 MHz, dmso) δ 12.96 (s, 1H), 8.90-8.70 (m, 2H), 8.68-8.54 (m, 1H), 8.20-8.05 (m, 2H), 7.98 (d, J=9.0 Hz, 1H), 7.67 (t, J=7.4 Hz, 1H), 7.59 (d, J=3.6 Hz, 1H), 3.61-3.37 (m, 2H), 2.92 (d, J=4.4 Hz, 1H), 2.18 (s, 1H), 2.05 (d, J=11.6 Hz, 1H), 1.95-1.72 (m, 4H), 1.72-1.54 (m, 2H), 1.38 (d, J=6.4 Hz, 3H), 1.15 (d, J=11.6 Hz, 1H), 0.75-0.63 (m, 4H). [M+1]⁺458. And the other enantiomer eluted at the retention time of 5.021 min, 22b (14.47 mg), ¹H NMR (400 MHz, dmso) δ 12.96 (s, 1H), 8.90-8.70 (m, 2H), 8.68-8.54 (m, 1H), 8.20-8.05 (m, 2H), 7.98 (d, J=9.0 Hz, 1H), 7.67 (t, J=7.4 Hz, 1H), 7.59 (d, J=3.6 Hz, 1H), 3.61-3.37 (m, 2H), 2.92 (d, J=4.4 Hz, 1H), 2.18 (s, 1H), 2.05 (d, J=11.6 Hz, 1H), 1.95-1.72 (m, 4H), 1.72-1.54 (m, 2H), 1.38 (d, J=6.4 Hz, 3H), 1.15 (d, J=11.6 Hz, 1H), 0.75-0.63 (m, 4H). [M+1]⁺458.

Example 22b can also be synthesized with other procedures:

### Example 22b: N-cyclopropyl-2-((R)-1-((1s,4s)-4-(6-fluoroquinolin-4-yl)cyclohexyl)ethyl)-3H-imidazo[4,5-c]pyridine-6-carboxamide

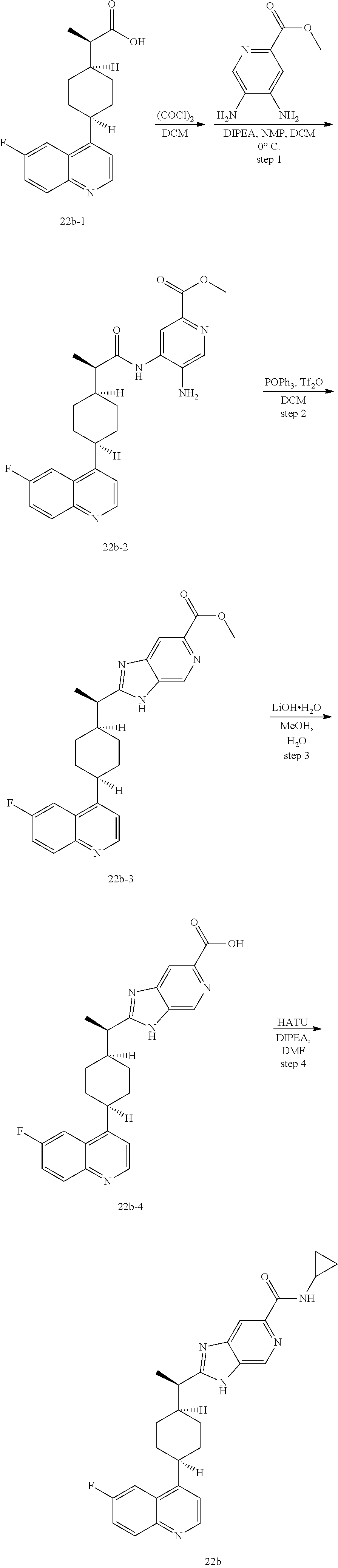

### Step 1: Methyl 5-amino-4-((R)-2-(1s,4S)-4-(6-fluoroquinolin-4-yl)cyclohexyl)propanamido)picolinate

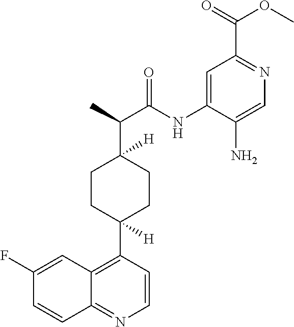 [SMILES: COC(=O)c1cc(NC(=O)[C@H](C)[C@H]2CC[C@@H](c3ccnc4ccc(F)cc43)CC2)c(N)cn1]

To a mixture of (R)-2-((1s,4S)-4-(6-fluoroquinolin-4-yl)cyclohexyl)propanoic acid (6.0 g, 20 mmol) and DMF (1 drop) in DCM (100 mL) was added oxalicdichloride (6.0 mL) dropwise, stirred for 1 hour at room temperature, the reaction solution was concentrated to give (R)-2-((1s,4S)-4-(6-fluoroquinolin-4-yl)cyclohexyl)propanoyl chloride. At 0° C., to a solution of methyl 4,5-diaminopicolinate (4.6 g, 27.5 mmol) and DIPEA (7.7 g, 60 mmol) in NMP (90 mL) was added the solution of (R)-2-((1s,4S)-4-(6-fluoroquinolin-4-yl)cyclohexyl)propanoyl chloride in DCM (65 mL) dropwise, the reaction mixture was stirred for 16 hours at room temperature, EA was added, and the mixture was washed with water and brine, dried over Na₂SO₄, concentrated and purified by sili-gel to give 6.1 g methyl 5-amino-4-((R)-2-((1s,4S)-4-(6-fluoroquinolin-4-yl)cyclohexyl)propanamido)picolinate.

### Step 2: Methyl 2-((R)-1-((1s,4S)-4-(6-fluoroquinolin-4-yl)cyclohexyl)ethyl)-3H-imidazo[4,5-c]pyridine-6-carboxylate

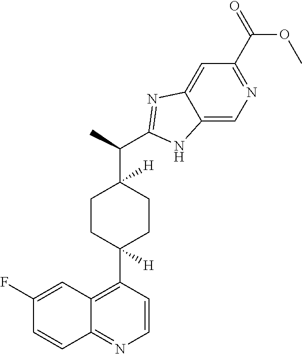 [SMILES: COC(=O)c1cc2nc([C@H](C)[C@H]3CC[C@@H](c4ccnc5ccc(F)cc54)CC3)[nH]c2cn1]

At 0° C., to a solution of POPh₃ (19 g, 68 mmol) in DCM (150 mL) was added a solution of Tf₂O (9.5 g, 33 mmol) in DCM (30 mL) dropwise, the reaction mixture was stirred for 15 min, then methyl 5-amino-4-((R)-2-((1s,4S)-4-(6-fluoroquinolin-4-yl)cyclohexyl)propanamido)picolinate (6.7 g, 14.9 mmol) in DCM (50 mL) was added in, the reaction mixture was stirred for 16 hours at room temperature, then warmed at room temperature for 16 hours. The reaction mixture was quenched with saturated NaHCO₃.aq, the DCM layer was concentrated and purified by sili-gel to give 5.6 g methyl 2-((R)-1-((1s,4S)-4-(6-fluoroquinolin-4-yl)cyclohexyl)ethyl)-3H-imidazo[4,5-c]pyridine-6-carboxylate.

### Step 3: 2-((R)-1-((1s,4S)-4-(6-fluoroquinolin-4 yl)cyclohexyl)ethyl)-3H-imidazo[4,5-c]pyridine-6-carboxylic Acid

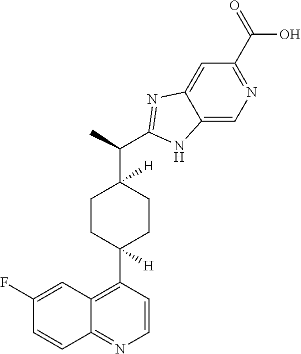 [SMILES: C[C@@H](c1nc2cc(C(=O)O)ncc2[nH]1)[C@H]1CC[C@@H](c2ccnc3ccc(F)cc32)CC1]

A solution of methyl 2-((R)-1-((1s,4S)-4-(6-fluoroquinolin-4-yl)cyclohexyl)ethyl)-3H-imidazo[4,5-c]pyridine-6-carboxylate (5.6 g, 12.9 mmol) and LiOH.H₂O (3.0 g, 71.4 mmol) was stirred for 16 hours at room temperature, the reaction mixture was concentrated, the residue's pH value was adjusted to 7 with 1N HCl.aq, the white solid was collected and dried in vacuo to give 4.1 g 2-((R)-1-((1s,4S)-4-(6-fluoroquinolin-4-yl)cyclohexyl)ethyl)-3H-imidazo[4,5-c]pyridine-6-carboxylic acid.

### Step 4: N-cyclopropyl-2-((R)-1-((1s,4S)-4-(6-fluoroquinolin-4 yl)cyclohexyl)ethyl)-3H-imidazo[4,5-c]pyridine-6-carboxamide

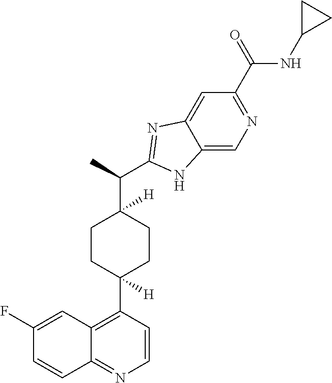 [SMILES: C[C@@H](c1nc2cc(C(=O)NC3CC3)ncc2[nH]1)[C@H]1CC[C@@H](c2ccnc3ccc(F)cc32)CC1]

A solution of 2-((R)-1-((1s,4S)-4-(6-fluoroquinolin-4-yl)cyclohexyl)ethyl)-3H-imidazo[4,5-c]pyridine-6-carboxylic acid (4.3 g, 10.3 mmol), cyclopropanamine (1.2 g, 21 mmol), HATU (4.5 g, 11.8 mmol) and DIPEA (6 mL, 35 mmol) in DMF (50 mL) was stirred for 16 hours at room temperature, the reaction mixture was poured into water (450 mL), the white solid was collected and purified by sili-gel, then recrystallized from MTBE/acetonitrile/n-hexane (40 mL/40 mL/40 mL) to give 1.7 g pure N-cyclopropyl-2-((R)-1-((1s,4S)-4-(6-fluoroquinolin-4-yl)cyclohexyl)ethyl)-3H-imidazo[4,5-c]pyridine-6-carboxamide. ¹H NMR (400 MHz, DMSO) δ_{H} 12.96 (s, 1H), 8.90-8.70 (m, 2H), 8.68-8.54 (m, 1H), 8.20-8.05 (m, 2H), 7.98 (d, J=9.0 Hz, 1H), 7.67 (t, J=7.4 Hz, 1H), 7.59 (d, J=3.6 Hz, 1H), 3.61-3.37 (m, 2H), 2.92 (d, J=4.4 Hz, 1H), 2.18 (s, 1H), 2.05 (d, J=11.6 Hz, 1H), 1.95-1.72 (m, 4H), 1.72-1.54 (m, 2H), 1.38 (d, J=6.4 Hz, 3H), 1.15 (d, J=11.6 Hz, 1H), 0.75-0.63 (m, 4H). [M+1]⁺458.

### Example 23: 2-(1-((1s,4s)-4-(6-fluoroquinolin-4-yl)cyclohexyl)ethyl)-N-methyl-1H-imidazo[4,5-c]pyridine-6-carboxamide

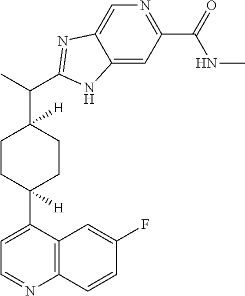 [SMILES: CNC(=O)c1cc2[nH]c(C(C)[C@H]3CC[C@@H](c4ccnc5ccc(F)cc54)CC3)nc2cn1]

¹H NMR (400 MHz, dmso) δ 12.96 (s, 1H), 8.95-8.78 (m, 2H), 8.71 (s, 1H), 8.22-8.04 (m, 2H), 7.99 (dd, J=11.2, 2.8 Hz, 1H), 7.72-7.55 (m, 2H), 3.51 (dd, J=10.8, 6.8 Hz, 1H), 3.43 (s, 1H), 2.84 (d, J=4.8 Hz, 3H), 2.17 (d, J=10.4 Hz, 1H), 2.06 (d, J=12.4 Hz, 1H), 1.97-1.72 (m, 4H), 1.72-1.53 (m, 2H), 1.38 (d, J=6.8 Hz, 3H). [M+1]432.

### Example 23a and 23b: 2-((S)-1-((1s,4R)-4-(6-fluoroquinolin-4-yl)cyclohexyl)ethyl)-N-methyl-1H-imidazo[4,5-c]pyridine-6-carboxamide and 2-((R)-1-((1s,4S)-4-(6-fluoroquinolin-4-yl)cyclohexyl)ethyl)-N-methyl-1H-imidazo[4,5-c]pyridine-6-carboxamide

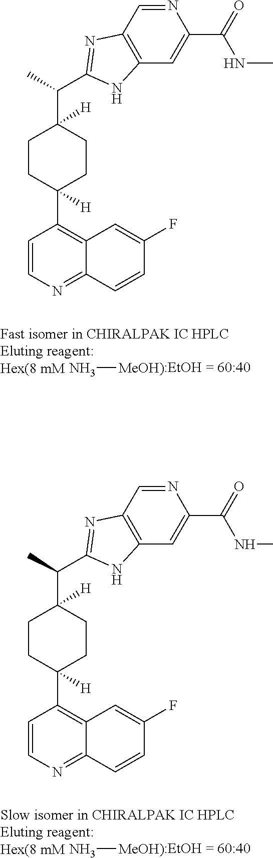

Each enantiomer of racemic compounds 23a and 23b was separated using preparative HPLC on a CHIRALPAK IC with Hex (8mMNH₃-MeOH):EtOH=60:40 as an eluent. The first one enantiomer was eluted at the retention time of 2.532 min, 23a (10.64 mg), ¹H NMR (400 MHz, dmso) δ 12.96 (s, 1H), 8.95-8.78 (m, 2H), 8.71 (s, 1H), 8.22-8.04 (m, 2H), 7.99 (dd, J=11.2, 2.8 Hz, 1H), 7.72-7.55 (m, 2H), 3.51 (dd, J=10.8, 6.8 Hz, 1H), 3.43 (s, 1H), 2.84 (d, J=4.8 Hz, 3H), 2.17 (d, J=10.4 Hz, 1H), 2.06 (d, J=12.4 Hz, 1H), 1.97-1.72 (m, 4H), 1.72-1.53 (m, 2H), 1.38 (d, J=6.8 Hz, 3H). [M+1]+432. And the other enantiomer was eluted at the retention time of 5.021 min, 23b (12.13 mg), ¹H NMR (400 MHz, dmso) δ 12.96 (s, 1H), 8.95-8.78 (m, 2H), 8.71 (s, 1H), 8.22-8.04 (m, 2H), 7.99 (dd, J=11.2, 2.8 Hz, 1H), 7.72-7.55 (m, 2H), 3.51 (dd, J=10.8, 6.8 Hz, 1H), 3.43 (s, 1H), 2.84 (d, J=4.8 Hz, 3H), 2.17 (d, J=10.4 Hz, 1H), 2.06 (d, J=12.4 Hz, 1H), 1.97-1.72 (m, 4H), 1.72-1.53 (m, 2H), 1.38 (d, J=6.8 Hz, 3H). [M+1]⁺432.

### Example 24: N-cyclopropyl-2-((R)-1-((1s,4S)-4-(6-fluoroquinolin-4-yl)cyclohexyl)ethyl)-3H-imidazo[4,5-b]pyridine-6-carboxamide

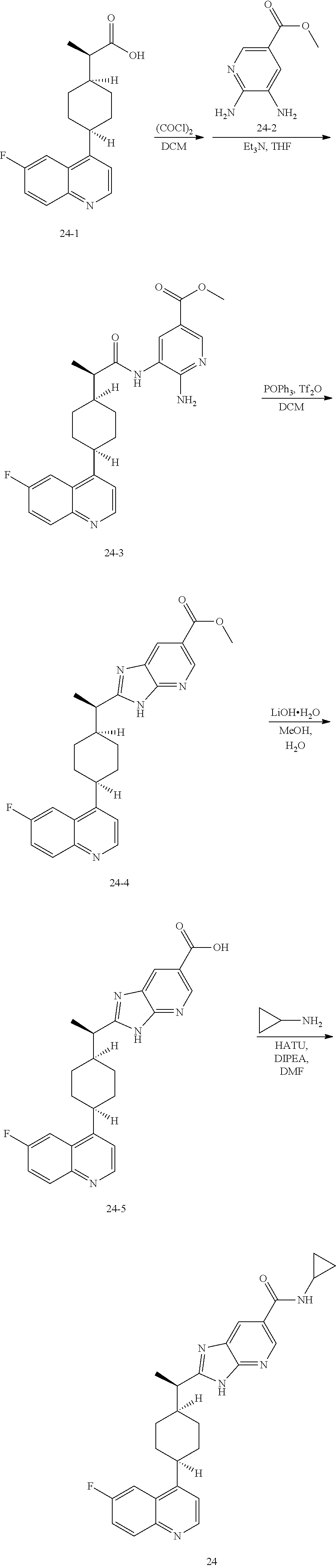

### Step 1: Methyl 6-amino-5-((R)-2-((1s,4S)-4-(6-fluoroquinolin-4-yl)cyclohexyl)propanamido)nicotinate

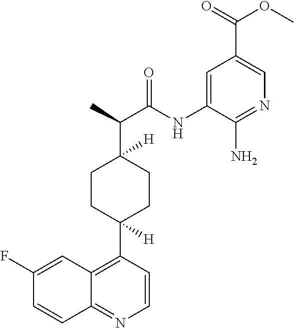 [SMILES: COC(=O)c1cnc(N)c(NC(=O)[C@H](C)[C@H]2CC[C@@H](c3ccnc4ccc(F)cc43)CC2)c1]

To a solution of (R)-2-((1s,4S)-4-(6-fluoroquinolin-4-yl)cyclohexyl)propanoic acid (400 mg, 1.33 mmol) in DCM (10 mL) were added DMF (1 drop) and oxalyl chloride (200 mg, 1.55 mmol) dropwise, the mixture was stirred for 2 hours at room temperature, concentrated to give (R)-2-((1s,4S)-4-(6-fluoroquinolin-4-yl)cyclohexyl)propanoyl chloride. At 0° C., to a solution of methyl 5,6-diaminonicotinate (700 mg, 4.2 mmol) and Et₃N (700 mg, 7.0 mmol) in THF (20 mL) was added the solution of (R)-2-((1s,4S)-4-(6-fluoroquinolin-4-yl)cyclohexyl)propanoyl chloride in THF (5 mL) dropwise, the reaction mixture was stirred for 30 min at 0° C., concentrated, EA added in, washed with water and brine, dried over Na₂SO₄, concentrated and purified by sili-gel to give methyl 6-amino-5-((R)-2-((1s,4S)-4-(6-fluoroquinolin-4-yl)cyclohexyl)propanamido)-nicotinate.

### Step 2: Methyl 2-((R)-1-((1s,4S)-4-(6-fluoroquinolin-4 yl)cyclohexyl)ethyl)-3H-imidazo[4,5-b]pyridine-6-carboxylate

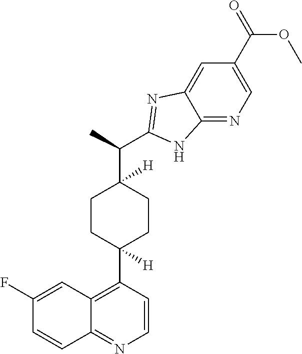 [SMILES: COC(=O)c1cnc2[nH]c([C@H](C)[C@H]3CC[C@@H](c4ccnc5ccc(F)cc54)CC3)nc2c1]

At 0° C., to a solution of POPh₃ (560 mg, 2.0 mmol) in DCM (20 mL) was added a solution of Tf₂O (280 mg, 1.0 mmol) in DCM (5 mL) dropwise, the reaction mixture was stirred for 15 min, then methyl 6-amino-5-((R)-2-((1s,4S)-4-(6-fluoroquinolin-4-yl)cyclohexyl)propanamido)nicotinate (400 mg, 0.89 mmol) in DCM (5 mL) was added in, the reaction mixture was stirred for 30 min at 0° C., then warmed at room temperature for 16 hours. The reaction mixture was quenched with saturated NaHCO₃.aq, the DCM layer was concentrated and purified by sili-gel to give methyl 2-((R)-1-((1s,4S)-4-(6-fluoroquinolin-4-yl)cyclohexyl)ethyl)-3H-imidazo[4,5-b]pyridine-6-carboxylate.

### Step 3: 2-((R)-1-((1s,4S)-4-(6-fluoroquinolin-4-yl)cyclohexyl)ethyl)-3H-imidazo[4,5-b]pyridine-6-carboxylic Acid

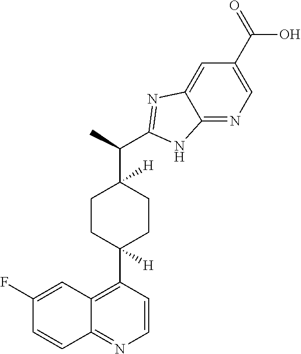 [SMILES: C[C@@H](c1nc2cc(C(=O)O)cnc2[nH]1)[C@H]1CC[C@@H](c2ccnc3ccc(F)cc32)CC1]

A solution of methyl 2-((R)-1-((1s,4S)-4-(6-fluoroquinolin-4-yl)cyclohexyl)ethyl)-3H-imidazo[4,5-b]pyridine-6-carboxylate (160 mg, 0.37 mmol) and LiOH.H₂O (40 mg, 1.0 mmol) was stirred for 48 hours at room temperature, the reaction mixture was concentrated, the residue's pH value was adjusted to 5 with 1N HCl.aq, the white solid was collected and dried in vacuo to give 120 mg 2-((R)-1-((1s,4S)-4-(6-fluoroquinolin-4-yl)cyclohexyl)ethyl)-3H-imidazo[4,5-b]pyridine-6-carboxylic acid.

### Step 4: N-cyclopropyl-2-((R)-1-((1s,4S)-4-(6-fluoroquinolin-4-yl)cyclohexyl)ethyl)-3H-imidazo[4,5-b]pyridine-6-carboxamide

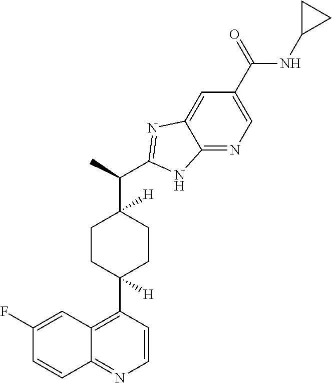 [SMILES: C[C@@H](c1nc2cc(C(=O)NC3CC3)cnc2[nH]1)[C@H]1CC[C@@H](c2ccnc3ccc(F)cc32)CC1]

A solution of 2-((R)-1-((1s,4S)-4-(6-fluoroquinolin-4-yl)cyclohexyl)ethyl)-3H-imidazo[4,5-b]pyridine-6-carboxylic acid (120 mg, 0.29 mmol), cyclopropanamine (40 mg, 0.7 mmol), HATU (140 mg, 0.37 mmol) and DIPEA (150 mg, 1.16 mmol) in DMF (8 mL) was stirred for 16 hours at room temperature, EA was added in, the mixture was washed with water and brine, dried over Na₂SO₄, concentrated and purified by sili-gel to give N-cyclopropyl-2-((R)-1-((1s,4S)-4-(6-fluoroquinolin-4-yl)cyclohexyl)ethyl)-3H-imidazo[4,5-b]pyridine-6-carboxamide. ¹H NMR (400 MHz, DMSO-d) δ_{H} 13.0 (m, 1H), 8.87 (d, J=4.4 Hz, 1H), 8.73 (s, 1H), 8.51 (s, 1H), 8.27 (s, 1H), 8.10 (dd, J=9.2, 5.6 Hz, 1H), 7.98 (dd, J=6.8, 2.4 Hz, 1H), 7.66 (dt, J=8.8, 2.4 Hz, 1H), 7.58 (d, J=4.4 Hz, 1H), 3.43 (m, 2H), 2.87 (m, 1H), 1.55-2.19 (m, 9H), 1.37 (d, J=6.8 Hz, 3H), 1.13-1.26 (m, 2H), 0.71 (m, 2H), 0.59 (m, 2H).

### Example 25: 2-(1-((1s,4s)-4-(6-fluoroquinolin-4-yl)cyclohexyl)ethyl)-3,7-dihydro-8H-imidazo[4,5-g]quinazolin-8-one

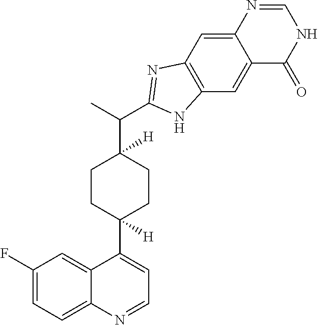 [SMILES: CC(c1nc2cc3nc[nH]c(=O)c3cc2[nH]1)[C@H]1CC[C@@H](c2ccnc3ccc(F)cc32)CC1]

To a solution of (R)-2-((1s,4S)-4-(6-fluoroquinolin-4-yl)cyclohexyl)propanoic acid (301 mg, 1.0 mmol) in PPA (5 mL) was added 6,7-diaminoquinazolin-4(3H)-one (200 mg, 1.15 mmol). The mixture was stirred at 120° C. for 18 hours. The reaction mixture was poured into water (50 ml), and sonicated for 5 mins, filtered and washed with water (5 ml*2). The crude solid was purified by column chromatography on silica gel (DCM/MeOH=100/0 to 90/10) to give the title compound. ¹H NMR (400 MHz, DMSO-d₆) δ_{H} 12.68 (m, 1H), 11.95 (s, 1H), 8.87 (d, J=4.4 Hz, 1H), 8.15-8.26 (m, 1H), 8.10 (dd, J=9.2, 6.0 Hz, 1H), 7.98 (m, 2H), 7.60-7.79 (m, 2H), 7.60 (d, J=4.4 Hz, 1H), 3.47 (m, 2H), 2.19 (m, 1H), 2.06 (m, 1H), 1.86 (m, 3H), 1.78 (m, 1H), 1.67 (m, 1H), 1.58 (m, 1H), 1.38 (m, 3H), 1.19 (m, 1H). [M+H]+=442.1.

### Example 26: N-ethyl-2-((R)-1-((1s,4S)-4-(6-fluoroquinolin-4-yl)cyclohexyl)ethyl)-3H-imidazo[4,5-c]pyridine-6-carboxamide

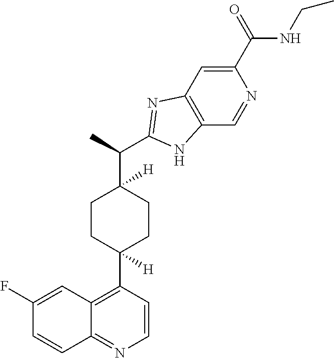 [SMILES: CCNC(=O)c1cc2nc([C@H](C)[C@H]3CC[C@@H](c4ccnc5ccc(F)cc54)CC3)[nH]c2cn1]

A solution of 2-((R)-1-((1s,4S)-4-(6-fluoroquinolin-4-yl)cyclohexyl)ethyl)-3H-imidazo[4,5-c]pyridine-6-carboxylic acid (400 mg, 0.96 mmol), Ethylamine Hydrochloride (200 mg, 2.44 mmol), HATU (600 mg, 1.58 mmol) and Et₃N (700 mg, 7.0 mmol) in DMF (10 mL) was stirred for 16 hours at room temperature, EA was added in, and the solution was washed with water and brine, dried over Na₂SO₄, concentrated and purified by sili-gel to give 145 mg title product. ¹H NMR (400 MHz, DMSO-d) δ_{H} 12.95 (s, 1H), 8.87-8.66 (m, 3H), 8.20-8.05 (m, 2H), 7.98 (dd, J=11.0, 2.6 Hz, 1H), 7.70-7.63 (m, 1H), 7.59 (d, J=4.3 Hz, 1H), 3.47 (m, 2H), 2.18 (m, 1H), 2.04 (m, 1H), 1.95-1.53 (m, 6H), 1.38 (d, J=6.7 Hz, 3H), 1.14 (t, J=7.1 Hz, 4H).

### Example 27a and 27b (Comparative Example 1): 4-((1R,4s)-4-((S)-1-(5,6-difluoro-1H-benzo[d]imidazol-2-yl)ethyl)cyclohexyl)-6-fluoroquinoline and 4-((1S,4s)-4-((R)-1-(5,6-difluoro-1H-benzo[d]imidazol-2-yl)ethyl)cyclohexyl)-6-fluoroquinoline

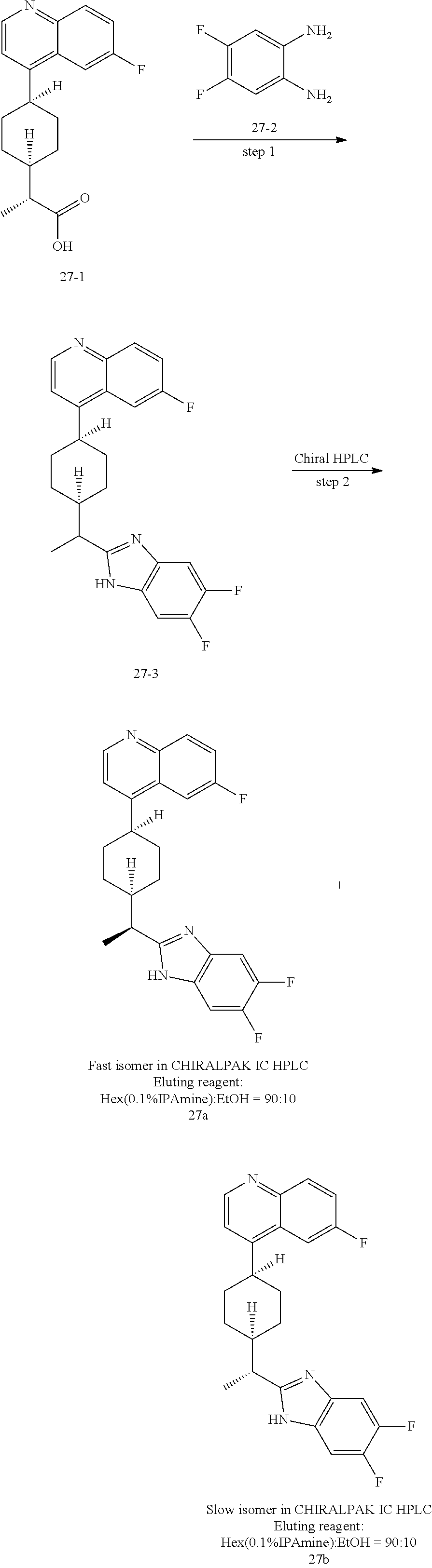

### Step 1: 4-((1s,4s)-4-(1-(5,6-difluoro-1H-benzo[d]imidazol-2-yl)ethyl)cyclohexyl)-6-fluoroquinoline

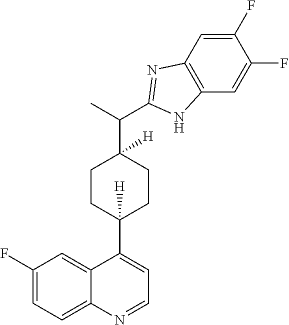

To a solution of (R)-2-((1s,4S)-4-(6-fluoroquinolin-4-yl)cyclohexyl)propanoic acid (0.2 g, 0.66 mmol) in DMF (10 mL), HATU (0.3 g, 0.8 mmol), DIEA (0.5 mL) were added at room temperature. Then 4,5-difluorobenzene-1,2-diamine (0.14 g, 0.8 mmol) was added. The mixture was stirred at 20-30° C. for 48 hours. The reaction mixture was then quenched with H₂O (50 mL) and extracted with EA (50 mL), The organic layer was separated and washed with brine (100 mL) and concentrated to give the crude product (R)-N-(2-amino-4,5-difluorophenyl)-2-((1s,4S)-4-(6-fluoroquinolin-4-yl)cyclohexyl)propanamide, which was used in the next step without further purification.

A solution of (R)-N-(2-amino-4,5-difluorophenyl)-2-((1s,4S)-4-(6-fluoroquinolin-4-yl)cyclohexyl)propanamide in HOAc (20 mL) was stirred at 100° C. for 18 hours. The solvent was evaporated. The crude residue was dissolved with EA (50 mL) and washed with saturated NaHCO₃ solution (50 mL). The organic phase was separated and purified by pre-HPLC to give 4-((1s,4s)-4-(1-(5,6-difluoro-1H-benzo[d]imidazol-2-yl)ethyl)cyclohexyl)-6-fluoroquinoline. ¹H NMR (400 MHz, DMSO-d) δ_{H} 12.45 (s, 1H), 8.86 (d, J=4.4 Hz, 1H), 8.09 (dd, J=9.2, 6.0 Hz, 1H), 7.98 (dd, J=10.8, 2.4 Hz, 1H), 7.71-7.62 (m, 1H), 7.58-7.40 (m, 3H), 3.45-3.38 (m, 2H), 2.16-1.99 (m, 2H), 1.94-1.46 (m, 7H), 1.33 (d, J=6.8 Hz, 3H). [M+H]⁺=409.9.

### Step 2: 4-((1R,4s)-4-((S)-1-(5,6-difluoro-1H-benzo[d]imidazol-2-yl)ethyl)cyclohexyl)-6-fluoroquinoline and 4-((1S,4s)-4-((R)-1-(5,6-difluoro-1H-benzo[d]imidazol-2-yl)ethyl)cyclohexyl)-6-fluoroquinoline

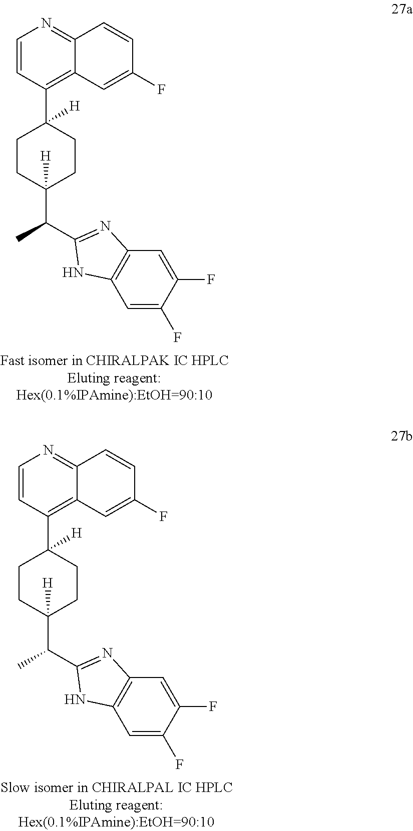

Each enantiomer of racemic 27a and 27b was separated using preparative HPLC on a CHIRALPAK IC with Hex:EtOH=90:10 as an eluent. The first one enantiomer was eluted at the retention time of 2.001 min, and the other enantiomer was eluted at the retention time of 2.328 min. The eluant of 27a was concentrated to give desired product as white solid (4.20 mg). ¹H NMR (MeOH-d₆) δ_{H} 8.78 (d, J=4.8 Hz, 1H), 8.07 (dd, J=9.2, 5.6 Hz, 1H), 7.86 (dd, J=10.4, 2.8 Hz, 1H), 7.68-7.48 (m, 2H), 7.37 (br, 2H), 3.47-3.41 (m, 2H), 2.27-2.14 (m, 2H), 2.06-1.63 (m, 7H), 1.42 (d, J=6.4 Hz, 3H). [M+H]⁺ 09.9. The eluant of 27b was concentrated 27b to give desired product as white solid (60.3 mg). ¹H NMR (MeOH-d₆) δ_{H} 8.69 (d, J=4.8 Hz, 1H), 7.98 (dd, J=9.2, 5.6 Hz, 1H), 7.77 (dd, J=10.8, 2.4 Hz, 1H), 7.61-7.45 (m, 2H), 7.27 (br, 2H), 3.38-3.33 (m, 2H), 2.08 (br, 2H), 1.99-1.52 (m, 7H), 1.33 (d, J=6.8 Hz, 3H). [M+H]⁺=409.9.

Examples 28a to example 29 were synthesized with similar procedure with example 27a and 27b.

### Example 28a and 28b (Comparative Example 2): methyl 2-((S)-1-((1s,4R)-4-(6-fluoroquinolin-4-yl)cyclohexyl)ethyl)-1H-benzo[d]imidazole-5-carboxylate and methyl 2-((R)-1-((1s,4S)-4-(6-fluoroquinolin-4-yl)cyclohexyl)ethyl)-1H-benzo[d]imidazole-5-carboxylate

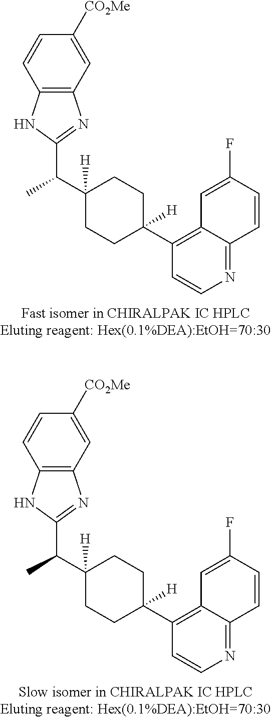 [SMILES: COC(=O)c1ccc2[nH]c([C@@H](C)[C@H]3CC[C@@H](c4ccnc5ccc(F)cc54)CC3)nc2c1 ; COC(=O)c1ccc2[nH]c([C@H](C)[C@H]3CC[C@@H](c4ccnc5ccc(F)cc54)CC3)nc2c1]

Each enantiomer of racemic 28a and 28b was separated using preparative HPLC on a CHIRALPAK IC with Hex:EtOH=70:30 as an eluent. The first one enantiomer was eluted at the retention time of 1.534 min, 28a (7.65 mg). ¹H NMR (MeOH-d₆) δ_{H} 8.68 (d, J=4.8 Hz, 1H), 8.13 (s, 1H), 7.97 (dd, J=9.2, 5.6 Hz, 1H), 7.83-7.75 (m, 2H), 7.53-7.42 (m, 3H), 3.82 (s, 3H), 3.45-3.31 (m, 2H), 2.15-2.07 (m, 2H), 1.95-1.54 (m, 7H), 1.36 (d, J=6.8 Hz, 3H). [M+H]+=431.8. And the other enantiomer was eluted at the retention time of 2.048 min, 28b: (37.58 mg). ¹H NMR (MeOH-d₆) δ_{H} 8.77 (d, J=4.4 Hz, 1H), 8.26-8.05 (m, 2H), 7.94-7.81 (m, 2H), 7.61-7.55 (m, 3H), 3.91 (s, 3H), 3.55-3.37 (m, 2H), 2.28-2.13 (m, 2H), 2.01-1.68 (m, 7H), 1.45 (d, J=6.8 Hz, 3H). [M+H]+=431.8.

### Example 29 (Comparative Example 2): 6-fluoro-4-((1S,4s)-4-((R)-1-(4,5,6-trifluoro-1H-benzo[d]imidazol-2-yl)ethyl)cyclohexyl)quinoline

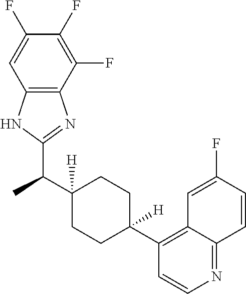 [SMILES: C[C@@H](c1nc2c(F)c(F)c(F)cc2[nH]1)[C@H]1CC[C@@H](c2ccnc3ccc(F)cc32)CC1]

¹H NMR (MeOH-d₆) δ_{H} 12.78-13.07 (m, 1H), 8.87 (d, J=4.0 Hz, 1H), 8.10-7.97 (m, 2H), 7.68-7.40 (m, 3H), 3.43-3.42 (m, 2H), 2.15-2.02 (m, 2H), 1.89-1.56 (m, 6H), 1.35 (d, J=6.4 Hz, 3H), 1.17-1.14 (m, 1H).

### Example F: Biological Assays

### HeLa Cell-Based IDO1 Kyn (Kynurenine) Production Assay:

The inhibitory activity of IDO1 inhibitors is determined by using a colorimetric reaction to measure Kyn generated from L-Trp (L-Tryptophon) oxidation by cellular IDO1 in HeLa cells after induction of IDO1 expression by IFN-γ. Hela cells were obtained from the American Type Culture Collection and recovered in 10% FBS-containing phenol red-free DMEM medium. Cells were plated onto a 96-well plate (100 μl/well) at 8000 cells per well and kept at 37° C. in a humidified incubator supplied with 5% CO₂. 4 hours later, Human recombinant IFN-γ (8901SC, CST) was added to cells (final concentration 100 ng/mL) to stimulate endogenous IDO1. Compounds at different concentrations diluted in dimethylsulfoxide (DMSO) were added simultaneously with IFN-γ and 0.4 mM L-Trp. Cells were kept at 37° C. in a humidified incubator supplied with 5% CO₂. After 48 hours of incubation, 100 μl supernatant from each well was removed to a new plate. The protein in the medium was precipitated with the addition of 8 μl 6N trichloroacetic acid. The plate was incubated at 60° C. for 30 minutes and then centrifugation at 2500 rpm for 10 minutes to remove sediments. 80 μl supernatants were carefully removed to a new clean plate and added with an equal volume of 2% 4-(Dimethylamino)benzaldehyde (D2004, Sigma) dissolved in glacial acetic acid. The absorbance at 480 nm wavelength derived from Kyn was measured using a PHERAstar FS plate reader (BMG LABTECH). The IC₅₀ for each compound was derived from fitting the dose-response data to the four-parameter logistic model by using XLfit software (IDBS).

### Plasma Protein Binding Assay:

To evaluate the plasma protein binding extent, the bound (fb) and unbound (fu) fractions of test compound will be determined in vitro by equilibrium dialysis approach, using a 96-well dialysis device (HT Dialysis, Gales Ferry, Conn., USA). The equilibrium dialysis will be conducted in duplicate. A hundred and fifty μL of 50% plasma spiked with the test compound (final concentration of 5 μM) will be added into the donor side and 150 μL of Phosphate Buffer (PB) (0.002% Tween-80) into the corresponding receiving side. The device will be then sealed with adhesive film and shook at 80 rpm in the water bath at 37° C. for 6 h. At the end of incubation, 10 μL plasma sample will be transferred from the donor side into a 1.5 mL microcentrifuge tube, added with 90 μL PB (0.002% Tween-80), vortexed well and proteins precipitated by acetonitrile containing internal standard (IS). Ninety μL of PB (0.002% Tween-80) sample will be transferred from the receiver side into a 1.5 mL microcentrifuge tube, added with 10 μL 50% blank plasma, vortexed well and proteins precipitated by acetonitrile containing IS.

For recovery check, 10 of the donor side loading samples will be aliquoted into a 1.5 mL microcentrifuge tube (in duplicate), added with 90 μL PB (0.002% Tween-80), vortexed well and proteins precipitated by acetonitrile containing IS.

For plasma stability check, 10 μL of the donor side loading samples will be aliquoted into a 1.5 mL microcentrifuge tube (in duplicate), incubated in the water bath at 37° C. for 6 h, added with 90 μL PB (0.002% Tween-80) solution at the end of the incubation, vortexed well and proteins precipitated by acetonitrile containing IS.

Bioanalysis

Appropriate LC-MS/MS methods will be developed for the analysis of the test or control compounds in the incubates.

Data Analysis

The unbound fraction (fu) of the test compound and positive control compounds in 50% plasma will be calculated using the following equations.

$${f}_{u , diluted \text{-} plasma} = \frac{{C}_{R}}{{C}_{D}} \times 100 \%$$

$${f}_{u} = \frac{1}{( \frac{1}{{f}_{u , diluted \text{-} plasma}} - 1 ) * D + 1}$$

Where C_{R} is the area ratio of the test compounds to the IS in the receiving side, C_{D} is the area ratio of the test compound to the IS in the corresponding donor side and D is the dilution factor of plasma.

TABLE 1
Cellular activity data EC₅₀s (Hela Cell-Based IDO1
and Plasma Protein Binding) of 1H-benzo[d]imidazol

| | Cell-Based EC₅₀ (nM) | Plasma Protein Binding assay (fu %) |
|---|---|---|
| Ex. No. | Hela IDO1 | Human |
| 1 | 64.1 | |
| 2 | 50.6 | |
| 3 | 74.5 | |
| 4 | 26.9 | 0.20 |
| 5 | 38.8 | |
| 6 | 40.2 | |
| 7 | 470.3 | |
| 8 | 46.1 | |
| 9 | 12.6 | |
| 10 | 27.1 | |
| 10a | 787.9 | |
| 10b | 17.1 | |
| 11 | 84.1 | |
| 12 | 958.9 | |
| 13 | 62.9 | |
| 14 | 14.4 | |
| 15 | 86.7 | |
| 16 | >1000 | |
| 17 | >1000 | |
| 18 | 122.3 | |
| 19 | 193.2 | |
| 20 | 6.9 | |
| 21 | 8.9 | |
| 22 | 3.12 | |
| 22a | 266.7 | 0.56 |
| 22b | 0.96 | 0.47 |
| 23 | 7.12 | |
| 23a | 223.4 | 0.72 |
| 23b | 3.83 | 0.53 |
| 24 | 24.9 | |
| 25 | 82.4 | |
| 26 | 0.77 | |
| 27b | 0.42 | 0.034 |
| 28b | 0.55 | 0.067 |
| 29 | 0.85 | 0.02 |

This table data show non-amide-substituted imidazo compounds have higher PPB than amide-substituted compounds.

The representative compounds disclosed herein exhibited of inhibiting Hela Cell-Based IDO1 with EC₅₀ values ranging less than 10000 nM.

It is to be understood that, if any prior art publication is referred to herein; such reference does not constitute an admission that the publication forms a part of the common general knowledge in the art in any country.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and Examples should not be construed as limiting the scope of the invention.

1. A compound of Formula (I): 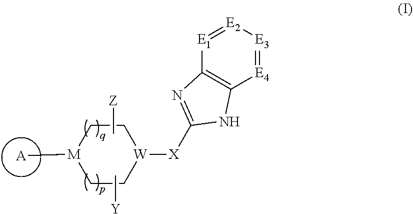
or a stereoisomer thereof, or a pharmaceutically acceptable salt thereof,
wherein:
M is CH or N;
W is CH or N;
p is 1, 2 or 3;
q is 0, 1 or 2;
X is —CR⁵R⁶—, —CHR⁵CHR⁶— or a single bond;
R⁵ and R⁶ are each independently hydrogen, halogen, cyano, C₁₋₄alkyl, C₁₋₄ haloalkyl, C₁₋₄alkoxy, or C₃₋₆cycloalkyl; or (R⁵ and R⁶), and/or (R⁵ and Y), together with the atom(s) to which they are attached, form a fused C₃₋₈cycloalkyl ring, and said ring is optionally substituted with halogen, C₁₋₄ haloalkyl and C₁₋₄ alkyl;
Y and Z are each independently selected from hydrogen, halogen, C₁₋₈ alkyl, C₂₋₈ alkenyl, C₂₋₈ alkynyl, C₁₋₈ haloalkyl, C₃₋₈ cycloalkyl, heterocyclyl, aryl, or heteroaryl; or Z and Y, together with the atom(s) to which they are attached, form a bridged cyclic or heterocyclic ring optionally substituted with a substituent selected from halogen, C₁₋₄ haloalkyl, C₁₋₄ alkyl and C₁₋₄alkoxy;
Ring A is a monocyclic or bicyclic aromatic hydrocarbon ring or a monocyclic or bicyclic aromatic heterocyclic ring, each having 5- to 10-ring members; and Ring A is optionally substituted with at least one substituent R⁷ as long as valence and stability permit;
E₁, E₂, E₃ and E₄ are each independently selected from CR³ or N;
R³ is each independently selected from hydrogen, halogen, cyano, C₁₋₈ alkyl, C₃₋₈cycloalkyl, C₂₋₈ alkenyl, C₂₋₈ alkynyl, C₁₋₈ haloalkyl, heterocyclyl, aryl, heteroaryl, —C(O)NR¹R², nitro, —C(O)OR¹, —C(O)R¹, —OR¹, —SR¹, —NR¹R², —SO₂R¹, —SO₂NR¹R², —SOR¹, —NR¹SO₂R², —NR¹SOR², —NR¹C(O)OR² or —NR¹C(O)R², wherein said C₁₋₈ alkyl, C₂₋₈ alkenyl, C₂₋₈ alkynyl, C₁₋₈ haloalkyl, C₃₋₈ cycloalkyl, heterocyclyl, aryl, and heteroaryl are each independently optionally substituted with 1 or 2 substituents R¹⁰;
R¹ and R² are each independently H, C₁₋₈ alkyl, C₃₋₈ cycloalkyl, heterocyclyl, aryl, or heteroaryl, wherein said C₁₋₈ alkyl, C₃₋₈ cycloalkyl, heterocyclyl, aryl, and heteroaryl are each independently optionally substituted with 1 or 2 substituents R¹⁰, or R¹ and R², together with the nitrogen atom to which they are attached, form a ring comprising 0, 1, 2, 3 or 4 additional heteroatoms selected from —NH, —O—, —S—, —SO— or —SO₂—, and said ring is optionally substituted with at least one substituent R¹⁰;
provided that at least one of E₁, E₂, E₃ and E₄ is CR³, wherein R³ is —C(O)NR¹R², wherein R¹ and R² are defined above;
alternatively, two adjacent R³, if present, together with the atom(s) to which they are attached, form a lactam ring, said ring comprising, in addition to the nitrogen atom forming the lactam ring, 0, 1 or 2 additional heteroatoms independently selected from nitrogen, oxygen or sulfur, and said ring is optionally substituted with halogen, C₁₋₄ haloalkyl and C₁₋₄ alkyl;
R⁷ is independently selected from hydrogen, halogen, C₁₋₈ alkyl, C₂₋₈ alkenyl, C₂₋₈ alkynyl, C₁₋₄ haloalkyl, C₃₋₈ cycloalkyl, heterocyclyl, aryl, or heteroaryl, wherein said C₁₋₄ alkyl, C₂₋₈ alkenyl, C₂₋₈ alkynyl, C₁₋₈ haloalkyl, C₃₋₈ cycloalkyl, heterocyclyl, aryl, and heteroaryl are each independently optionally substituted with 1 or 2 substituents R¹⁰;
R¹⁰, at each occurrence, is independently hydrogen, halogen, C₁₋₈ haloalkyl, C₁₋₈ alkyl, C₂₋₈ alkenyl, C₂₋₈ alkynyl, C₃₋₈ cycloalkyl, aryl, heteroaryl, heterocyclyl, oxo, —C₁₋₄ alkyl-NR^{a}R^{b}, —CN, —OR¹, —NR^{a}R^{b}, —C(O)R^{a}, —C(O)OR^{a}, —CONR^{a}R^{b}, —C(═NR^{a})NR^{b}R^{c}, nitro, —NR^{a}C(O)R^{b}, —NR^{a}C(O)NR^{a}R^{b}, —NR^{a}C(O)OR^{b}, —SO₂R^{a}, —NR^{a}SO₂NR^{b}R^{c}, —NR^{a}SOR^{b} or —NR^{a}SO₂R^{b}, wherein said C₁₋₈ alkyl, C₁₋₈ haloalkyl, C₃₋₈ cycloalkyl, aryl, heteroaryl, or heterocyclyl group are each independently optionally substituted by one, two or three substituents selected from halo, hydroxyl, C₁₋₄ alkyl, C₁₋₄ alkyloxy, C₁₋₄ haloalkyl, and C₁₋₄ haloalkyloxy, wherein R^{a}, R^{b}, and R^{c} are each independently selected from H, C₁₋₄ haloalkyl, C₁₋₄ alkyl, C₂₋₄ alkenyl, C₂₋₄ alkynyl, C₃₋₆ cycloalkyl, heterocyclyl, aryl, and heteroaryl, each of which is optionally substituted by one or more halogen, C₁₋₄ haloalkyl and C₁₋₄ alkyl, or (R^{a} and R^{b}), and/or (R^{b} and R^{c}) together with the atom(s) to which they are attached, form a ring selected from a heterocyclyl or heteroaryl ring optionally substituted by halogen, C₁₋₄ haloalkyl or C₁₋₄ alkyl.

2. The compound of claim 1, wherein E₁, E₂, E₃ and E₄ are defined as follows:
(a) E₁ is CR³, E₂ is —CC(O)NR¹R², E₃ is CR³, and E₄ is CR³;
(b) E₁ is CR³, E₂ is CR³, E₃ is —CC(O)NR¹R², and E₄ is CR³;
(c) E₁ is N, E₂ is —CC(O)NR¹R², E₃ is CR³, and E₄ is CR³;
(d) E₁ is N, E₂ is CR³, E₃ is —CC(O)NR¹R², and E₄ is CR³;
(e) E₁ is CR³, E₂ is —CC(O)NR¹R², E₃ is CR³, and E₄ is N;
(f) E₁ is CR³, E₂ is CR³, E₃ is —CC(O)NR¹R², and E₄ is N;
(g) E₁ is N, E₂ is —CC(O)NR¹R², E₃ is CR³, and E₄ is N;
(h) E₁ is N, E₂ is CR³, E₃ is —CC(O)NR¹R², and E₄ is N;
(i) E₁ is CR³, E₂ is —CC(O)NR¹R², E₃ is N, and E₄ is CR³;
(j) E₁ is CR³, E₂ is N, E₃ is —CC(O)NR¹R², and E₄ is CR³;
(k) E₁ is N, E₂ is —CC(O)NR¹R², E₃ is N, and E₄ is CR³;
(l) E₁ is CR³, E₂ is N, E₃ is —CC(O)NR¹R², and E₄ is N;
wherein R¹, R², and R³ are defined as for formula (I).

3. The compound of claim 1 or 2, wherein p is 1, and q is 1.

4. The compound of claim 1 or 2, wherein p is 1 and q is 0.

5. The compound of claim 1 or 2, wherein W is N and M is CH, or W and M are both N, or W and M are both CH, or W is CH and M is N.

6. The compound of claim 1 or 2, wherein the 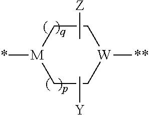
moiety is 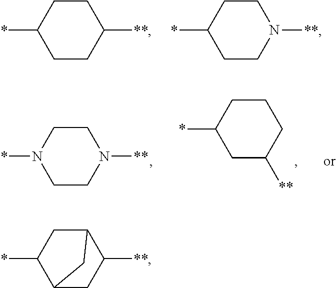
wherein * indicates a link to the ring A, and ** indicates a link to X.

7. The compound of claim 1 or 2, wherein X is —CR⁵R⁶—, wherein R⁵ is C₁₋₄alkyl, C₁₋₄haloalkyl, C₁₋₄alkoxy, or C₃₋₆cycloalkyl, and R⁶ is hydrogen.

8. The compound of claim 1 or 2, wherein X is —CR⁵R⁶—, wherein R⁵ is C₁₋₄alkyl, C₁₋₄haloalkyl, C₁₋₄alkoxy, or C₃₋₆cycloalkyl, and R⁶ is hydrogen, and the 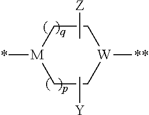
moiety is 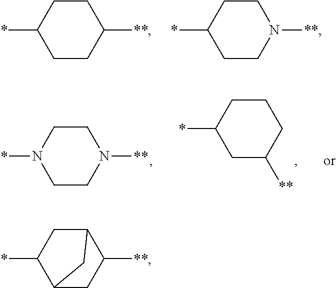
wherein * indicates a link to the ring A, and ** indicates a link to X.

9. The compound of claim 2, wherein the compound of Formula (I) is a compound of Formula (Ia): 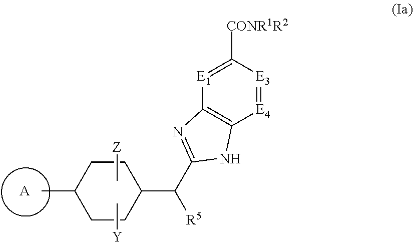
wherein the variables R¹, R², R⁵, Z, Y, E₁, E₃, E₄ and A are defined as for Formula (I).

10. The compound of claim 1 or 2, wherein Z and Y, together with the atoms to which they are attached, form a bridged bicyclic ring optionally substituted with a substituent selected from halogen, C₁₋₄haloalkyl, C₁₋₄ alkyl and C₁₋₄alkoxy; Preferably, Z and Y, together with the atoms to which they are attached, form a bridged bicyclic ring selected from bicyclo[2.2.1]heptyl (e.g., bicyclo[2.2.1]heptan-2-yl), born-2-yl, bicyclo[2.2.2]octyl, bicyclo[3.2.1]octyl, bicyclo[3.3.1]nonyl, or bicyclo[3.3.2.]decyl. More preferably, the bridged bicyclic ring is bicyclo[2.2.1]heptyl or bicyclo[2.2.2]octyl.

11. The compound of any one of claim 1 or 2 or 9, wherein is selected from Formula (Ib): 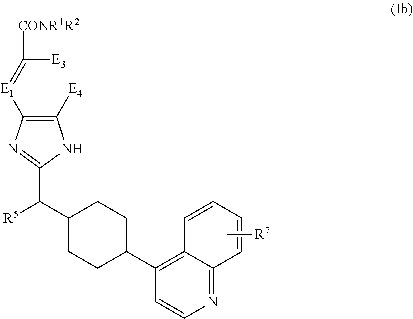
wherein R⁵ is C₁₋₄alkyl, C₁₋₄haloalkyl, C₁₋₄alkoxy, or C₃₋₆cycloalkyl; R⁷ is halogen, R¹, R², E₁, E₃ and E₄ are defined as for Formula (I).

12. The compound of any one of claim 1 or 2 or 9, wherein is selected from one of the following configurations: 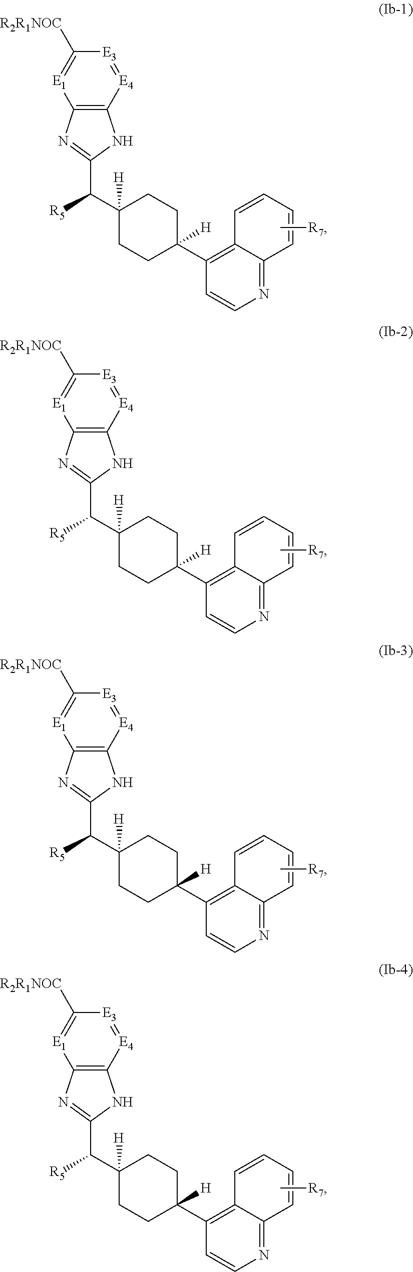
wherein R⁵ is C₁₋₄alkyl, C₁₋₄haloalkyl, C₁₋₄alkoxy, or C₃₋₆cycloalkyl; R⁷ is halogen. R¹, R², E₁, E₃ and E₄ are defined as for Formula (I).

13. The compound of any one of claim 1 or 2 or 9-12, wherein R¹ and R² are each independently H, C₁₋₈ alkyl, C₃₋₈cycloalkyl, aryl, heterocyclyl or heteroaryl, wherein said C₁₋₈ alkyl, C₃₋₈ cycloalkyl, or aryl are each independently optionally substituted with 1 or 2 substituents R¹⁰, or R¹ and R², together with the nitrogen atom to which they are attached, form a 3-, 4-, 5-, or 6-membered saturated ring comprising 0 additional heteroatom, and said ring is optionally substituted with at least one substituent R¹⁰.

14. The compound of claim 13, wherein R¹ is hydrogen or methyl, R² is independently selected from methyl, ethyl, n-propyl, isopropyl, n-butyl, isobutyl, s-butyl, t-butyl, methoxy, cyclopropyl, cyclobutyl, cyclopentyl, cyclohexyl, phenyl, methoxyethyl, hydroxycyclobutylmethyl, oxetanyl.

15. The compound of any one of claims 1-2 or 11-14, wherein ring A is phenyl or naphthalenyl ring; or a monocyclic or bicyclic aromatic heterocyclic ring having 5- to 10-ring members comprising 1, 2, 3, or 4 heteroatoms selected from O, S, and N.

16. The compound of claim 15, wherein ring A is a monocyclic aromatic heterocyclic ring having 5- to 6-ring members comprising 1 or 2 heteroatoms selected from O, S, and N.

17. The compound of claim 15, wherein ring A is a bicyclic aromatic heterocyclic ring having 8- to 10-ring members comprising 1 or 2 or 3 heteroatoms selected from O, S, and N.

18. The compound of claim 17, wherein ring A is benzothiophenyl (such as benzo[b]thiophen-2-yl, benzo[b]thiophen-3-yl, benzo[b]thiophen-5-yl, or benzo[b]thiophen-6-yl) or quinolinyl (such as quinolin-2-yl, quinolin-3-yl, quinolin-4-yl, quinolin-5-yl, quinolin-6-yl, quinolin-7-yl, quinolin-8-yl) or benzodioxolyl (such as benzo[d][1,3]dioxol-5-yl).

19. The compound of claim 18, wherein ring A is quinolinyl (such as quinolin-2-yl, quinolin-3-yl, quinolin-4-yl, quinolin-5-yl, quinolin-6-yl, quinolin-7-yl, quinolin-8-yl) optionally substituted with halogen or C₁₋₈haloalkyl.

20. The compound of claim 1, wherein the compound is selected from the following group consisting of:

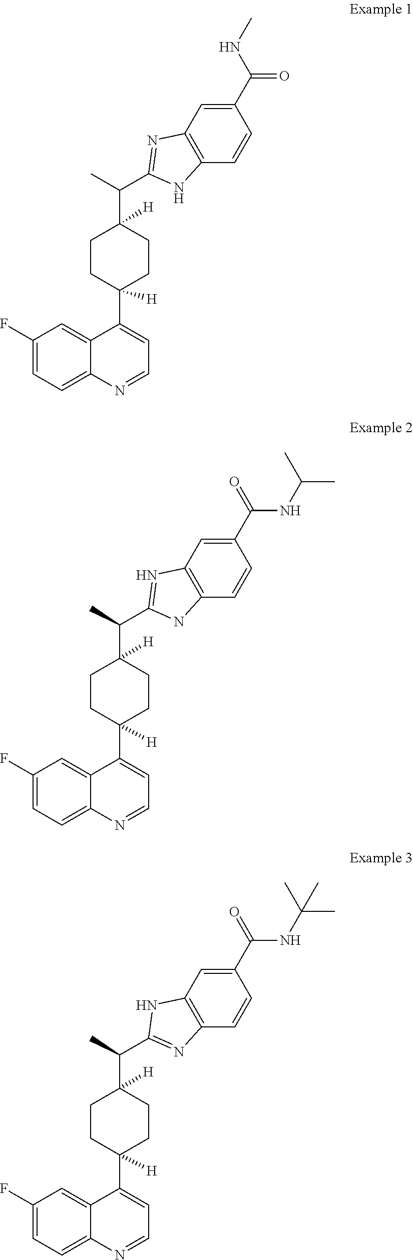 [SMILES: CNC(=O)c1ccc2[nH]c(C(C)[C@H]3CC[C@@H](c4ccnc5ccc(F)cc54)CC3)nc2c1 ; CC(C)NC(=O)c1ccc2[nH]c([C@H](C)[C@H]3CC[C@@H](c4ccnc5ccc(F)cc54)CC3)nc2c1 ; C[C@@H](c1nc2ccc(C(=O)NC(C)(C)C)cc2[nH]1)[C@H]1CC[C@@H](c2ccnc3ccc(F)cc32)CC1]

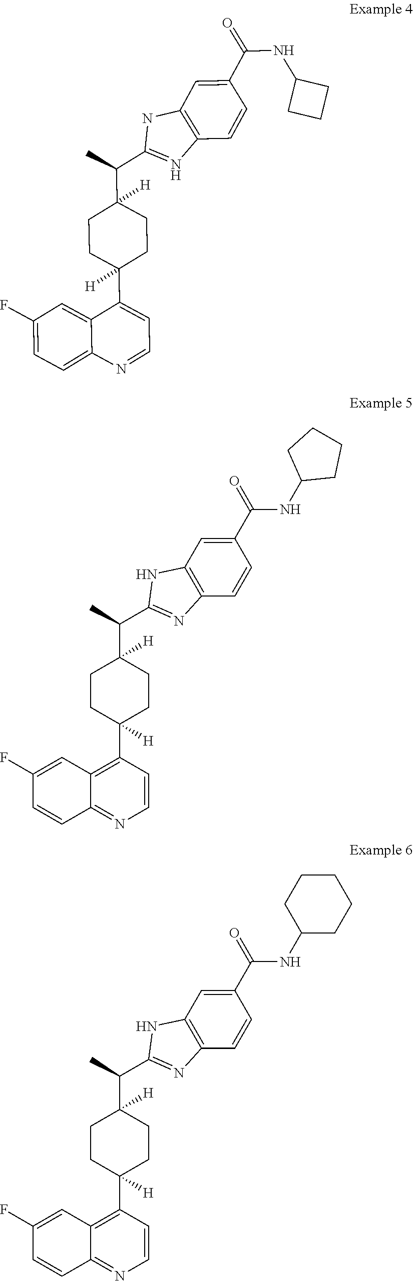 [SMILES: C[C@@H](c1nc2ccc(C(=O)NC3CCC3)cc2[nH]1)[C@H]1CC[C@@H](c2ccnc3ccc(F)cc32)CC1 ; C[C@@H](c1nc2ccc(C(=O)NC3CCCC3)cc2[nH]1)[C@H]1CC[C@@H](c2ccnc3ccc(F)cc32)CC1 ; C[C@@H](c1nc2ccc(C(=O)NC3CCCCC3)cc2[nH]1)[C@H]1CC[C@@H](c2ccnc3ccc(F)cc32)CC1]

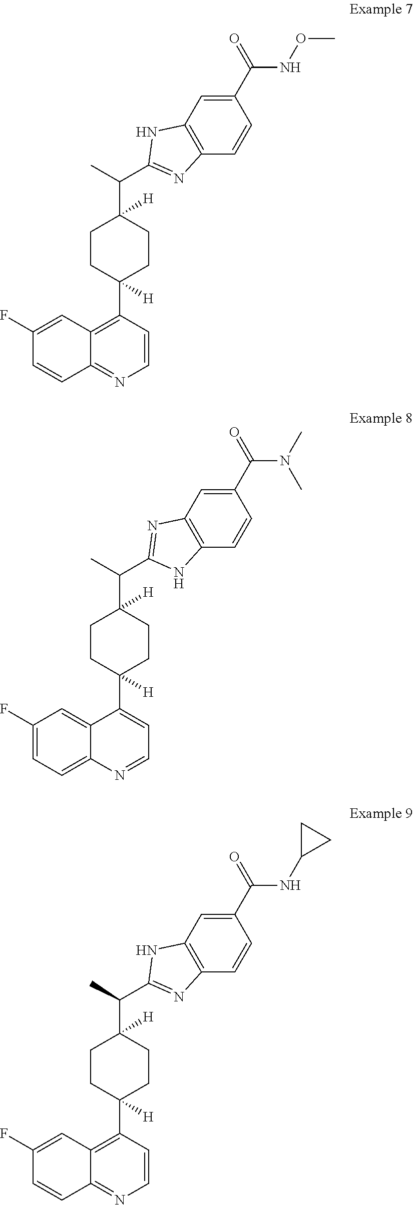 [SMILES: CONC(=O)c1ccc2nc(C(C)[C@H]3CC[C@@H](c4ccnc5ccc(F)cc54)CC3)[nH]c2c1 ; CC(c1nc2cc(C(=O)N(C)C)ccc2[nH]1)[C@H]1CC[C@@H](c2ccnc3ccc(F)cc32)CC1 ; C[C@@H](c1nc2ccc(C(=O)NC3CC3)cc2[nH]1)[C@H]1CC[C@@H](c2ccnc3ccc(F)cc32)CC1]

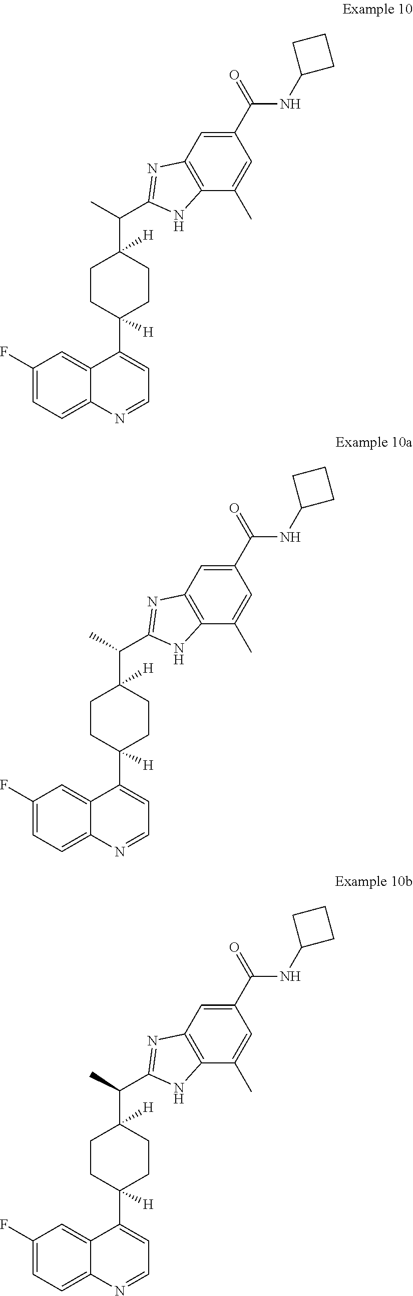 [SMILES: Cc1cc(C(=O)NC2CCC2)cc2nc(C(C)[C@H]3CC[C@@H](c4ccnc5ccc(F)cc54)CC3)[nH]c12 ; Cc1cc(C(=O)NC2CCC2)cc2nc([C@@H](C)[C@H]3CC[C@@H](c4ccnc5ccc(F)cc54)CC3)[nH]c12 ; Cc1cc(C(=O)NC2CCC2)cc2nc([C@H](C)[C@H]3CC[C@@H](c4ccnc5ccc(F)cc54)CC3)[nH]c12]

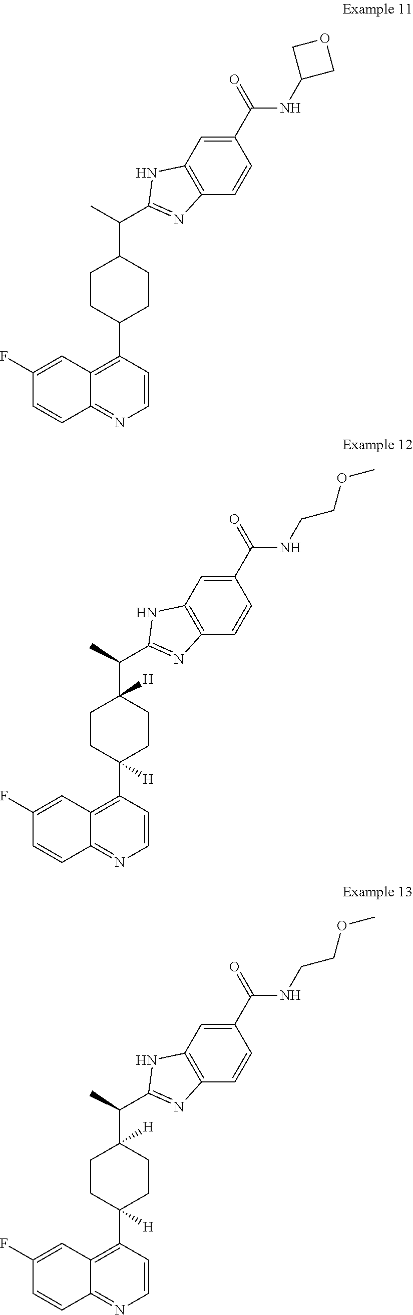 [SMILES: CC(c1nc2ccc(C(=O)NC3COC3)cc2[nH]1)C1CCC(c2ccnc3ccc(F)cc23)CC1 ; COCCNC(=O)c1ccc2nc([C@H](C)[C@H]3CC[C@H](c4ccnc5ccc(F)cc54)CC3)[nH]c2c1 ; COCCNC(=O)c1ccc2nc([C@H](C)[C@H]3CC[C@@H](c4ccnc5ccc(F)cc54)CC3)[nH]c2c1]

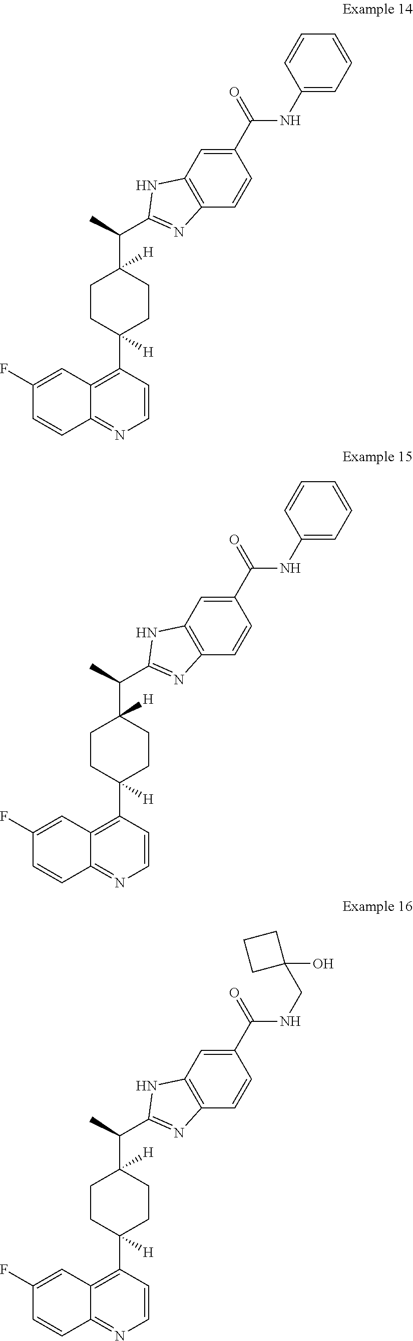 [SMILES: C[C@@H](c1nc2ccc(C(=O)Nc3ccccc3)cc2[nH]1)[C@H]1CC[C@@H](c2ccnc3ccc(F)cc32)CC1 ; C[C@@H](c1nc2ccc(C(=O)Nc3ccccc3)cc2[nH]1)[C@H]1CC[C@H](c2ccnc3ccc(F)cc32)CC1 ; C[C@@H](c1nc2ccc(C(=O)NCC3(O)CCC3)cc2[nH]1)[C@H]1CC[C@@H](c2ccnc3ccc(F)cc32)CC1]

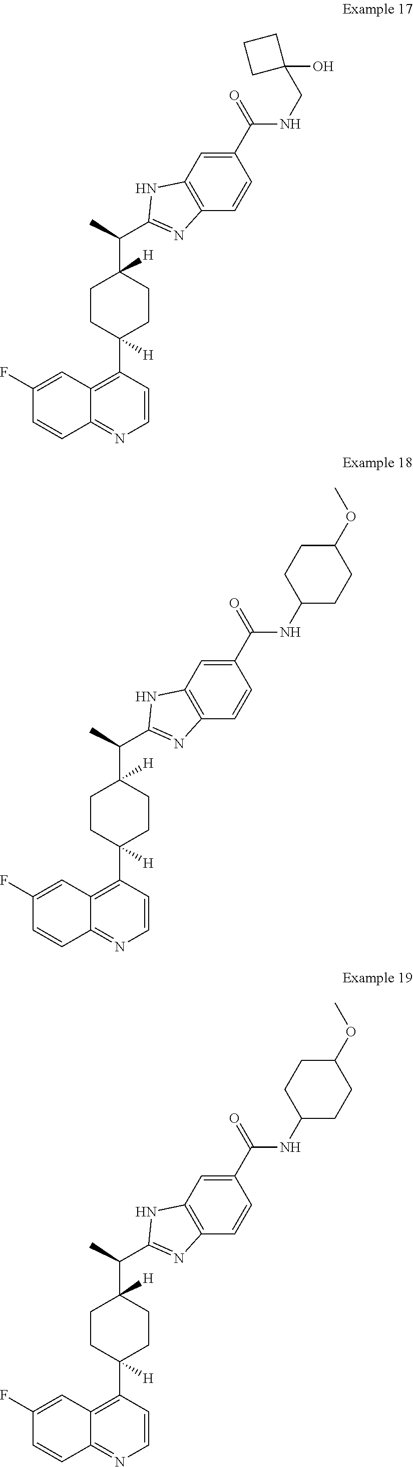 [SMILES: C[C@@H](c1nc2ccc(C(=O)NCC3(O)CCC3)cc2[nH]1)[C@H]1CC[C@H](c2ccnc3ccc(F)cc32)CC1 ; COC1CCC(NC(=O)c2ccc3nc([C@H](C)[C@H]4CC[C@@H](c5ccnc6ccc(F)cc65)CC4)[nH]c3c2)CC1 ; COC1CCC(NC(=O)c2ccc3nc([C@H](C)[C@H]4CC[C@H](c5ccnc6ccc(F)cc65)CC4)[nH]c3c2)CC1]

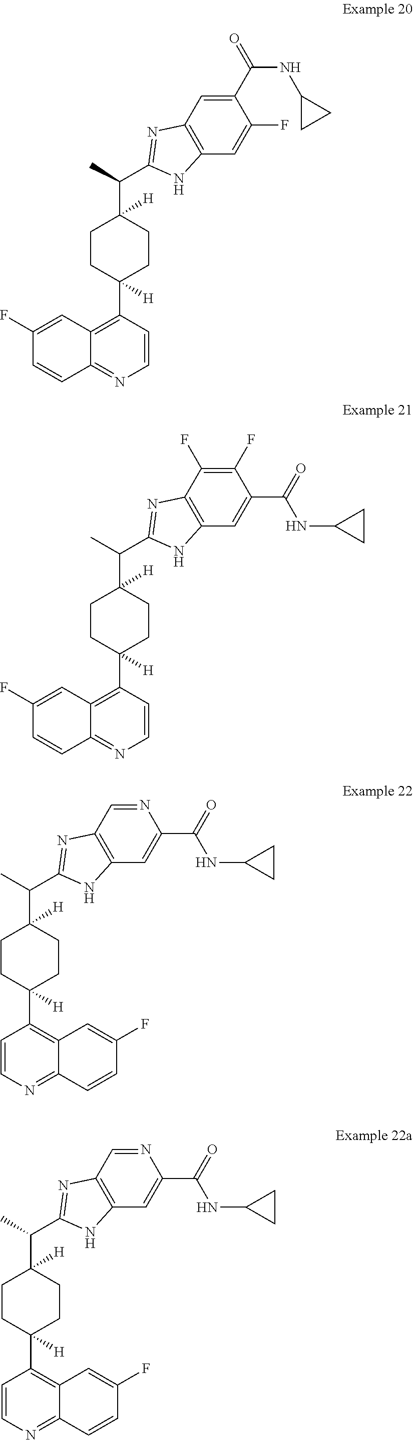 [SMILES: C[C@@H](c1nc2cc(C(=O)NC3CC3)c(F)cc2[nH]1)[C@H]1CC[C@@H](c2ccnc3ccc(F)cc32)CC1 ; CC(c1nc2c(F)c(F)c(C(=O)NC3CC3)cc2[nH]1)[C@H]1CC[C@@H](c2ccnc3ccc(F)cc32)CC1 ; CC(c1nc2cnc(C(=O)NC3CC3)cc2[nH]1)[C@H]1CC[C@@H](c2ccnc3ccc(F)cc32)CC1 ; C[C@H](c1nc2cnc(C(=O)NC3CC3)cc2[nH]1)[C@H]1CC[C@@H](c2ccnc3ccc(F)cc32)CC1]

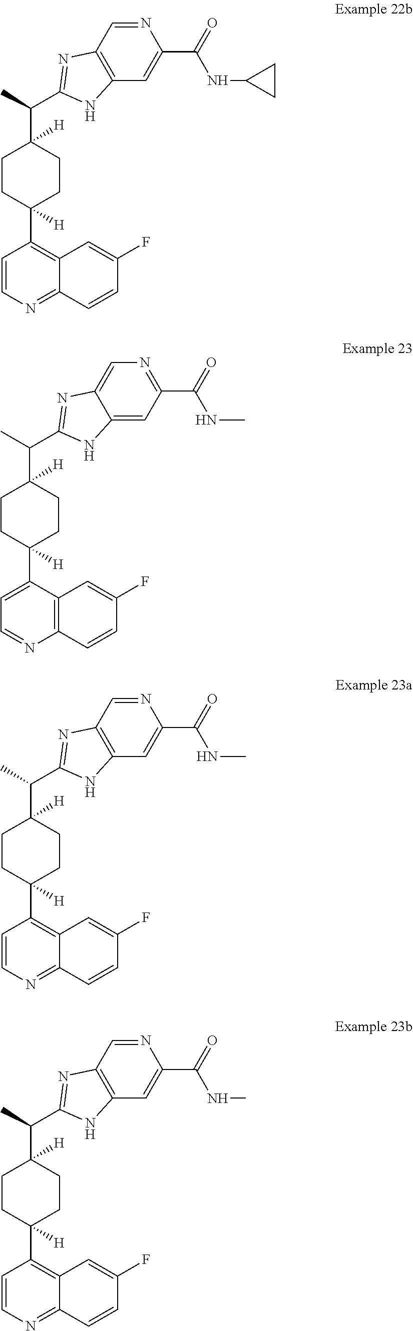 [SMILES: C[C@@H](c1nc2cnc(C(=O)NC3CC3)cc2[nH]1)[C@H]1CC[C@@H](c2ccnc3ccc(F)cc32)CC1 ; CNC(=O)c1cc2[nH]c(C(C)[C@H]3CC[C@@H](c4ccnc5ccc(F)cc54)CC3)nc2cn1 ; CNC(=O)c1cc2[nH]c([C@@H](C)[C@H]3CC[C@@H](c4ccnc5ccc(F)cc54)CC3)nc2cn1 ; CNC(=O)c1cc2[nH]c([C@H](C)[C@H]3CC[C@@H](c4ccnc5ccc(F)cc54)CC3)nc2cn1]

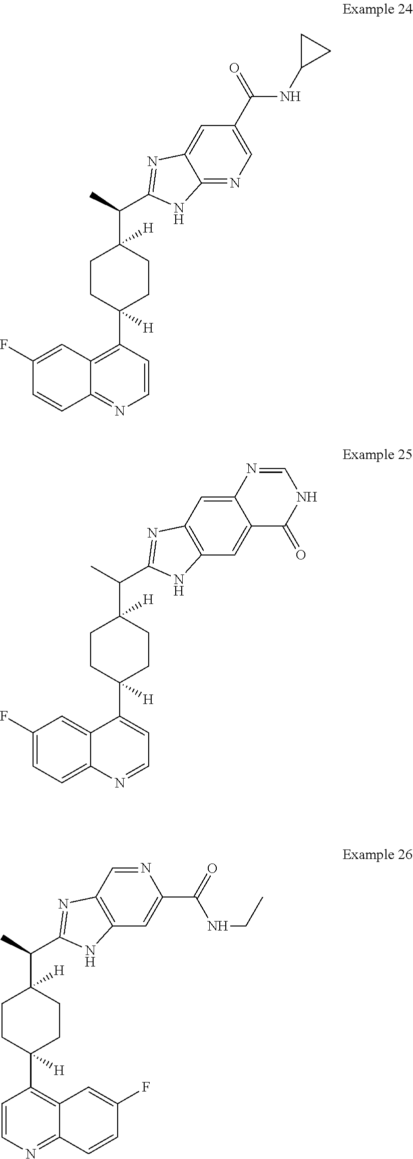 [SMILES: C[C@@H](c1nc2cc(C(=O)NC3CC3)cnc2[nH]1)[C@H]1CC[C@@H](c2ccnc3ccc(F)cc32)CC1 ; CC(c1nc2cc3nc[nH]c(=O)c3cc2[nH]1)[C@H]1CC[C@@H](c2ccnc3ccc(F)cc32)CC1 ; CCNC(=O)c1cc2[nH]c([C@H](C)[C@H]3CC[C@@H](c4ccnc5ccc(F)cc54)CC3)nc2cn1]

21. A pharmaceutical composition comprising at least one pharmaceutically acceptable excipient and a therapeutically effective amount of a compound of any one of claims 1-20, or a stereoisomer thereof, or a pharmaceutically acceptable salt thereof.

22. A method for treating or preventing hyperproliferative disorders responsive to inhibition of IDO1 comprising administering to a subject in recognized need thereof a compound of any one of claims 1-19 or a stereoisomer thereof, or a pharmaceutically acceptable salt thereof in an amount effective to inhibit said IDO1.

23. The method according to claim 22, wherein the hyperproliferative disorder is cancer.

24. The method according to claim 23, wherein the hyperproliferative disorder is selected from melanomas, thyroid cancer, Barret's adenocarcinoma, breast cancer, cervical cancer, colorectal cancer, gastric cancer, lung cancer, renal carcinoma, head and neck cancer, liver cancer, stomach cancer, esophageal cancer, ovarian cancer, pancreatic cancer, prostate cancer, hematologic cancers, cancer of Biliary Tract, Non-small cell lung cancer, endometrium cancer, blood cancer, large intestinal colon carcinoma, histiocytic lymphoma, or lung adenocarcinoma.

25. A method for treating or preventing HIV infection/AIDS comprising administering to a subject in recognized need thereof therapeutically effective amount of a compound of any one of claims 1-20, or a stereoisomer thereof, or a pharmaceutically acceptable salt thereof.

26. A method for enhancing the effectiveness of an anti-retroviral therapy comprising administering to a subject in recognized need thereof therapeutically effective amount of a compound of any one of claims 1-20, or a stereoisomer thereof, or a pharmaceutically acceptable salt thereof.
